(19) United States
(12) Patent Application Publication
(10) Pub. No.: US 2004/0018592 A1
(43) Pub. Date: Jan. 29, 2004

(54) Bovine collagens and gelatins

(75) Inventors: Marcum P. Bell, Nashville, TN (US); Thomas B. Neff, Atherton, CA (US); James W. Polarek, Sausalito, CA (US); Todd W. Seeley, Moraga, CA (US)

Correspondence Address: Leanne C. Price, Esq., FibroGen, Inc., 225 Gateway Blvd., South San Francisco, CA, 94080, US

(73) Assignee: FibroGen, Inc.

(21) Appl. No.: 10/402,072

(22) Filed: Mar. 26, 2003

Related U.S. Application Data
(63) Continuation of application No. 09/709,700, filed on Nov. 10, 2000.
Application No. 09/709,700 is a continuation-in-part of application No. 09/439,058, filed on Nov. 12, 1999, now abandoned.

(51) Int. Cl. C12P 21/02; C12N 5/06; A61K 38/17; C07K 14/78

(52) U.S. Cl. 435/69.1; 514/12; 435/320.1; 435/325; 530/356

(57) ABSTRACT

The present invention provides animal collagens and gelatins and compositions thereof, and methods of producing the same.

This application is a continuation of U.S. application Ser. No. 09/709,700, filed Nov. 10, 2000, which is a continuation-in-part application of U.S. application Ser. No. 09/439,058, filed Nov. 12, 1999, each of which is incorporated by reference herein in its entirety.

### FIELD OF THE INVENTION

The present invention relates to the recombinant synthesis of collagens and gelatins derived from animal sequences. The present invention also relates to novel polynucleotide sequences encoding bovine and porcine collagens, and to the encoded polypeptide sequences, and to the use of such sequences in the recombinant production of animal collagens and gelatins.

### BACKGROUND OF THE INVENTION

The most abundant component of the extracellular matrix is collagen. Collagens are a large family of fibrous proteins, characterized by the presence of triple-stranded helical domains. Collagen molecules are generally the result of the trimeric assembly of polypeptide chains containing (-Gly-X—Y—)ₙ repeats which allow for the formation of triple helical domains (van der Rest et al. (1991) FASEB J. 5:2814-2823).

### Collagen

Presently, about twenty distinct collagen types have been identified in vertebrates, including bovine, ovine, porcine, chicken, and human collagens. Generally, the collagen types are numbered by Roman numerals, and the chains found in each collagen type are identified by Arabic numerals. Detailed descriptions of structure and biological functions of the various different types of naturally occurring collagens are generally available in the art. (See, e.g., Ayad et al. (1998) The Extracellular Matrix Facts Book, Academic Press, San Diego, Calif.; Burgeson, R. E., and Nimmi (1992) “Collagen types: Molecular Structure and Tissue Distribution” in Clin. Orthop. 282:250-272; Kielty, C. M. et al. (1993) “The Collagen Family: Structure, Assembly And Organization In The Extracellular Matrix,” Connective Tissue And Its Heritable Disorders, Molecular Genetics, And Medical Aspects, Royce, P. M. and B. Steinmann eds., Wiley-Liss, NY, pp. 103-147; and Prockop, D. J. and K. I. Kivirikko (1995) “Collagens: Molecular Biology, Diseases, and Potentials for Therapy,” Annu. Rev. Biochem., 64:403-434.)

Type I collagen is the major fibrillar collagen of bone and skin, comprising approximately 80-90% of an organism's total collagen. Type I collagen is the major structural macromolecule present in the extracellular matrix of multicellular organisms and comprises approximately 20% of total protein mass. Type I collagen is a heterotrimeric molecule comprising two α1(I) chains and one α2(I) chain, encoded by the COL1A1 and COL1A2 genes, respectively. Other collagen types are less abundant than type I collagen, and exhibit different distribution patterns. For example, type II collagen is the predominant collagen in cartilage and vitreous humor, while type III collagen is found at high levels in blood vessels and to a lesser extent in skin.

Type II collagen is a homotrimeric collagen comprising three identical α1(II) chains encoded by the COL2A1 gene. Purified type II collagen may be prepared from tissues by, methods known in the art, for example, by procedures described in Miller and Rhodes (1982) Methods In Enzymology 82:33-64.

Type III collagen is a major fibrillar collagen found in skin and vascular tissues. Type III collagen is a homotrimeric collagen comprising three identical α1(III) chains encoded by the COL3A1 gene. Methods for purifying type III collagen from tissues can be found in, for example, Byers et al. (1974) Biochemistry 13:5243-5248; and Miller and Rhodes, supra.

Type IV collagen is found in basement membranes in the form of sheets rather than fibrils. Most commonly, type IV collagen contains two α1(IV) chains and one α2(IV) chain. The particular chains comprising type IV collagen are tissue-specific. Type IV collagen may be purified using, for example, the procedures described in Furuto and Miller (1987) Methods in Enzymology, 144:41-61, Academic Press.

Type V collagen is a fibrillar collagen found in, primarily, bones, tendon, cornea, skin, and blood vessels. Type V collagen exists in both homotrimeric and heterotrimeric forms. One form of type V collagen is a heterotrimer of two α1(V) chains and one α2(V) chain. Another form of type V collagen is a heterotrimer of α1(V), α2(V), and α3(V) chains. A further form of type V collagen is a homotrimer of α1(V). Methods for isolating type V collagen from natural sources can be found, for example, in Elstow and Weiss (1983) Collagen Rel. Res. 3:181-193, and Abedin et al. (1982) Biosci. Rep. 2:493-502.

Type VI collagen has a small triple helical region and two large non-collagenous remainder portions. Type VI collagen is a heterotrimer comprising α1(VI), α2(VI), and α3(VI) chains. Type VI collagen is found in many connective tissues. Descriptions of how to purify type VI collagen from natural sources can be found, for example, in Wu et al. (1987) Biochem. J. 248:373-381, and Kielty et al. (1991) J. Cell Sci. 99:797-807.

Type VII collagen is a fibrillar collagen found in particular epithelial tissues. Type VII collagen is a homotrimeric molecule of three α1(VII) chains. Descriptions of how to purify type VII collagen from tissue can be found in, for example, Lunstrum et al. (1986) J. Biol. Chem. 261:9042-9048, and Bentz et al. (1983) Proc. Natl. Acad. Sci. USA 80:3168-3172.

Type VIII collagen can be found in Descemet's membrane in the cornea. Type VIII collagen is a heterotrimer comprising two α1(VIII) chains and one α2(VIII) chain, although other chain compositions have been reported. Methods for the purification of type VIII collagen from nature can be found, for example, in Benya and Padilla (1986) J. Biol. Chem. 261:4160-4169, and Kapoor et al. (1986) Biochemistry 25:3930-3937.

Type IX collagen is a fibril-associated collagen found in cartilage and vitreous humor. Type IX collagen is a heterotrimeric molecule comprising α1(IX), α2(IX), and α3(IX) chains. Type IX collagen has been classified as a FACIT (Fibril Associated Collagens with Interrupted Triple Helices) collagen, possessing several triple helical domains separated by non-triple helical domains. Procedures for purifying type IX collagen can be found, for example, in Duance, et al. (1984) Biochem. J. 221:885-889; Ayad et al. (1989) Biochem. J. 262:753-761; and Grant et al. (1988) The Control of Tissue Damage, Glauert, A. M., ed., Elsevier Science Publishers, Amsterdam, pp. 3-28.

Type X collagen is a homotrimeric compound of α1(X) chains. Type X collagen has been isolated from, for example, hypertrophic cartilage found in growth plates. (See, e.g., Apte et al. (1992) Eur J Biochem 206 (1):217-24.)

Type XI collagen can be found in cartilaginous tissues associated with type II and type IX collagens, and in other locations in the body. Type XI collagen is a heterotrimeric molecule comprising α1(XI), α2(XI), and α3(XI) chains. Methods for purifying type XI collagen can be found, for example, in Grant et al., supra.

Type XII collagen is a FACIT collagen found primarily in association with type I collagen. Type XII collagen is a homotrimeric molecule comprising three α1(XII) chains. Methods for purifying type XII collagen and variants thereof can be found, for example, in Dublet et al. (1989) J. Biol. Chem. 264:13150-13156; Lunstrum et al. (1992) J. Biol. Chem. 267:20087-20092; and Watt et al. (1992) J. Biol. Chem. 267:20093-20099.

Type XIII is a non-fibrillar collagen found, for example, in skin, intestine, bone, cartilage, and striated muscle. A detailed description of type XIII collagen may be found, for example, in Juvonen et al. (1992) J. Biol. Chem. 267:24700-24707.

Type XIV is a FACIT collagen characterized as a homotrimeric molecule comprising α1(XIV) chains. Methods for isolating type XIV collagen can be found, for example, in Aubert-Foucher et al. (1992) J. Biol. Chem. 267:15759-15764, and Watt et al., supra.

Type XV collagen is homologous in structure to type XVIII collagen. Information about the structure and isolation of natural type XV collagen can be found, for example, in Myers et al. (1992) Proc. Natl. Acad. Sci. USA 89:10144-10148; Huebner et al. (1992) Genomics 14:220-224; Kivirikko et al. (1994) J. Biol. Chem. 269:4773-4779; and Muragaki, J. (1994) Biol. Chem. 264:4042-4046.

Type XVI collagen is a fibril-associated collagen, found, for example, in skin, lung fibroblast, and keratinocytes. Information on the structure of type XVI collagen and the gene encoding type XVI collagen can be found, for example, in Pan et al. (1992) Proc. Natl. Acad. Sci. USA 89:6565-6569; and Yamaguchi et al. (1992) J. Biochem. 112:856-863.

Type XVII collagen is a hemidesmosal transmembrane collagen, also known at the bullous pemphigoid antigen. Information on the structure of type XVII collagen and the gene encoding type XVII collagen can be found, for example, in Li et al. (1993) J. Biol. Chem. 268(12):8825-8834; and McGrath et al. (1995) Nat. Genet. 1 1(1):83-86.

Type XVIII collagen is similar in structure to type XV collagen and can be isolated from the liver. Descriptions of the structures and isolation of type XVIII collagen from natural sources can be found, for example, in Rehn and Pihlajaniemi (1994) Proc. Natl. Acad. Sci USA 91:4234-4238; Oh et al. (1994) Proc. Natl. Acad. Sci USA 91:4229-4233; Rehn et al. (1994) J. Biol. Chem. 269:13924-13935; and Oh et al. (1994) Genomics 19:494-499.

Type XIX collagen is believed to be another member of the FACIT collagen family, and has been found in mRNA isolated from rhabdomyosarcoma cells. Descriptions of the structures and isolation of type XIX collagen can be found, for example, in Inoguchi et al. (1995) J. Biochem. 117:137-146; Yoshioka et al. (1992) Genomics 13:884-886; and Myers et al., J. Biol. Chem. 289:18549-18557 (1994).

Type XX collagen is a newly found member of the FACIT collagenous family, and has been identified in chick cornea. (See, e.g., Gordon et al. (1999) FASEB Journal 13A1119; and Gordon et al. (1998), IOVS 39:S1128.)

### Gelatin

Gelatin is a derivative of collagen, a principal structural and connective protein in animals. Gelatin is derived from denaturation of collagen and contains polypeptide sequences having Gly-X—Y repeats, where X and Y are most often proline and hydroxyproline residues. These sequences contribute to triple helical structure and affect the gelling ability of gelatin polypeptides. Currently available gelatin is extracted through processing of animal hides and bones, typically from bovine and porcine sources. The biophysical properties of gelatin make it a versatile material, widely used in a variety of applications and industries. Gelatin is used, for example, in numerous pharmaceutical and medical, photographic, industrial, cosmetic, and food and beverage products and processes of manufacture. Gelatin is thus a commercially valuable and versatile product.

Gelatin is typically manufactured from naturally occurring collagen in bovine and porcine sources, in particular, from hides and bones. In some instances, gelatin can be extracted from, for example, piscine, chicken, or equine sources. Raw materials of typical gelatin production, such as bovine hides and bones, originate from animals subject to government-certified inspection and passed as fit for human consumption. There is concern over the infectivity of this raw material, due to the presence of contaminating agents such as transmissible spongifoim encephalopathies (TSEs), particularly bovine spongiform encephalopathy (BSE), and scrapie, etc. (See, e.g., Rohwer, R. G. (1996), Dev Biol Stand 88:247-256.) Such issues are especially critical to gelatin used in pharmaceutical and medical applications.

Recently, concern about the safety of these materials, a significant portion of which are derived from bovine sources, has increased, causing various gelatin-containing products to become the focus of several regulatory measures to reduce the potential risk of transmission of bovine spongiform encephalopathy (BSE), linked to new variant Creutzfeldt-Jakob disease (nvCJD), a fatal neurological disease in humans. There is concern that purification steps currently used in the process of extracting gelatin from animal tissues and bones may not be sufficient to remove the likelihood of infectivity due to contaminating SE-carrying tissue (i.e., brain tissue, etc.). U.S. and European manufacturers specify that raw material for gelatin to be included in animal or human food products or in pharmaceutical, medical, or cosmetic applications must not be obtained from a growing number of BSE countries. In addition, regulations specify that certain materials, e.g., bovine brain tissues, are not used in the production of gelatin.

Current production processes involve several purification and cleansing steps, and can require harsh and lengthy modes of extraction. The animal hides and bones are treated in a rendering process, and the extracted material is subjected to various chemical treatments, including prolonged exposure to highly acidic or alkaline solutions. Numerous purification steps can involve washing and filtration and various heat treatments. Acid demineralization and lime treatments are used to remove impurities such as non-collagenous proteins Bones must be degreased. Additional washing and filtration steps, ion exchanges, and other chemical and sterilizing treatments are added to the process to further purify the material. Furthermore, contaminants and impurities can still remain after processing, and the resultant gelatin product must thus typically be clarified, purified, and often further concentrated before being ready for use.

Commercial gelatin is generally classified as type A or type B. These classifications reflect the pre-treatment extraction sources receive as part of the extraction process. Type A is generally derived from acid-processed materials, usually porcine hides, and type B is generally derived from alkaline- or lime-processed materials, usually bovine bones (ossein) and hides. In both type A and B extraction processes, the resultant gelatin product typically comprises a mixture of gelatin molecules, in sizes of from a few thousand up to several hundred thousand Daltons.

Fish gelatin, classified as gelling or non-gelling types, and typically processed as Type A gelatin, is also used in certain commercial applications. Gelling types are usually derived from the skins of warm water fish, while non-gelling types are typically derived from cold water fish. Fish gelatins have widely varying amino acid compositions, and differ from animal gelatins in having typically lower proportions of proline and hydroxyproline residues. In contrast to other animal gelatins, fish gelatins typically remain liquid at much lower temperatures, even at comparable average molecular weights. As with animal gelatin, fish gelatin is extracted by treatment and subsequent hydrolyzation of fish skin. Again, as with animal extraction processes, the process of extracting fish gelatin results in a product that lacks homogeneity.

Current methods of extraction thus result in a gelatin product that is a heterogeneous mixture of proteins, containing polypeptides with molecular weight distributions of varying ranges. It is sometimes necessary to blend various lots of product in order to obtain a gelatin mixture with the physical properties appropriate for use in a desired application. There is thus a need for a reliable and reproducible means of gelatin production that provides a homogenous product with controlled characteristics.

In addition, in the pharmaceutical, cosmetic, and food and beverage industries, especially, there is a need for a source of gelatin other than that obtained through extraction from animal sources, e.g., bovine, porcine bones and tissues. Further, as currently available gelatin is manufactured from animal sources such as bones and tissues, there are concerns relating to the undesirable immunogenicity and infectivity of gelatin-containing products. (See, e.g., Sakaguchi, M. et al. (1999) J. Aller. Clin. Immunol. 104:695-699; Miyazawa et al. (1999) Vaccine 17:2176-2180; Sakaguchi et al. (1999) Immunology 96:286-290; Kelso (1999) J Aller. Clin Immunol. 103:200-202; Asher (1999) Dev Biol Stand 99:41-44; and Verdrager (1999) Lancet 354:1304-1305.) In addition, the availability of a substitute material that does not undergo extraction from animal sources, e.g., tissues and bones, will address various ethical, religious, and social dictates. A recombinant material that does not require extraction from animal sources, such as tissues and bones, could be used, for example, in the manufacture of foods and other ingested products, including encapsulated medicines, that are appropriate for use by people with dietary restrictions, for example, those who follow Kosher and Halal law.

### Post-translational Enzymes

Post-translational enzymes are important to the biosynthesis of collagens and collagenous proteins. For example, prolyl 4-hydroxylase is required to hydroxylate prolyl residues in the Y-position of the repeating -Gly-X—Y— sequences to 4-hydroxyproline. (See, e.g., Prockop et al. (1984) N. Engl. J. Med. 311:376-386.) Hydroxyproline plays a critical role for stabilization of the collagen triple helix.

Vertebrate prolyl 4-hydroxylase is an α₂β₂ tetramer. (See, e.g. Berg and Prockop. (1973) J. Biol. Chem. 248:1175-1192; and Tuderman et al. (1975) Eur. J. Biochem. 52:9-16.) The α subunits (63 kDa) contain the catalytic sites involved in the hydroxylation of prolyl residues, and are insoluble in the absence of β subunits. The β subunits (55 kDa), identical to protein disulfide isomerase, catalyze thiol/disulfide interchange protein substrate, leading to the formation of a set of disulfide bonds essential to establishing a stable protein. The β subunits retain 50% of protein disulfide isomerase activity when part of the prolyl 4-hydroxylase tetramer. (See, e.g., Pihlajaniemi et al. (1987) Embo J. 6:643-649; Parkkonen et al. (1988) Biochem. J. 256:1005-1011; and Koivu et al. (1987) J. Biol. Chem. 262:6447-6449.) Active recombinant human prolyl 4-hydroxylase has been produced in insect cells by simultaneously expressing the α and β subunits. (See, e.g., Vuori et al. (1992) Proc. Natl. Acad. Sci. USA 89:7467-7470.)

In addition to prolyl 4-hydroxylase, other collagen post-translational enzymes have been identified and reported in the literature, including, for example, C-proteinase, N-proteinase, lysyl oxidase, and lysyl hydroxylase. (See, e.g., Olsen et al. (1991) Cell Biology of Extracellular Matrix, 2^{nd} ed., Hay editor, Plenum Press, New York.)

Expression of many exogenous genes is readily obtained in a variety of recombinant host-vector systems. However, expression becomes difficult if the final formation of the protein requires extensive post-translational processing. For example, prolyl 4-hydroxylase activity is clearly an essential requirement for hydroxylation in nature of collagenous domains. Supplementation of prolyl 4-hydroxylase activity is required in expression systems deficient of prolyl 4-hydroxylase endogenous activity, in order to provide hydroxylation systems as found in nature.

Failure to obtain reliable and stable recombinant expression of genes for collagens has prevented the production of collagens and gelatins that have a number of useful applications. In addition, many types of collagen are only available in trace quantities present in tissues, and cannot be obtained in significant quantities from these sources. Furthermore, non-collagenous impurities can be left over after or introduced during the extraction and purification processes.

### SUMMARY

In summary, although the characteristics of commercially available animal collagens and gelatins are suitable for many products, the variability in these currently available materials, and the difficulties associated with optimizing these materials for use in various applications, provide little flexibility. As a result, there is a need in the art for an efficient system that allows the starting material to be modified at the genetic and molecular levels, providing the potential for producing recombinant collagens and gelatins, specifically tailored and standardized for different applications and markets. Furthermore, existing concern over the risks of immunogenicity and infectivity associated with the use of the extracted materials currently available has established a need for a pure and safe substitute material.

### SUMMARY OF THE INVENTION

The present invention provides animal collagens and gelatins, and methods of producing these animal collagens and gelatins. Therefore, in one aspect, the present invention encompasses an isolated and purified polypeptide comprising a bovine or porcine polypeptide selected from the group consisting of α1(I) collagens, α2(I) collagens, and α1(III) collagens, and fragments and variants of these collagens.

In one embodiment, the invention provides an isolated and purified polypeptide comprising a bovine α1(I) collagen or fragments or variants thereof. In certain embodiments, the polypeptide is single-chain, or homotrimeric, or heterotrimeric. In one aspect, the polypeptide comprises the amino acid sequence of SEQ ID NO: 2 or fragments or variants thereof. A composition comprising the polypeptide is also provided.

In a further embodiment, the present invention encompasses an isolated and purified polynucleotide encoding a bovine α1(I) collagen or fragments or variants thereof, and an isolated and purified polynucleotide that is complementary to the polynucleotide encoding a bovine α1(I) collagen or fragments or variants thereof. The present invention provides, in one embodiment, an isolated and purified polynucleotide encoding SEQ ID NO: 2 or fragments or variants thereof. Compositions, expression vectors, and host cells comprising the polynucleotide are also provided. In various embodiments, the host cell is a prokaryotic cell or a eukaryotic cell, specifically, an animal, yeast, plant, insect, or fungal cell. In some embodiments, the present invention provides transgenic animals and transgenic plants comprising the polynucleotide. In one aspect, the present invention encompasses a method for producing a bovine α1(I) collagen, the method comprising culturing the host cell comprising the polynucleotide under conditions suitable for expression of the bovine α (I) collagen, and recovering the bovine α1(1) collagen from the host cell culture.

In certain embodiments, the present invention provides recombinant collagens and recombinant gelatins comprising bovine α1(I) collagen or fragments or variants thereof. The invention specifically provides recombinant collagens and gelatins comprising SEQ ID NO: 2 or fragments or variants thereof.

In one embodiment, the invention provides an isolated and purified polypeptide comprising a bovine α1(III) collagen or fragments or variants thereof. In certain embodiments, the polypeptide is single-chain, or homotrimeric, or heterotrimeric. In one aspect, the polypeptide comprises the amino acid sequence of SEQ ID NO: 4 or SEQ ID NO: 6 or fragments or variants thereof. A composition comprising the polypeptide is also provided.

In a further embodiment, the present invention encompasses an isolated and purified polynucleotide encoding a bovine α1(III) collagen or fragments or variants thereof, and an isolated and purified polynucleotide that is complementary to the polynucleotide encoding a bovine α1(III) collagen or fragments or variants thereof. The present invention provides, in one embodiment, an isolated and purified polynucleotide encoding SEQ ID NO: 4 or SEQ ID NO: 6 or fragments or variants thereof. Compositions, expression vectors, and host cells comprising the polynucleotide are also provided. In various embodiments, the host cell is a prokaryotic cell or a eukaryotic cell, specifically, an animal, yeast, plant, insect, or fungal cell. In some embodiments, the present invention provides transgenic animals and transgenic plants comprising the polynucleotide. In one aspect, the present invention encompasses a method for producing a bovine α1(III) collagen, the method comprising culturing the host cell comprising the polynucleotide under conditions suitable for expression of the bovine α1(III) collagen, and recovering the bovine α1(III) collagen from the host cell culture.

In certain embodiments, the present invention provides recombinant collagens and recombinant gelatins comprising bovine α1(III) collagen or fragments or variants thereof. The invention specifically provides recombinant collagens and gelatins comprising SEQ ID NO: 4 or SEQ ID NO: 6 or fragments or variants thereof.

In one embodiment, the invention provides an isolated and purified polypeptide comprising a porcine α1(I) collagen or fragments or variants thereof. In certain embodiments, the polypeptide is single-chain, or homotrimeric, or heterotrimeric. In one aspect, the polypeptide comprises the amino acid sequence of SEQ ID NO: 8 or fragments or variants thereof. A composition comprising the polypeptide is also provided.

In a further embodiment, the present invention encompasses an isolated and purified polynucleotide encoding a porcine α1(I) collagen or fragments or variants thereof, and an isolated and purified polynucleotide that is complementary to the polynucleotide encoding a porcine α1(I) collagen or fragments or variants thereof. The present invention provides, in one embodiment, an isolated and purified polynucleotide encoding SEQ ID NO: 8 or fragments or variants thereof. Compositions, expression vectors, and host cells comprising the polynucleotide are also provided. In various embodiments, the host cell is a prokaryotic cell or a eukaryotic cell, specifically, an animal, yeast, plant, insect, or fungal cell. In some embodiments, the present invention provides transgenic animals and transgenic plants comprising the polynucleotide. In one aspect, the present invention encompasses a method for producing a porcine α1(I) collagen, the method comprising culturing the host cell comprising the polynucleotide under conditions suitable for expression of the porcine α1(I) collagen, and recovering the porcine α1(I) collagen from the host cell culture.

In certain embodiments, the present invention provides recombinant collagens and recombinant gelatins comprising porcine α1(I) collagen or fragments or variants thereof. The invention specifically provides for recombinant collagens and gelatins comprising SEQ ID NO: 8 or fragments or variants thereof.

In one embodiment, the invention provides an isolated and purified polypeptide comprising a porcine α2(I) collagen or fragments or variants thereof In certain embodiments, the polypeptide is single-chain, or homotrimeric, or heterotrimeric. In one aspect, the polypeptide comprises the amino acid sequence of SEQ ID NO: 10 or fragments or variants thereof. A composition comprising the polypeptide is also provided.

In a further embodiment, the present invention encompasses an isolated and purified polynucleotide encoding a porcine α2(I) collagen or fragments or variants thereof, and an isolated and purified polynucleotide that is complementary to the polynucleotide encoding a porcine α2(I) collagen or fragments or variants thereof. The present invention provides, in one embodiment, an isolated and purified polynucleotide encoding SEQ ID NO: 10 or fragments or variants thereof. Compositions, expression vectors, and host cells comprising the polynucleotide are also provided.

In various embodiments, the host cell is a prokaryotic cell or a eukaryotic cell, specifically, an animal, yeast, plant, insect, or fungal cell. In some embodiments, the present invention provides transgenic animals and transgenic plants comprising the polynucleotide. In one aspect, the present invention encompasses a method for producing a porcine α2(I) collagen, the method comprising culturing the host cell comprising the polynucleotide under conditions suitable for expression of the porcine α2(I) collagen, and recovering the porcine α2(I) collagen from the host cell culture.

In certain embodiments, the present invention provides recombinant collagens and recombinant gelatins comprising porcine α2(I) collagen or fragments or variants thereof. The invention specifically provides for recombinant collagens and gelatins comprising SEQ ID NO: 10 fragments or variants thereof.

In one embodiment, the invention provides an isolated and purified polypeptide comprising a porcine α1(III) collagen or fragments or variants thereof. In certain embodiments, the polypeptide is single-chain, or homotrimeric, or heterotrimeric. In one aspect, the polypeptide comprises the amino acid sequence of SEQ ID NO: 12 or fragments or variants thereof. A composition comprising the polypeptide is also provided.

In a further embodiment, the present invention encompasses an isolated and purified polynucleotide encoding a porcine α1(III) collagen or fragments or variants thereof, and an isolated and purified polynucleotide that is complementary to the polynucleotide a porcine α1(III) collagen or fragments or variants thereof. The present invention provides, in one embodiment, an isolated and purified polynucleotide encoding SEQ ID NO: 12 or fragments or variants thereof.

Compositions, expression vectors, and host cells comprising the polynucleotide are also provided. In various embodiments, the host cell is a prokaryotic cell or a eukaryotic cell, specifically, an animal, yeast, plant, insect, or fungal cell. In some embodiments, the present invention provides transgenic animals and transgenic plants comprising the polynucleotide. In one aspect, the present invention encompasses a method for producing a porcine α1(III) collagen, the method comprising culturing the host cell comprising the polynucleotide under conditions suitable for expression of the porcine α1(III) collagen, and recovering the porcine α1(III) collagen from the host cell culture.

In certain embodiments, the present invention provides recombinant collagens and recombinant gelatins comprising porcine α1(III) collagen or fragments or variants thereof. The invention specifically provides for recombinant collagens and gelatins comprising SEQ ID NO: 12 or fragments or variants thereof.

Methods for producing recombinant animal collagens and gelatins are also provided. In one embodiment, the present invention provides a method for producing recombinant animal collagen, the method comprising introducing into a host cell at least one expression vector comprising a polynucleotide sequence encoding an animal collagen or procollagen, and at least one expression vector comprising a polynucleotide sequence encoding a post-translational enzyme, under conditions which permit the expression of the polynucleotides; and isolating the animal collagen. In a further aspect, the post-translational enzyme is selected from the group consisting of prolyl hydroxylase, peptidyl prolyl isomerase, collagen galactosyl hydroxylysyl glucosyl transferase, hydroxylysyl galactosyl transferase, C-proteinase, N-proteinase, lysyl hydroxylase, and lysyl oxidase. In one embodiment, the post-translational enzyme is selected from the same species as the animal collagen. In another embodiment, the host cell is selected from the same species as the animal collagen. In further embodiments, the host cell does not endogenously produce collagen, or does not endogenously produce a post-translational enzyme. A host cell comprising at least one expression vector encoding an animal and at least one expression vector encoding a post-translational enzyme is specifically provided.

In one aspect, the present invention provides a recombinant animal collagen of one type substantially free from collagen of any other type. Embodiments wherein the collagen of one type is specifically selected from the group consisting of type I, type II, type III, type IV, type V, type VI, type VII, type VIII, type IX, type X, type XI, type XII, type XIII, type XIV, type XV, type XVI, type XVII, type XVIII, type XIX, and type XX collagen are specifically contemplated.

Methods for producing recombinant animal gelatins are also provided. In one aspect, the method comprises providing recombinant animal collagen, and deriving recombinant animal gelatin therefrom. In another aspect, the method comprises producing recombinant animal gelatin directly from an altered animal collagen construct.

### BRIEF DESCRIPTION OF THE FIGURES

FIGS. 1A, 1B, and 1C show a nucleic acid sequence (SEQ NO: 1) encoding a bovine α1(I) collagen.

FIGS. 2A, 2B, 2C, and 2D show the amino acid sequence (SEQ ID NO: 2) of a bovine α1(I) collagen.

FIGS. 3A, 3B, and 3C show a nucleic acid sequence (SEQ ID NO: 3) encoding a bovine α1(III) collagen.

FIGS. 4A, 4B, 4C, and 4D show the amino acid sequence (SEQ ID NO: 4) of a bovine α1(III) collagen.

FIGS. 5A, 5B, and 5C show a nucleic acid sequence (SEQ ID NO: 5) encoding a bovine α1(III) collagen.

FIGS. 6A, 6B, 6C, and 6D show the amino acid sequence (SEQ ID NO: 6) of a bovine α1(III) collagen.

FIGS. 7A, 7B, and 7C show a nucleic acid sequence (SEQ ID NO: 7) encoding a porcine α1(I) collagen.

FIGS. 8A, 8B, 8C, and 8D show the amino acid sequence (SEQ ID NO: 8) encoding a porcine α1(I) collagen.

FIGS. 9A, 9B, and 9C show a nucleic acid sequence (SEQ ID NO: 9) encoding a porcine α2(I) collagen.

FIGS. 10A, 10B, and 10C show the amino acid sequence (SEQ ID NO: 10) of a porcine α2(I) collagen.

FIGS. 11A, 11B, and 11C show a nucleic acid sequence (SEQ ID NO: 11) encoding a porcine α1(III) collagen.

FIGS. 12A, 12B, and 12C show the amino acid sequence (SEQ ID NO: 12) of a porcine α1(III) collagen.

FIGS. 13A, 13B, 13C, 13D, 13E, 13F, 13G, 13H, and 13I depict the translated bovine α1(I) collagen open reading frame sequences aligned with known human (HU), mouse (MUS), dog (CANIS), bullfrog (RANA), and Japanese newt (CYNPS) collagen sequences.

### DETAILED DESCRIPTION OF THE INVENTION

Before the present proteins, nucleotide sequences, and methods are described, it is understood that this invention is not limited to the particular methodology, protocols, cell lines, vectors, and reagents described, as these may vary. It is also to be understood that the terminology used herein is for the purpose of describing particular embodiments only, and is not intended to limit the scope of the present invention.

It must be noted that as used herein, and in the appended claims, the singular forms “a,” “an,” and “the” include plural reference unless the context clearly dictates otherwise. Thus, for example, reference to “a host cell” is reference to one or more of such host cells and equivalents thereof known to those skilled in the art, and reference to “an antibody” is a reference to one or more antibodies and equivalents thereof known to those skilled in the art, and so forth.

Unless defined otherwise, all technical and scientific terms used herein have the meanings as commonly understood by one of ordinary skill in the art to which the invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies, etc., which are reported in the publications which might be used in connection with the invention. Nothing herein is to be construed as an admission that the invention is not entitled to antedate such disclosure by virtue of prior invention. Each reference cited herein is incorporated herein by reference in its entirety.

The practice of the present invention will employ, unless otherwise indicated, conventional methods of chemistry, biochemistry, molecular biology, immunology and pharmacology, within the skill of the art. Such techniques are explained fully in the literature. See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

### DEFINITIONS

The term “collagen” refers to any one of the known collagen types, including collagen types I through XX, as well as to any other collagens, whether natural, synthetic, semi-synthetic, or recombinant. The term also encompasses procollagens. The term collagen encompasses any single-chain polypeptide encoded by a single polynucleotide, as well as homotrimeric and heterotrimeric assemblies of collagen chains. The term “collagen” specifically encompasses variants and fragments thereof, and functional equivalents and derivatives thereof, which preferably retain at least one structural or functional characteristic of collagen, for example, a (Gly-X—Y)ₙ domain.

So, for example, the term “bovine α1(I) collagen” refers to a single-chain bovine α1(I) collagen encoded by a single polynucleotide sequence, and to any corresponding procollagen, or to any fragment, variant, functional equivalent, or derivative thereof. The term “bovine type I collagen” refers to a homotrimeric or heterotrimeric collagen comprising bovine type I collagen chains, and to any corresponding procollagen, or to any fragment, variant, functional equivalent, or derivative thereof.

The term “procollagen” refers to a procollagen corresponding to any one of the collagen types I through XX, as well as to a procollagen corresponding to any other collagens, whether natural, synthetic, semi-synthetic, or recombinant, that possesses additional C-terminal and/or N-terminal propeptides or telopeptides that assist in trimer assembly, solubility, purification, or any other function, and that then are subsequently cleaved by N-proteinase, C-proteinase, or other enzymes, e.g., proteolytic enzymes, associated with collagen production. The term procollagen specifically encompasses variants and fragments thereof, and functional equivalents and derivatives thereof, which preferably retain at least one structural or functional characteristic of collagen, for example, a (Gly-X—Y)ₙ domain.

The term “bovine α1(I)” refers to a bovine α1(I) collagen or functional equivalent thereof, and to fragments and variants thereof, and to polynucleotides encoding such polypeptides from any source whether natural, synthetic, semi-synthetic, or recombinant.

The term “bovine α1(III)” refers to a bovine α1(III) collagen or functional equivalent thereof, to fragments and variants thereof, and to polynucleotides encoding such polypeptides from any source whether natural, synthetic, semi-synthetic, or recombinant.

The term “porcine α1(I)” refers to a porcine α1(I) collagen or functional equivalent thereof, to fragments and variants thereof, and to polynucleotides encoding such polypeptides from any source whether natural, synthetic, semi-synthetic, or recombinant.

The term “porcine α2(I)” refers to a porcine α2(I) collagen or functional equivalent thereof, to fragments and variants thereof, and to polynucleotides encoding such polypeptides from any source whether natural, synthetic, semi-synthetic, or recombinant.

The term “porcine α1(III)” refers to a porcine α1(III) collagen or functional equivalent thereof, to fragments and variants thereof, and to polynucleotides encoding such polypeptides from any source whether natural, synthetic, semi-synthetic, or recombinant.

“Gelatin” as used herein refers to any gelatin, whether extracted by traditional methods or recombinant or biosynthetic in origin, or to any molecule having at least one structural and/or functional characteristic of gelatin. Gelatin is currently obtained by extraction from collagen derived from animal (e.g., bovine, porcine, rodent, chicken, equine, piscine) sources, e.g., bones and tissues. The term gelatin encompasses both the composition of more than one polypeptide included in a gelatin product, as well as an individual polypeptide contributing to the gelatin material. Thus, the term recombinant gelatin as used in reference to the present invention encompasses both a recombinant gelatin material comprising the present gelatin polypeptides, as well as an individual gelatin polypeptide of the present invention.

Polypeptides from which gelatin can be derived are polypeptides such as collagens, procollagens, and other polypeptides having at least one structural and/or functional characteristic of collagen. Such a polypeptide could include a single collagen chain, or a collagen homotrimer or heterotrimer, or any fragments, derivatives, oligomers, polymers, or subunits thereof, containing at least one collagenous domain (a Gly-X—Y region). The term specifically contemplates engineered sequences not found in nature, such as altered collagen constructs, etc. An altered collagen construct is a polynucleotide comprising a sequence that is altered, through deletions, additions, substitutions, or other changes, from the naturally occurring collagen gene.

An “adjuvant” is any agent added to a drug or vaccine to increase, improve, or otherwise aid its effect. An adjuvant used in a vaccine formulation might be an immunological agent that improves the immune response by producing a non-specific stimulator of the immune response. Adjuvants are often used in non-living vaccines.

The terms “allele” or “allelic sequence” refer to alternative forms of genetic sequences. Alleles may result from at least one mutation in the nucleic acid sequence and may result in altered mRNAs or polypeptides whose structure or function may or may not be altered. Any given natural or recombinant gene may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

“Altered” polynucleotide sequences include those with deletions, insertions, or substitutions of different nucleotides resulting in a polynucleotide that encodes the same or a functionally equivalent polypeptide. Included within this definition are sequences displaying polymorphisms that may or may not be readily detectable using particular oligonucleotide probes or through deletion of improper or unexpected hybridization to alleles, with a locus other than the normal chromosomal locus for the subject polynucleotide sequence.

“Altered” polypeptides may contain deletions, insertions, or substitutions of amino acid residues which produce a silent change and result in a functionally equivalent polypeptide. Deliberate amino acid substitutions may be made on the basis of similarity in polarity, charge, solubility, hydrophobicity, hydrophilicity, and/or the amphipathic nature of the residues as long as the biological or immunological activity of the encoded polypeptide is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine, glycine and alanine, asparagine and glutamine, serine and threonine, and phenylalanine and tyrosine.

“Amino acid” or “polypeptide” sequences or “polypeptides,” as these terms are used herein, refer to oligopeptide, peptide, polypeptide, or protein sequences, and fragments thereof, and to naturally occurring or synthetic molecules. Polypeptide or amino acid fragments are any portion of a polypeptide which retains at least one structural and/or functional characteristic of the polypeptide. In at least one embodiment of the present invention, polypeptide fragments are those retaining at least one (Gly-X—Y)ₙ region.

The term “animal” as it is used in reference, for example, to “animal collagens” encompasses any collagens, whether natural, synthetic, semi-synthetic, or recombinant. Animal sources include, for example, mammalian sources, including, but not limited to, bovine, porcine, equine, rodent, and ovine sources, and other animal sources, including, but not limited to, chicken and piscine sources, and non-vertebrate sources.

“Antigenicity” relates to the ability of a substance to, when introduced into the body, stimulate the immune response and the production of an antibody. An agent displaying the property of antigenicity is referred to as being antigenic. Antigenic agents can include, but are not limited to, a variety of macromolecules such as, for example, proteins, lipoproteins, polysaccharides, nucleic acids, bacteria and bacterial components, and viruses and viral components.

The terms “complementary” or “complementarity,” as used herein, refer to the natural binding of polynucleotides by base-pairing. For example, the sequence “A-G-T” binds to the complementary sequence “T-C-A.” Complementarity between two single-stranded molecules may be “partial,” when only some of the nucleic acids bind, or may be complete, when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands, and in the design and use, for example, of peptide nucleic acid (PNA) molecules.

A “deletion” is a change in an amino acid or nucleotide sequence that results in the absence of one or more amino acid residues or nucleotides.

The term “derivative,” as applied to polynueleotides, refers to the chemical modification of a polynucleotide encoding a particular polypeptide or complementary to a polynucleotide encoding a particular polypeptide. Such modifications include, for example, replacement of hydrogen by an alky, acyl, or amino group. As used herein to refer to polypeptides, the term “derivative” refers to a polypeptide which is modified, for example, by hydroxylation, glycosylation, pegylation, or by any similar process. The term “derivatives” encompasses those molecules containing at least one structural and/or functional characteristic of the molecule from which it is derived.

A molecule is said to be a “chemical derivative” of another molecule when it contains additional chemical moieties not normally a part of the molecule. Such moieties can improve the molecule's solubility, absorption, biological half-life, and the like. The moieties can alternatively decrease the toxicity of the molecule, eliminate or attenuate any undesirable side effect of the molecule, and the like. Moieties capable of mediating such effects are generally available in the art and can be found for example, in Remington's Pharmaceutical Sciences, supra. Procedures for coupling such moieties to a molecule are well known in the art.

An “excipient” as the term is used herein is any inert substance used as a diluent or vehicle in the formulation of a drug, a vaccine, or other pharmaceutical composition, in order to confer a suitable consistency or form to the drug, vaccine, or pharmaceutical composition.

The term “functional equivalent” as it is used herein refers to a polypeptide or polynucleotide that possesses at least one functional and/or structural characteristic of a particular polypeptide or polynucleotide. A functional equivalent may contain modifications that enable the performance of a specific function. The term “functional equivalent” is intended to include fragments, mutants, hybrids, variants, analogs, or chemical derivatives of a molecule.

A “fusion protein” is a protein in which peptide sequences from different proteins are operably linked.

The term “hybridization” refers to the process by which a nucleic acid sequence binds to a complementary sequence through base pairing. Hybridization conditions can be defined by, for example, the concentrations of salt or formamide in the prehybridization and hybridization solutions, or by the hybridization temperature, and are well known in the art. Hybridization can occur under conditions of various stringency.

In particular, stringency can be increased by reducing the concentration of salt, increasing the concentration of formamide, or raising the hybridization temperature. For example, for purposes of the present invention, hybridization under high stringency conditions occurs in about 50% formamide at about 37° C. to 42° C., and under reduced stringency conditions in about 35% to 25% formamide at about 30° C. to 35° C. In particular, hybridization occurs in conditions of highest stringency at 42° C. in 50% formamide, 5×SSPE, 0.3% SDS, and 200 μg/ml sheared and denatured salmon sperm DNA.

The temperature range corresponding to a particular level of stringency can be further narrowed by methods known in the art, for example, by calculating the purine to pyrimidine ratio of the nucleic acid of interest and adjusting the temperature accordingly. To remove nonspecific signals, blots can be sequentially washed, for example, at room temperature under increasingly stringent conditions of up to 0.1×SSC and 0.5% SDS. Variations on the above ranges and conditions are well known in the art.

“Immunogenicity” relates to the ability to evoke an immune response within an organism. An agent displaying the property of immunogenicity is referred to as being immunogenic. Agents can include, but are not limited to, a variety of macromolecules such as, for example, proteins, lipoproteins, polysaccharides, nucleic acids, bacteria and bacterial components, and viruses and viral components. Immunogenic agents often have a fairly high molecular weight (usually greater than 10 kDa).

“Infectivity” refers to the ability to be infective or the ability to produce infection, referring to the invasion and multiplication of microorganisms, such as bacteria or viruses within the body.

The terms “insertion” or “addition” refer to a change in a polypeptide or polynucleotide sequence resulting in the addition of one or more amino acid residues or nucleotides, respectively, as compared to the naturally occurring molecule.

The term “isolated” as used herein refers to a molecule separated not only from proteins, etc., that are present in the natural source of the protein, but also from other components in general, and preferably refers to a molecule found in the presence of, if anything, only a solvent, buffer, ion, or other component normally present in a solution of the same. As used herein, the terms “isolated” and “purified” do not encompass molecules present in their natural source.

The term “microarray” refers to any arrangement of nucleic acids, amino acids, antibodies, etc., on a substrate. The substrate can be any suitable support, e.g., beads, glass, paper, nitrocellulose, nylon, or any appropriate membrane, etc. A substrate can be any rigid or semi-rigid support including, but not limited to, membranes, filters, wafers, chips, slides, fibers, beads, including magnetic or nonmagnetic beads, gels, tubing, plates, polymers, microparticles, capillaries, etc. The substrate can provide a surface for coating and/or can have a variety of surface forms, such as wells, pins, trenches, channels, and pores, to which the nucleic acids, amino acids, etc., may be bound.

The term “microorganism” can include, but is not limited to, viruses, bacteria, Chlamydia, rickettsias, mycoplasmas, ureaplasmas, fungi, and parasites, including infectious parasites such as protozoans.

The terms “nucleic acid” or “polynucleotide” sequences or “polynucleotides” refer to oligonucleotides, nucleotides, or polynucleotides, or any fragments thereof, and to DNA or RNA of natural or synthetic origin which may be single- or double-stranded and may represent the sense or antisense strand, to peptide nucleic acid (PNA), or to any DNA-like or RNA-like material, natural or synthetic in origin. Polynucleotide fragments are any portion of a polynucleotide sequence that retains at least one structural or functional characteristic of the polynucleotide. In one embodiment of the present invention, polynucleotide fragments are those that encode at least one (Gly-X—Y)ₙ region. Polynucleotide fragments can be of variable length, for example, greater than 60 nucleotides in length, at least 100 nucleotides in length, at least 1000 nucleotides in length, or at least 10,000 nucleotides in length.

The phrase “percent similarity” (% similarity) refers to the percentage of sequence similarity found in a comparison of two or more polypeptide or polynucleotide sequences. Percent similarity can be determined by methods well-known in the art. For example, percent similarity between amino acid sequences can be calculated using the Clustal method. (See, e.g., Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent similarity can be calculated by other methods known in the art, for example, by varying hybridization conditions, and can be calculated electronically using programs such as the MEGALIGN program (DNASTAR Inc., Madison, Wis.).

As used herein, the term “plant” includes reference to one or more plants, i.e., any eukaryotic autotrophic organisms, such as angiosperms and gymnosperms, monotyledons and dicotyledons, etc., including, but not limited to, soybean, cotton, alfalfa, flax, tomato, sugar, beet, sunflower, potato, tobacco, maize, wheat, rice, lettuce, banana, cassava, safflower, oilseed, rape, mustard, canola, hemp, algae, kelp, etc. The term “plant” also encompasses one or more plant cells. The term “plant cells” includes, but is not limited to, vegetative tissues and organs such as seeds, suspension cultures, embryos, meristematic regions, callus tissue, leaves, roots, shoots, gametophytes, sporophytes, pollen, tubers, corms, bulbs, flowers, fruits, cones, microspores, etc.

The term “post-translational enzyme” refers to any enzyme that catalyzes post-translational modification of, for example, any collagen or procollagen. The term encompasses, but is not limited to, for example, prolyl hydroxylase, peptidyl prolyl isomerase, collagen galactosyl hydroxylysyl glucosyl transferase, hydroxylysyl galactosyl transferase, C-proteinase, N-proteinase, lysyl hydroxylase, and lysyl oxidase.

As used herein, the term “promoter” generally refers to a regulatory region of nucleic acid sequence capable of initiating, directing, and mediating the transcription of a polynucleotide sequence. Promoters may additionally comprise recognition sequences, such as upstream or downstream promoter elements, which may influence the transcription rate.

The term “non-constitutive promoters” refers to promoters that induce transcription via a specific tissue, or may be otherwise under environmental or developmental controls, and includes repressible and inducible promoters such as tissue-preferred, tissue-specific, and cell type-specific promoters. Such promoters include, but are not limited to, the AdH1 promoter, inducible by hypoxia or cold stress, the Hsp70 promoter, inducible by heat stress, and the PPDK promoter, inducible by light.

Promoters which are “tissue-preferred” are promoters that preferentially initiate transcription in certain tissues. Promoters which are “tissue-specific” are promoters that initiate transcription only in certain tissues. “Cell type-specific” promoters are promoters which primarily drive expression in certain cell types in at least one organ, for example, vascular cells.

“Inducible” or “repressible” promoters are those under control of the environment, such that transcription is effected, for example, by an environmental condition such as anaerobic conditions, the presence of light, biotic stresses, etc., or in response to internal, chemical, or biological signals, e.g., glyceraldehyde phosphate dehydrogenase, AOX1 and AOX2 methanol-inducible promoters, or to physical damage.

As used herein, the term “constitutive promoters” refers to promoters that initiate, direct, or mediate transcription, and are active under most environmental conditions and states of development or cell differentiation. Examples of constitutive promoters, include, but are not limited to, the cauliflower mosaic virus (CaMv) 35S, the 1′- or 2′-promoter derived from T-DNA of Agrobacteriuam tumefaciens, the ubiquitin 1 promoter, the Smas promoter, the cinnamyl alcohol dehydrogenase promoter, glyceraldehyde dehydrogenase promoter, and the Nos promoter, etc.

The term “purified” as it is used herein denotes that the indicated molecule is present in the substantial absence of other biological macromolecules, e.g., polynucleotides, proteins, and the like. The term preferably contemplates that the molecule of interest is present in a solution or composition at least 80% by weight; preferably, at least 85% by weight; more preferably, at least 95% by weight; and, most preferably, at least 99.8% by weight. Water, buffers, and other small molecules, especially molecules having a molecular weight of less than about one kDa, can be present.

The term “substantially purified”, as used herein, refers to nucleic or amino acid sequences that are removed from their natural environment, isolated or separated, and are at least 60% free, preferably 75% free, and most preferably 90% free from other components with which they are naturally associated.

A “substitution” is the replacement of one or more amino acids or nucleotides by different amino acids or nucleotides, respectively.

The term “transfection” as used herein refers to the process of introducing an expression vector into a cell. Various transfection techniques are known in the art, for example, microinjection, lipofection, or the use of a gene gun.

“Transformation”, as defined herein, describes a process by which exogenous nucleic acid sequences, e.g., DNA, enters and changes a recipient cell. Transformation may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and particle bombardment. Such “transformed” cells include stably transformed cells in which the inserted DNA is capable of replication either as an autonomously replicating plasmid or as part of the host chromosome, and also include cells which transiently express the inserted nucleic acid for limited periods of time.

As used herein, the term “vaccine” refers to a preparation of killed or modified microorganisms, living attenuated organisms, or living fully virulent organisms, or any other agent, including, but not limited to peptides, proteins, biological macromolecules, or nucleic acids, natural, synthetic, or semi-synthetic, administered to produce or artificially increase immunity to a particular disease, in order to prevent future infection with a similar entity. Vaccines can be live or inactivated microorganisms or agents, including viruses and bacteria, as well as subunit, synthetic, semi-synthetic, or recombinant DNA-based.

Vaccines can be monovalent (a single strain/microorganism/disease vaccine) consisting of one microorganism or agent (e.g., poliovirus vaccine) or the antigens of one microorganism or agent. Vaccines can also be multivalent, e.g., divalent, trivalent, etc. (a combined vaccine), consisting of more than one microorganism or agent (e.g., a measles-mumps-rubella (MMR) vaccine) or the antigens of more than one microorganism or agent.

Live vaccines are prepared from living microorganisms. Attenuated vaccines are live vaccines prepared from microorganisms which have undergone physical alteration (such as radiation or temperature conditioning) or serial passage in laboratory animal hosts or infected tissue/cell cultures, such treatments producing avirulent strains or strains of reduced virulence, but maintaining the capability of inducing protective immunity. Examples of live attenuated vaccines include measles, mumps, rubella, and canine distemper. Inactivated vaccines are vaccines in which the infectious microbial components have been destroyed, e.g., by chemical or physical treatment (such as formalin, beta-propiolactone, or gamma radiation), without affecting the antigenicity or immunogenicity of the viral coat or bacterial outer membrane proteins. Examples of inactivated or subunit vaccines include influenza, Hepatitis A, and poliomyelitis (IPV) vaccines.

Subunit vaccines are composed of key macromolecules from, e.g., the viral, bacterial, or other agent responsible for eliciting an immune response. These components can be obtained in a number of ways, for example, through purification from microorganisms, generation using recombinant DNA technology, etc. Subunit vaccines can contain synthetic mimics of any infective agent. Subunit vaccines can include macromolecules such as bacterial protein toxins (e.g., tetanus, diphtheria), viral proteins (e.g., from influenza virus), polysaccharides from encapsulated bacteria (e.g., from Haemophilus influenzae and Streptococcus pneumonia), and viruslike particles produced by recombinant DNA technology (e.g., hepatitis B surface antigen), etc.

Synthetic vaccines are vaccines made up of small synthetic peptides that mimic the surface antigens of pathogens and are immunogenic, or may be vaccines manufactured with the aid of recombinant DNA techniques, including whole viruses whose nucleic acids have been modified.

Semi-synthetic vaccines, or conjugate vaccines, consist of polysaccharide antigens from microorganisms attached to protein carrier molecules.

DNA vaccines contain recombinant DNA vectors encoding antigens, which, upon expression of the encoded antigen in host cells having taken up the DNA, induce humoral and cellular immune responses against the encoded antigens.

Vaccines have been developed for a variety of infectious agents. The present invention is directed to recombinant gelatins that can be used in vaccine formulations regardless of the agent involved, and are thus not limited to use in the vaccines specifically described herein by way of example. Vaccines include, but are not limited to, vaccines for vacinnia virus (small pox), polio virus (Salk and Sabin), mumps, measles, rubella, diphtheria, tetanus, Varicella-Zoster (chicken pox/shingles), pertussis (whopping cough), Bacille Calmette-Guerin (BCG, tuberculosis), haemophilus influenzae meningitis, rabies, cholera, Japanese encephalitis virus, salmonella typhi, shigella, hepatitis A, hepatitis B, adenovirus, yellow fever, foot-and-mouth disease, herpes simplex virus, respiratory syncytial virus, rotavirus, Dengue, West Nile virus, Turkey herpes virus (Marek's Disease), influenza, and anthrax. The term vaccine as used herein includes reference to vaccines to various infectious and autoimmune diseases and cancers that have been or that will be developed, for example, vaccines to various infectious and autoimmune diseases and cancers, e.g., vaccines to HIV, HCV, malaria, and vaccines to breast, lung, colon, renal, bladder, and ovarian cancers.

A polypeptide or amino acid “variant” is an amino acid sequence that is altered by one or more amino acids from a particular amino acid sequence. A polypeptide variant may have conservative changes, wherein a substituted amino acid has similar structural or chemical properties to the amino acid replaced, e.g., replacement of leucine with isoleucine. A variant may also have nonconservative changes, in which the substituted amino acid has physical properties different from those of the replaced amino acid, e.g., replacement of a glycine with a tryptophan. Analogous minor variations may also include amino acid deletions or insertions, or both. Preferably, amino acid variants retain certain structural or functional characteristics of a particular polypeptide. Guidance in determining which amino acid residues may be substituted, inserted, or deleted may be found, for example, using computer programs well known in the art, such as LASERGENE software (DNASTAR Inc., Madison, Wis.).

A polynucleotide variant is a variant of a particular polynucleotide sequence that preferably has at least about 80%, more preferably at least about 90%, and most preferably at least about 95% polynucleotide sequence similarity to the particular polynucleotide sequence. It will be appreciated by those skilled in the art that as a result of the degeneracy of the genetic code, a multitude of variant polynucleotide sequences encoding a particular protein, some bearing minimal homology to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard codon triplet genetic code, and all such variations are to be considered as being specifically disclosed.

### Invention

The present invention provides for the production of recombinant animal collagens and gelatins. These animal collagens and gelatins provide advantages over currently available materials in that they are produced as well-characterized and pure proteins. Methods for producing these animal collagens and gelatins are also provided. In certain embodiments, the present invention provides animal collagens and gelatins derived from bovine type I collagen, bovine type III collagen, porcine type I collagen, and porcine type III collagen. In specific embodiments, bovine α1(I), bovine α1(III), porcine α1(I), porcine α2(I), and porcine α1(III) collagens and gelatins are provided.

The present invention provides for production of relatively large amounts of single types of animal collagen, synthesized in recombinant cell culture systems that do not make any other collagen types. For example, the present invention provides animal collagen type I that is substantially free from any other collagen type. Using methods of the present invention, purification of collagen is greatly facilitated.

The present invention is further directed to vectors and plasmids used in the methods of the invention. These vectors and/or plasmids are comprised of a polynucleotide encoding the desired collagen, or fragments or variants thereof, necessary promoters, and other sequences necessary for the proper expression of such polypeptides. The polynucleotide encoding a collagen is preferably obtained from animal sources. Animal sources include non-human mammalian sources, such as bovine, ovine, and porcine sources. In one embodiment, the vectors and plasmids of the present invention further include at least one polynucleotide encoding one or more post-translational enzymes or functional equivalents thereof. The polynucleotide encoding one or more post-translational enzymes may be derived from any of the above-mentioned species. In a preferred embodiment, the collagen-encoding polynucleotide is derived from the same species as the polynucleotide encoding the post-translational enzyme.

In a further embodiment, at least one polynucleotide encoding a post-translational enzyme, such as prolyl 4-hydroxylase, C-proteinase, N-proteinase, lysyl oxidase, or lysyl hydroxylase, is inserted into cells that do not naturally produce post-translational enzymes, such as yeast cells, or may not naturally produce sufficient amounts of post-translational enzymes, such as some mammalian and insect cells. In a preferred embodiment of the present invention, the post-translational enzyme is prolyl 4-hydroxylase, wherein the polynucleotides encoding an α subunit of prolyl 4-hydroxylase and the polynucleotides encoding a β subunit of prolyl 4-hydroxylase are inserted into a cell to produce a biologically active prolyl 4-hydroxylase enzyme.

The present invention specifically contemplates the use of any compound, biological or chemical, that confers hydroxylation, e.g., proline hydroxylation and/or lysine hydroxylation, etc., as desired, to the present recombinant animal collagens and gelatins. This includes, for example, prolyl 4-hydroxylase from any species, endogenously or exogenously supplied, including various isoforms of prolyl 4-hydroxylase and any variants or fragments or subunits of prolyl 4-hydroxylase having the desired activity, whether native, synthetic, or semi-synthetic, and other hydroxylases such as prolyl 3-hydroxylase, etc. (See, e.g., U.S. Pat. No. 5,928,922), incorporated by reference herein in its entirety.) In one embodiment, the prolyl hydroxylase activity is conferred by a prolyl hydroxylase derived from the same species as the polynucleotide encoding recombinant collagen or gelatin, or encoding a polypeptide from which recombinant gelatin can be derived. In a further embodiment, the prolyl 4-hydroxylase is from an animal and the encoding polynucleotide is derived from sequence from the same animal.

The present invention provides a method for producing recombinant animal collagens and gelatins. It is to be noted that while, for clarity, the present methods of production are directed generally to the production of collagens, the production methods can be applied to the production of gelatins directly from altered collagen constructs, and the production of polypeptides from which gelatins can be derived. In one embodiment, the method comprises introducing into a host cell, under conditions suitable for expression, an expression vector encoding an animal collagen or procollagen, or fragments or variants thereof, and a second expression vector encoding a post-translational enzyme, and isolating the collagen. In a preferred embodiment, the post translational enzyme is prolyl hydroxylase. (See, e.g., U.S. Pat. No. 5,593,859, incorporated by reference herein in its entirety.)

The present invention further provides animal collagens comprising at least one animal collagen chain or subunit, or fragment or variants thereof. In a preferred embodiment, the collagen composition of the present invention comprises a collagen chain, or fragment or variant thereof, that is comprised of a structural amino acid pattern of (Gly-X—Y)ₙ, wherein X and Y can be any amino acid. Preferably, the amino acids of X and/or Y are either proline or hydyroxyproline; glycine (Gly) is in every third residue position of each chain; and the number of repeating Gly-X—Y triplets is of about 10-3000 (i.e., n=10-3000). The Gly-X—Y unit within a collagen chain, or subunit or fragment thereof, is the same or different. In one aspect, the collagen compositions of the present invention are less than fully glycosolated or less than fully hydroxylated. For example, the collagen of the present invention may be deglycosolated, unglycosolated, partially glycosolated, and partially hydroxylated. In a further aspect of the present invention, the collagen compositions are comprised of one type of collagen, and are substantially free from any other type of collagen. In one embodiment, the present invention provides, a recombinant collagen type I composition substantially free from any other collagen, e.g., of types II through XX, etc.

The invention further comprises recombinant polypeptides, including fusion products produced from chimeric genes wherein, for example, relevant epitopes of collagen can be manufactured for therapeutic and other uses. Furthermore, the present invention encompasses any modifications made to the collagens or gelatins or compositions thereof or any degradation products thereof. Such modifications include, for example, processing of animal collagens or collagenous proteins and gelatin.

The present invention further provides gelatin compositions. Specifically, the present invention provides gelatin compositions derived from animal collagens. In various embodiments, the gelatin composition is derived from bovine, porcine, or piscine collagen. In another aspect of the present invention, the composition is composed of a gelatin derived from a collagen type substantially free from any other collagen type. In a further aspect of the present invention, the gelatin composition is comprised of denatured triple helices, and includes at least one collagen subunit or chain, or fragment or variant thereof.

The present invention further provides methods of producing a gelatin by expressing collagen or functional equivalents thereof, and deriving gelatin therefrom. The present invention further provides for direct expression of recombinant animal gelatin from an altered animal collagen construct. (See, e.g., commonly owned, co-pending application U.S. application Ser. No. 09/710,239, entitled “Recombinant Gelatins,” filed Nov. 10, 2000, and incorporated herein by reference in its entirety.) More specifically, the process involves inserting into a cell an expression vector comprising at least one polynucleotide encoding an animal collagen, or fragments or variants thereof, and an expression vector comprising at least one polynucleotide encoding a collagen post-translational enzyme or subunit thereof, recovering the collagen, and deriving gelatin from the collagen.

In some embodiments of the present invention, the gelatin compositions may be obtained directly from the isolated collagen or from biomass or culture media. Methods, processes, and techniques of producing gelatin compositions from collagen include denaturing the triple helical structure of the collagen utilizing detergents, heat or denaturing agents. Additionally, these methods, processes, and techniques include, but are not limited to, treatments with strong alkali or strong acids, heat extraction in aqueous solution, ion exchange chromatography, cross-flow filtration and heat drying, and other methods known in the art that may be applied to collagen to produce the gelatin compositions. The same methods, processes, and techniques may be applied to biomass or culture media to produce the gelatin compositions of the present invention.

The present invention further relates to various animal collagens. In one aspect, the present invention provides a bovine type I collagen and a bovine type III collagen. In specific embodiments, a bovine α1(I) collagen and a bovine α1(III) collagen and fragments and variants thereof are provided.

In another aspect, the present invention provides porcine type I and porcine type III collagens. In addition, the present invention provides a porcine α1(I) collagen, a porcine α2(I) collagen, and a porcine α1(III) collagen, and fragments and variants thereof.

The present invention also provides polynucleotides encoding bovine α1(I) collagen, bovine α1(III) collagen, porcine α1(I) collagen, or a porcine α1(III) collagen, or porcine α2(I) collagen, or fragments or variants thereof. The invention further provides polynucleotides complementary to the encoding polynucleotides, as well as polynucleotides that hybridize, under stringent conditions, to these nucleic acid sequences. The present invention also provides methods of producing recombinant bovine type I collagens, bovine type III collagens, porcine type I collagens, or porcine type III collagens or fragments or variants thereof.

In another aspect of the present invention, the expression vectors comprising the polynucleotides of the present invention may be inserted into host cells to produce animal collagens or gelatins, for example, bovine type I, bovine type III, porcine type I, and porcine type III collagens or gelatins. In one method, an expression vector comprising a polynucleotide of the present invention is co-expressed in host cells with an expression vector comprising a polynucleotide encoding a polypeptide of the present invention with an expression vector comprising a polynucleotide encoding a post-translational enzyme. In one embodiment, the post-translational enzyme is prolyl 4-hydroxylase, comprising an a subunit and a β subunit.

The recombinant animal collagens and gelatins of the present invention limit human exposure to various contaminants that may be present in animal tissues currently used as raw material in the manufacture of collagens and collagen-derived materials such as gelatin. Moreover, the collagens and gelatins of the present invention are more reproducible than collagens or gelatins currently obtained from raw animal sources.

In accordance with the invention, encoding polynucleotide sequences, as well as being well-characterized proteins with predictable performance may be used to generate recombinant molecules that direct the expression of the present polypeptides in appropriate host cells.

Nucleic acid sequences encoding collagens have been generally described in the art. (See, e.g., Fuller and Boedtker (1981) Biochemistry 20:996-1006; Sandell et al. (1984) J Biol Chem. 259:7826-34; Kohno et al. (1984) J Biol Chem. 259:13668-13673; French et al. (1985) Gene 39:311-312; Metsaranta et al. (1991) J Biol Chem. 266:16862-16869; Metsaranta et al, (1991) Biochim Biophys Acta 1089:241-243; Wood et al. (1987) Gene 61:225-230; Glumoff et al. (1994) Biochim Biophys Acta 1217:41-48; Shirai et al. (1998) Matrix Biology 17:85-88; Tromp et al. (1988) Biochem J. 253:919-912; Kuivaniemi et al. (1988) Biochem J. 252:633-640; and Ala-Kokko et al. (1989) Biochem J. 260:509-516.)

In one embodiment, the present invention provides a polynucleotide sequence comprising an isolated and purified polynucleotide sequence having greater than 70% similarity to the bovine α1(I) collagen polynucleotide sequence present in SEQ ID NO: 1, or fragments or variants thereof, preferably greater than 80% similarity, and more preferably greater than 90% similarity. In a further embodiment, the polynucleotide sequence encodes the bovine α1(I) collagen amino acid sequence of SEQ ID NO: 2, or fragments or variants thereof.

In another embodiment, the polynucleotide sequence of the present invention comprises an isolated and purified polynucleotide sequence having greater than 70% similarity to the bovine α1(III) collagen polynucleotide sequence of SEQ ID NO: 3 or of SEQ ID NO: 5, or fragments or variants thereof, preferably greater than 80% similarity, and more preferably greater than 90% similarity. In one embodiment, the polynucleotide sequence encodes the bovine α1(III) sequence of SEQ ID NO: 4 or of SEQ ID NO: 6, or fragments or variants thereof.

In one aspect, the present invention provides an isolated and purified polynucleotide sequence comprising a polynucleotide having greater than 70% similarity to the porcine α1(I) collagen polynucleotide sequence present in SEQ ID NO: 7, or fragments or variants thereof, preferably greater than 80% similarity, and more preferably greater than 90% similarity. In one embodiment, the polynucleotide encodes the amino acid sequence of SEQ ID NO: 8, or fragments or variants thereof.

In another aspect, the present invention contemplates an isolated and purified polynucleotide sequence comprising a sequence with greater than 70% similarity to the porcine α2(I) collagen polynucleotide sequence present in SEQ ID NO: 9, or fragments or variants thereof, preferably greater than 80% similarity, and more preferably greater than 90% similarity. In one embodiment, the polynucleotide sequence encodes the porcine α2(I) amino acid sequence of SEQ ID NO: 10, or fragments or variants thereof.

In a further aspect, the present invention relates to an isolated and purified polynucleotide sequence having greater than 70% similarity to the porcine α1(III) collagen polynucleotide sequence present in SEQ ID NO: 11, or fragments or variants thereof, preferably greater than 80% similarity, or more preferably greater than 90% similarity. In another preferred embodiment, the polynucleotide encodes the porcine α1(III) collagen amino acid sequence present in SEQ ID NO: 12, or fragments or variants thereof.

Collagens from which nucleic acid sequence is not available may be obtained, by various methods known in the art, from cDNA libraries prepared from tissues believed to possess the type of collagen of interest and to express that collagen at a detectable level. For example, a cDNA library could be constructed by obtaining polyadenylated mRNA from a cell line known to express the novel collagen, or a cDNA library previously made to the tissue/cell type could be used. The cDNA library is screened with appropriate nucleic acid probes, and/or the library is screened with suitable polyclonal or monoclonal antibodies that specifically recognize other collagens. Appropriate nucleic acid probes include oligonucleotide probes that encode known portions of the novel collagen from the same or different species. Other suitable probes include, without limitation, oligonucleotides, cDNAs, or fragments thereof that encode the same or similar gene, and/or homologous genomic DNAs or fragments thereof. Screening the cDNA or genomic library with the selected probe may be accomplished using standard procedures known to those in the art. (See, e.g., Maniatis et al., supra.). Other means for identifying novel collagens involve known techniques of recombinant DNA technology, such as by direct expression cloning or using the polymerase chain reaction (PCR) as described in U.S. Pat. No. 4,683,195, or in, e.g., Maniatis et al., supra, or Ausubel et al., supra.

Altered polynucleotide sequences which may be used in accordance with the invention include deletions, additions, or substitutions of different nucleotide residues resulting in a sequence that encodes the same or a functionally equivalent gene product. The gene product itself may contain deletions, additions, or substitutions of amino acid residues still resulting in a functionally equivalent polypeptide.

The nucleic acid sequences of the invention may be engineered in order to alter the coding sequence for a variety of ends including, but not limited to, alterations which modify processing and expression of the gene product. For example, alternative secretory signals may be substituted for the native secretory signal and/or mutations may be introduced using techniques which are well known in the art, e.g., site-directed mutagenesis, to insert new restriction sites, to alter glycosylation patterns, phosphorylation, etc. In one embodiment, the polynucleotides of the present invention are modified in the silent position of any triplet amino acid codon so as to better conform to the codon preference of the particular host organism.

The polynucleotides of the present invention are further directed to sequences which encode variants and fragments of the described animal collagens and gelatins. These amino acid fragments and variants may be prepared by various methods known in the art for introducing appropriate nucleotide and amino acid changes. Two important variables in the construction of amino acid variants are the location of the mutation and the nature of the mutation. The amino acid variants of collagen are preferably constructed by mutating the polynucleotide to give an amino acid sequence that does not occur in nature. These amino acid alterations can be made at sites that differ in collagens from different species (variable positions) or in highly conserved regions (constant regions). Sites at such locations will typically be modified serially, e.g., by substituting first with conservative choices (e.g., hydrophobic amino acid to a different hydrophobic amino acid), and then with more distant choices (e.g., hydrophobic amino acid to a charged amino acid), and then deletions or insertions may be made at the target site.

Amino acids are divided into groups based on the properties of their side chains (polarity, charge, solubility, hydrophobicity, hydrophilicity, and/or the amphipatic nature): (1) hydrophobic (Leu, Met, Ala, Ile), (2) neutral hydrophobic (Cys, Ser, Thr), (3) acidic (Asp, Glu), (4) weakly basic (Asn, Gln, His), (5) strongly basic (Lys, Arg), (6) residues that influence chain orientation (Gly, Pro), and (7) aromatic (Trp, Tyr, Phe). Conservative changes encompass variants of an amino acid position that are within the same group as the “native” amino acid. Moderately conservative changes encompass variants of an amino acid position that are in a group that is closely related to the “native” amino acid (e.g., neutral hydrophobic to weakly basic). Non-conservative changes encompass variants of an amino acid position that are in a group that is distantly related to the “native” amino acid (e.g., hydrophobic to strongly basic or acidic).

Amino acid sequence deletions generally range from about 1 to 30 residues, preferably from about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells.

In another embodiment of the invention, a polynucleotide of the present invention may be ligated to a heterologous sequence to encode a fusion protein. For example, a fusion protein may be engineered to contain a cleavage site located between an α1(I) bovine collagen sequence of the present invention and the heterologous protein sequence, so that the α1(I) collagen may be cleaved away from the heterologous moiety.

Polynucleotide variants can also be generated according to methods well-known in the art. In one method of the present invention, polynucleotides are changed via site-directed mutagenesis. This method uses oligonucleotide sequences that encode the polynucleotide sequence of the desired amino acid variant, as well as a sufficient adjacent nucleotide on both sides of the changed amino acid to form a stable duplex on either side of the site of being changed. In general, the techniques of site-directed mutagenesis are well known to those of skill in the art and this technique is exemplified by publications such as, for example, Edelman et al. (1983) DNA 2:183. A versatile and efficient method for producing site-specific changes in a polynucleotide sequence is described in, e.g., by Zoller and Smith (1982) Nucleic Acids Res. 10:6487-6500.

As known in the art, nucleic acid mutations do not necessarily alter the amino acid sequence encoded by a polynucleotide sequence while providing unique restriction sites useful for manipulation of the molecule. Thus, the modified molecule can be made up of a number of discrete regions, or D-regions, flanked by unique restriction sites. These discrete regions of the molecule are herein referred to as cassettes. Molecules formed of multiple copies of a cassette are encompassed by the present invention. Recombinant or mutant nucleic acid molecules or cassettes, which provide desired characteristics, such as resistance to endogenous enzymes such as collagenase, are also encompassed by the present invention. (See, e.g., Maniatis et al., supra; and Ausubel et al., supra.)

It will be appreciated by those skilled in the art that, as a result of the degeneracy of the genetic code, a multitude of polynucleotide sequences encoding the polypeptides of the present invention, or functional equivalents thereof, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code.

The invention also encompasses production of polynucleotide sequences, or fragments thereof, encoding the polypeptides of the present invention or functional equivalents thereof, entirely by synthetic chemistry. After production, the synthetic sequence may be inserted into any of the many available expression vectors and cell systems using reagents that are well known in the art. Moreover, synthetic chemistry may be used to introduce mutations into a polynucleotide sequence encoding a collagen or functional equivalents thereof.

PCR may also be used to create variants of the present invention. When small amounts of template nucleic acid are used as starting material, primer(s) that differs slightly in sequence from the corresponding region in the template nucleic acid can generate the desired amino acid variant. PCR amplification results in a population of product polynucleotide fragments that differ from the polynucleotide template encoding the collagen at the position specified by the primer. The product fragments replace the corresponding region in the plasmid, creating the desired nucleic acid or amino acid variant.

Due to the inherent degeneracy of the genetic code, other polynucleotide sequences which encode substantially the same or functionally equivalent polypeptide sequences are encompassed by the present invention, and all degeneration variants and codon-optimized sequences are specifically contemplated. Encoding polynucleotide sequences that are natural, synthetic, semi-synthetic, or recombinant may be used in the practice of the claimed invention. Such polynucleotide sequences include those capable of hybridizing to the appropriate polynucleotide sequence under stringent conditions.

As naturally produced, collagens are structural proteins comprised of one or more collagen subunits which together form at least one triple-helical domain. A variety of enzymes are utilized in order to transform the collagen subunits into procollagen or other precursor molecules, and then into mature collagen. Such enzymes include, for example, prolyl-4-hydroxylase, C-proteinase, N-proteinase, lysyl oxidase, lysyl hydroxylase, etc.

Prolyl 4-hydroxylase is a α₂β₂ tetramer, and plays a central role in the biosynthesis of all collagens, 4-hydroxyproline residues stabilize the folding of the newly synthesized polypeptide chains into stable triple-helical molecules. (See, e.g., Prockop et al. (1995) Annu. Rev. Biochem. 64:403-434; Kivirikko et al. (1992) “Post-Translational Modifications of Proteins,” pp. 1-51; and Kivirikko et al. (1989) FASEB J. 3:1609-1617.) Additionally, the level of expression of type III collagen was lower in the absence of recombinant prolyl 4-hydroxylase than in its presence. Human isoforms of prolyl 4-hydroxylase have been cloned and characterized. (See, e.g., Helaakoski et al. (1995) Proc. Natl. Acad. Sci. 92:4427-4431; U.S. Patent No. 5,928,922.)

Lysyl hydroxylase, an α2 homodimer, catalyzes the post-translational modification of collagen to form hydroxylysine in collagens. See generally, Kivirikko et al. (1992) Post-Translational Modifications of Proteins, Harding, J. J., and Crabbe, M. J. C., eds., CRC Press, Boca Raton, Fla.; and Kivirikko (1995) Principles of Medical Biology, Vol. 3 Cellular Organelles and the Extracellular Matrix, Bittar, E. E., and Bittar, N., eds., JAI Press, Greenwich, Great Britain. Isoforms of lysyl hydroxylase have been cloned and identified. (See, e.g. Passoja et al. (1998) Proc. Natl. Acad. Sci. 95(18):10482-10486; and Valtavaara et al. (1997) J. Biol. Chem. 272(11):6831-6834.)

C-proteinase processes the assembled procollagen by cleaving off the C-terminal ends of the procollagens that assist in assembly of, but are not part of, the triple helix of the collagen molecule. (See, e.g., Kadler et al. (1987) J. Biol. Chem. 262:15969-15701; and Kadler et al. (1990) Ann. NY Acad. Sci. 580:214-224.)

N-proteinase processes the assembled procollagen by cleaving off the N-terminal ends of the procollagens that assist in the assembly of, but are not part of, the collagen triple helix. (See, e.g., Hojima et al. (1994)J. Biol. Chem. 269:11381-11390.)

Lysyl oxidase is an extracellular copper enzyme that catalyzes the oxidative deamination of the α-amino group in certain lysine and hydroxylysine residues to form a reactive aldehyde. These aldehydes then undergo an aldol condensation to form aldols, which cross links collagen fibrils. Information on the DNA and protein sequence of lysyl oxidase can found, for example, in Kivirikko (1995), supra; Kagan (1994) Path. Res. Pract. 190: 910-919; Kenyon et al. (1993) J. Biol. Chem. 268(25):18435-18437; Wu et al. (1992) J. Biol. Chem. 267(34):24199-24206; Manani et al. (1992) Matrix 12(3):242-248; and Hamalainen et al. (1991) Genomics 11(3):508-516.

The nucleic acid sequences encoding a number of these post-translational enzymes have been reported. (See, e.g., Vuori et al. (1992) Proc. Natl. Acad. Sci. USA 89:7467-7470; and Kessler et al. (1996) Science 271:360-362. The nucleic acid sequences encoding various post-translational enzymes may also be determined according to the methods generally described above and include use of appropriate probes and nucleic acid libraries.

The recombinant animal gelatins of the present invention may be derived from animal collagens using a variety of procedures known in the art. (See, e.g., Veis, A. (1965) International Review of Connective Tissue Research, 3:113-200.) For example, a common feature of current processes is the denaturation of the secondary structure of the collagen protein, and in the majority of instances, an alteration in either the primary or tertiary structure of the collagen. Thus, the animal collagens of the present invention can be processed using different procedures depending on the type of gelatin desired.

Recombinant animal gelatins of the present invention can be derived from recombinantly produced collagen or procollagens or other collagenous polypeptides by a variety of methods known in the art. For example, gelatin may be derived directly from cell mass or culture media by taking advantage of gelatin's solubility at elevated temperatures and its stability conditions of low or high pH, low or high salt concentration and high temperatures. Methods, processes, and techniques of producing gelatin compositions from collagen include denaturing the triple helical structure of the collagen utilizing detergents, heat, or various denaturing agents well known in the art. In addition, various steps involved in the extraction of gelatin from animal or slaughterhouse sources, including treatment with lime or acids, heat extraction in aqueous solution, ion exchange chromatography, cross-flow filtration and various methods of drying can be used to derive the gelatin of the present invention from recombinant collagen.

### Expression

The present methods of producing animal collagens and gelatins can be applied in a variety of recombinant systems available to those in the art. A number of these recombinant systems are described herein, although it is to be understood that application of the present methods is not to be limited to the systems illustrated for example below.

In order to express the recombinant animal collagens and gelatins of the present invention, or polypeptides from which the recombinant gelatins can be derived, the encoding polynucleotide is inserted into an appropriate expression vector, i.e., a vector which contains the necessary elements for the transcription and translation of the inserted coding sequence, or in the case of an RNA viral vector, the necessary elements for replication and translation.

Methods which are well known to those skilled in the art can be used to construct expression vectors containing the polynucleotides of the invention and appropriate transcriptional/translational control signals. These methods include standard DNA cloning techniques, e.g., in vitro recombinant techniques, synthetic techniques and in vivo recombination/genetic recombination. (See, for example, the techniques described in Maniatis et al., supra; and Ausubel et al., supra.)

The expression elements of different systems vary in their strength and specificities. Depending on the host/vector system utilized, any of a number of suitable transcription and translation elements, including constitutive and inducible promoters, may be used in the expression vector. For example, when cloning in bacterial systems, inducible promoters such as pL of bacteriophage γ plac, ptrp, ptac (ptrp-lac hybrid promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5 K promoter) may be used; when generating cell lines that contain multiple copies of a collagen DNA, SV40-, BPV- and EBV-based vectors may be used with an appropriate selectable marker.

Specific initiation signals may also be required for efficient translation of inserted sequences. These signals include the ATG initiation codon and adjacent sequences. In cases where the entire collagen gene, including its own initiation codon and adjacent sequences, is inserted into the appropriate expression vector, no additional translational control signals may be needed. However, in cases where only a portion of a collagen coding sequence is inserted, exogenous translational control signals, including the ATG initiation codon, must be provided. Furthermore, the initiation codon must be in phase with the reading frame of the collagen coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See, e.g., Bittner et al. (1987) Methods in Enzymol. 153:516-544).

The polypeptides of the invention may be expressed as secreted proteins. When the engineered cells used for expression of the proteins are non-human host cells, it is often advantageous to replace the secretory signal peptide of the collagen protein with an alternative secretory signal peptide which is more efficiently recognized by the host cell's secretory targeting machinery. The appropriate secretory signal sequence is particularly important in obtaining optimal fungal expression of mammalian genes. For example, see, e.g., Brake et al. (1984) Proc. Natl. Acad. Sci. USA 81:4642. Other signal sequences for prokaryotic, yeast, fungi, insect or mammalian cells are well known in the art, and one of ordinary skill could easily select a signal sequence appropriate for the host cell of choice.

The vectors of this invention may autonomously replicate in the host cell, or may integrate into the host chromosome. Suitable vectors with autonomously replicating sequences are well known for a variety of bacteria, yeast, and various viral replications sequences for both prokaryotes and eukaryotes. Vectors may integrate into the host cell genome when they have a nucleic acid sequence homologous to a sequence found in the genomic DNA of the host cell.

In one embodiment, the expression vectors of the present invention comprise a selectable marker, which encodes a product necessary for the host cell to grow and survive under certain conditions. Typical selection genes include genes encoding proteins that confer resistance to an antibiotic or other toxin (e.g., tetracycline, ampicillin, neomycin, methotrexate, etc.), proteins that complement an auxotrophic requirement of the host cell, etc. Other examples of selection genes include the herpes simplex virus thymidine kinase (Wigler et al. (1977) Cell 11:223), hypoxanthine-guanine phosphoribosyltransferase (Szybalska et al. (1962) Proc. Natl. Acad. Sci. USA 48:2026), and adenine phosphoribosyltransferase (Lowy et al. (1980) Cell 22:817) genes, which can be employed in tk⁻, hgprt⁻; or aprtf⁻ cells, respectively.

Antimetabolite resistance can be used as the basis of selection, such as with the use of dhfr which confers resistance to methotrexate; gpt, which confers resistance to mycophenolic acid; neo, which confers resistance to the aminoglycoside G-418; and hygro, which confers resistance to hygromycin. (See, e.g., Wigler et al. (1980) Proc. Natl. Acad. Sci. USA 77:3567; O'Hare et al. (1981) Proc. Natl. Acad. Sci. USA 78:1527; Mulligan et al. (1981) Proc. Natl. Acad. Sci. USA 78:2072; Colberre-Garapin et al. (1981) J. Mol. Biol. 150:1; and Santerre et al. (1984) Gene 30:147.) Additional selectable genes include trpB, which allows cells to utilize indole in place of tryptophan; hisD, which allows cells to utilize histinol in place of histidine; and odc (ornithine decarboxylase) which confers resistance to the omithine decarboxylase inhibitor, 2-(difluoromethyl)-DL-ornithine, DFMO. (See, e.g., Hartman et al. (1988) Proc. Natl. Acad. Sci. USA 85:8047 and McConlogue L., In: Current Communications in Molecular Biology, Cold Spring Harbor Laboratory, Ed. (1987)).

Elements necessary for the expression vectors of the invention include sequences for initiating transcription, e.g., promoters and enhancers. Promoters are untranslated sequences located upstream from the start codon of the structural gene that control the transcription of the nucleic acid under its control. Inducible promoters are promoters that alter their level of transcription initiation in response to a change in culture conditions, e.g., the presence or absence of a nutrient. One of skill in the art would know of a large number of promoters that would be recognized in host cells suitable for the present invention. These promoters are operably linked to the DNA encoding the collagen by removing the promoter from its native gene and placing the collagen encoding DNA 3′ of the promoter sequence.

Promoters useful in the present invention include, but are not limited to, the lactose promoter, the alkaline phosphatase promoter, the tryptophan promoter, hybrid promoters such as the tac promoter, promoter for 3-phosphoglycerate kinase, other glycolytic enzyme promoters (hexokinase, pyruvate decarboxylase, phophofructosekinase, glucose-6-phosphate isomerase, etc.), the promoter for alcohol dehydrogenase, the metallothionein promoter, the maltose promoter, the galactose promoter, promoters from the viruses polyoma, fowlpox, adenovirus, bovine papilloma virus, avian sarcoma virus, cytomegalovirus, retroviruses, SV40, and promoters from target eukaryotes including the glucoamylase promoter from Aspergillus, the actin promoter or an immunoglobin promoter from a mammal, and native collagen promoters. (See, e.g., de Boer et al. (1983) Proc. Natl. Acad. Sci. USA 80:21-25; Hitzeman et al. (1980) J. Biol. Chem. 255:2073; Fiers et al. (1978) Nature 273:113; Mulligan and Berg (1980) Science 209:1422-1427; Pavlakis et al. (1981) Proc. Natl. Acad. Sci. USA 78:7398-7402; Greenway et al. (1982) Gene 18:355-360; Gray et al. (1982) Nature 295:503-508; Reyes et al. (1982) Nature 297:598-601; Canaani and Berg (1982) Proc. Natl. Acad. Sci. USA 79:5166-5170; Gorman et al. (1982) Proc. Natl. Acad. Sci. USA 79:6777-6781; and Nunberg et al. (1984) Mol. and Cell. Biol. 11(4):2306-2315.)

Transcription of the coding sequence from the promoter is often increased by inserting an enhancer sequence in the vector. Enhancers are cis-acting elements, usually about from 10 to 300 bp, that act to increase the rate of transcription initiation at a promoter. Many enhancers are known for both eukaryotes and prokaryotes, and one of ordinary skill could select an appropriate enhancer for the host cell of interest. (See, e.g., Yaniv (1982) Nature 297:17-18.)

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cells lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include, but are not limited to, CHO, VERO, BHK, HeLa, COS, MDCK, 293, WI38, etc. Additionally, host cells may be engineered to express various enzymes to ensure the proper processing of the encoded polypeptide. For example, the gene for prolyl 4-hydroxylase may be co-expressed with a polynucleotide encoding a collagen or fragments or variants thereof to achieve proper hydroxylation.

For long-term, high-yield production of recombinant proteins, stable expression is preferred. For example, cell lines which stably express the collagens of the invention may be engineered. Rather than using expression vectors which contain viral origins of replication, host cells can be transformed with collagen encoding DNA controlled by appropriate expression control elements (e.g., promoter, enhancer, sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. Thus, the present methods may advantageously be used to engineer cell lines which express a desired animal collagen or fragments or variants thereof.

For example, expression of the present polypeptides driven by the galactose promoters can be induced by growing the culture on a non-repressing, non-inducing sugar so that very rapid induction follows addition of galactose; by growing the culture in glucose medium and then removing the glucose by centrifugation and washing the cells before resuspension in galactose medium; and by growing the cells in medium containing both glucose and galactose so that the glucose is preferentially metabolized before galactose-induction can occur.

The vectors expressing the polypeptides of the present invention, and the vectors expressing polynucleotides encoding any post-translational enzymes desired may be introduced into host cells to produce the encoded polypeptides, using techniques known to one of skill in the art. For example, host cells are transfected or infected or transformed with the above-described expression vectors, and cultured in nutrient media appropriate for selecting transductants or transformants containing the collagen encoding vector. Cell transfection can be carried out by a variety of methods available to those of skill in the art, such as, for example, by calcium phosphate precipitation, electroporation, and lipofection techniques. (See, e.g., Maniatis et al., supra, Ohta T. (1996) Nippon Rinsho 54(3):757-764; Trotter and Wood (1996) Mol Biotechnol 6(3):329-334; Mann and King (1989) J Gen Virol 70:3501-3505; and Hartig et al. (1991) Biotechniques 11(3):310.)

In one embodiment, the present invention provides a method in which more than one of the expression vectors encoding for the polypeptides of the present invention are inserted into cells, so that, e.g., trimeric collagens can be synthesized. For example, in one method of producing animal collagen according to the present invention, cells may be co-infected, co-transfected, or co-transformed with a first vector comprising a polynucleotide encoding a porcine α1(I) collagen, a second vector comprising a polynucleotide encoding a porcine α2(I) collagen, and third and fourth vectors comprising polynucleotides encoding the α subunit and the β subunit of prolyl 4-hydroxylase under conditions suitable for expression of the polypeptides and a fully hydroxylated, heterotrimeric porcine collagen.

In another method of the present invention, production of homotrimeric collagen is contemplated. For example, in the production of bovine collagen type III, cells may be co-infected, co-transfected, or co-transformed with a first vector comprising a polynucleotide encoding a bovine α1(III) collagen, a second vector comprising a polynucleotide encoding an α subunit of prolyl 4-hydroxylase, and a third vector comprising a polynucleotide encoding a β subunit of prolyl 4-hydroxylase. Other animal collagens, including mammalian collagens such as porcine, ovine, and equine collagens, and non-mammalian animal collagens, such as chicken and piscine collagen, may be produced using the same or similar co-expression methods and techniques, and variations thereof within the level of skill in the art.

Host cells containing coding sequence and expressing the biologically active gene product may be identified by any number of techniques known in the art. Such techniques include, for example, detecting the formation of nucleic acid hybridization complexes, detecting the presence or absence of marker gene functions assessing the level of transcription as measured by the expression of mRNA transcripts in the host cell, and detecting gene product as measured by immunoassay or by biological activity.

In the first approach, the presence of the present polynucleotide can be detected by, for example, detection of DNA-DNA or DNA-RNA hybridization complexes, or by amplification using probes comprising nucleotide sequences homologous to the animal collagen coding sequence, or portions, or derivatives thereof. Amplification-based assays involve the use of oligonucleotides or oligomers based on sequences homologous to the coding sequence of interest to detect transformants containing the encoding polynucleotides.

In the second approach, the recombinant expression vector/host system is identified and selected based upon the presence or absence of certain marker gene functions (e.g., thymidine kinase activity, resistance to antibiotics, resistance to methotrexate, transformation phenotype, occlusion body formation in baculovirus, etc.). For example, if the coding sequence is inserted within a marker gene sequence of the vector, recombinant cells containing coding sequence can be identified by the absence of the marker gene function. Alternatively, a marker gene can be placed in tandem with the coding sequence under the control of the same or different promoter used to control the expression of the coding sequence. Expression of the marker in response to induction or selection indicates expression of the coding sequence.

In the third approach, transcriptional activity of the coding region can be assessed by hybridization assays. For example, RNA can be isolated and analyzed by northern blot using a probe homologous to the coding sequence or particular portions thereof. Alternatively, total nucleic acids of the host cell may be extracted and assayed for hybridization to such probes.

In the fourth approach, the expression of a protein product can be assessed immunologically, for example by Western blots, immunoassays such as radioimmuno-precipitation, enzyme-linked immunoassays, and the like.

In one embodiment, the animal collagens of the present invention are secreted into the culture medium, and can be purified to homogeneity by various methods known in the art, for example, by chromatography. In one embodiment, recombinant animal collagens of the present invention are purified by size exclusion chromatography. However, other purification techniques known in the art can also be used, including ion exchange chromatography, and reverse-phase chromatography. (See, e.g., Maniatis et al., supra, Ausubel et al., supra, and Scopes (1994) Protein Purification: Principles and Practice, Springer-Verlag New York, Inc., NY.)

The present methods can be used in, although are not limited in application to, the expression systems listed below.

### Prokaryotic

In prokaryotic systems, such as bacterial systems, a number of expression vectors may be advantageously selected depending upon the use intended for the expressed polypeptide. For example, when large quantities of the animal collagens and gelatins of the invention are to be produced, such as for the generation of antibodies, vectors which direct the expression of high levels of fusion protein products that are readily purified may be desirable. Such vectors include, but are not limited to, the E. coli expression vector pUR278 (Rutheret al. (1983) EMBO J. 2:1791), in which the coding sequence may be ligated into the vector in frame with the lac Z coding region so that a hybrid AS-lac Z protein is produced; pIN vectors (Inouye et al. (1985) Nucleic Acids Res. 13:3101-3109 and Van Heeke et al. (1989) J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned polypeptide of interest can be released from the GST moiety.

### Yeast

In one embodiment, the present polypeptides are produced in a yeast expression system. In yeast, a number of vectors containing constitutive or inducible promoters known in the art may be used. (See, e.g., Ausubel et al., supra, Vol. 2, Chapter 13; Grant et al. (1987) Expression and Secretion Vectors for Yeast, in Methods in Enzymology, Ed. Wu & Grossman, Acad. Press, N.Y. 153:516-544; Glover (1986) DNA Cloning, Vol. II, IRL Press, Wash., D.C., Ch. 3; Bitter (1987) Heterologous Gene Expression in Yeast, in Methods in Enzymology, Eds. Berger & Kimmel, Acad. Press, N.Y. 152:673-684; and The Molecular Biology of the Yeast Saccharomyces, Eds. Strathem et al., Cold Spring Harbor Press, Vols. I and II (1982).)

Polypeptides of the present invention can be expressed using host cells, for example, from the yeast Saccharoinyces cerevisiae. This particular yeast can be used with any of a large number of expression vectors. Commonly employed expression vectors are shuttle vectors containing the 2μ origin of replication for propagation in yeast and the Col E1 origin for E. coli, for efficient transcription of the foreign gene. A typical example of such vectors based on 2μ plasmids is pWYG4, which has the 2μ ORI-STB elements, the GAL1-10 promoter, and the 2μ D gene terminator. In this vector, an NcoI cloning site is used to insert the gene for the polypeptide to be expressed, and to provide the ATG start codon. Another expression vector is pWYG7L, which has intact 2α ORI, STB, REP1 and REP2, and the GAL1-10 promoter, and uses the FLP terminator. In this vector, the encoding polynucleotide is inserted in the polylinker with its 5′ ends at a BamHI or Ncol site. The vector containing the inserted polynucleotide is transformed into S. cerevisiae either after removal of the cell wall to produce spheroplasts that take up DNA on treatment with calcium and polyethylene glycol or by treatment of intact cells with lithium ions.

Alternatively, DNA can be introduced by electroporation. Transformants can be selected, for example, using host yeast cells that are auxotrophic for leucine, tryptophane, uracil, or histidine together with selectable marker genes such as LEU2, TRP1, URA3, HIS3, or LEU2-D.

In one embodiment of the invention, the present polynucleotides are introduced into host cells from the yeast Pichia. Species of non-Saccharomyces yeast such as Pichia pastoris appear to have special advantages in producing high yields of recombinant protein in scaled up procedures. Additionally, a Pichia expression kit is available from Invitrogen Corporation (San Diego, Calif.).

There are a number of methanol responsive genes in methylotrophic yeasts such as Pichia pastoris, the expression of each being controlled by methanol responsive regulatory regions, also referred to as promoters. Any of such methanol responsive promoters are suitable for use in the practice of the present invention. Examples of specific regulatory regions include the AOX1 promoter, the AOX2 promoter, the dihydroxyacetone synthase (DAS), the P40 promoter, and the promoter for the catalase gene from P. pastoris, etc.

In other embodiments, the present invention contemplates the use of the methylotrophic yeast Hansenula polymorpha. Growth on methanol results in the induction of key enzymes of the methanol metabolism, such as MOX (methanol oxidase), DAS (dihydroxyacetone synthase), and FMHD (formate dehydrogenase). These enzymes can constitute up to 30-40% of the total cell protein. The genes encoding MOX, DAS, and FMDH production are controlled by strong promoters induced by growth on methanol and repressed by growth on glucose. Any or all three of these promoters may be used to obtain high-level expression of heterologous genes in H. polymorpha. Therefore, in one aspect of the invention, a polynucleotide encoding animal collagen or fragments or variants thereof is cloned into an expression vector under the control of an inducible H. polymorpha promoter. If secretion of the product is desired, a polynucleotide encoding a signal sequence for secretion in yeast is fused in frame with the polynucleotide. In a further embodiment, the expression vector preferably contains an auxotrophic marker gene, such as URA3 or LEU2, which may be used to complement the deficiency of an auxotrophic host.

The expression vector is then used to transform H. polymorpha host cells using techniques known to those of skill in the art. A useful feature of H. polytnorpha transformation is the spontaneous integration of up to 100 copies of the expression vector into the genome. In most cases, the integrated polynucleotide forms multimers exhibiting a head-to-tail arrangement. The integrated foreign polynucleotide has been shown to be mitotically stable in several recombinant strains, even under non-selective conditions. This phenomena of high copy integration further adds to the high productivity potential of the system.

### Fungi

Filamentous fungi may also be used to produce the present polypeptides. Vectors for expressing and/or secreting recombinant proteins in filamentous fungi are well known, and one of skill in the art could use these vectors to express the recombinant animal collagens of the present invention.

### Plant

In one aspect, the present invention contemplates the production of animal collagens and gelatins in plants and plant cells. In cases where plant expression vectors are used, the expression of sequences encoding the collagens of the invention may be driven by any of a number of promoters. For example, viral promoters such as the 35S RNA and 19S RNA promoters of CaMV (Brisson et al. (1984) Nature 310:511-514), or the coat protein promoter of TMV (Takamatsu et al. (1987) EMBO J. 6:307-311) may be used; alternatively, plant promoters such as the small subunit of RUBISCO (Coruzzi et al. (1984) EMBO J. 3:1671-1680; Broglie et al. (1984) Science 224:838-843) or heat shock promoters, e.g., soybean hsp17.5-E or hsp17.3-B (Gurley et al. (1986) Mol. Cell. Biol. 6:559-565) may be used. These constructs can be introduced into plant cells by a variety of methods known to those of skill in the art, such as by using Ti plasmids, Ri plasmids, plant virus vectors, direct DNA transformation, microinjection, electroporation, etc. For reviews of such techniques see, for example, Weissbach & Weissbach, Methods for Plant Molecular Biology, Academic Press, NY, Section VIII, pp. 421-463 (1988); Grierson & Corey, Plant Molecular Biology, 2d Ed., Blackie, London, Ch. 7-9 (1988); Transgenic Plants: A Production System for Industrial and Pharmaceutical Proteins, Owen and Pen eds., John Wiliey & Sons, 1996; Transgenic Plants, Galun and Breiman eds, Imperial College Press, 1997; and Applied Plant Biotechnology, Chopra, Malik, and Bhat eds., Science Publishers, Inc., 1999.

Plant cells do not naturally produce sufficient amounts of post-translational enzymes to efficiently produce stable collagen. Therefore, the present invention provides that, where hydroxylation is desired, plant cells used to express the present animal collagens are supplemented with the necessary post-translational enzymes to sufficiently produce stable collagen. In a preferred embodiment of the present invention, the post-translational enzyme is prolyl 4-hydroxylase.

Methods of producing the present animal collagens or gelatins in plant systems may be achieved by providing a biomass from plants or plant cells, wherein the plants or plant cells comprise at least one coding sequence is operably linked to a promoter to effect the expression of the polypeptide, and the polypeptide is then extracted from the biomass. Alternatively, the polypeptide can be non-extracted, i.e., expressed into the endosperm, etc.

Plant expression vectors and reporter genes are generally known in the art. (See, e.g., Gruber et al. (1993) in Methods of Plant Molecular Biology and Biotechnology, CRC Press.) Typically, the expression vector comprises a nucleic acid construct generated, for example, recombinantly or synthetically, and comprising a promoter that functions in a plant cell, wherein such promoter is operably linked to a nucleic acid sequence encoding an animal collagen or fragments or variants thereof, or a post-translational enzyme important to the biosynthesis of collagen.

Promoters drive the level of protein expression in plants. To produce a desired level of protein expression in plants, expression may be under the direction of a plant promoter. Promoters suitable for use in accordance with the present invention are generally available in the art. (See, e.g., PCT Publication No. WO 91/19806.) Examples of promoters that may be used in accordance with the present invention include non-constitutive promoters or constitutive promoters. These promoters include, but are not limited to, the promoter for the small subunit of ribulose-1,5-bis-phosphate carboxylase; promoters from tumor-inducing plasmids of Agrobacterium tumefaciens, such as the RUBISCO nopaline synthase (NOS) and octopine synthase promoters; bacterial T-DNA promoters such as mas and ocs promoters; and viral promoters such as the cauliflower mosaic virus (CaMV) 19S and 35S promoters or the figwort mosaic virus 35S promoter.

The polynucleotide sequences of the present invention may be under the transcriptional control of a constitutive promoter, directing expression of the collagen or post-translational enzyme in most tissues of a plant. In one embodiment, the polynucleotide sequence is under the control of the cauliflower mosaic virus (CaMV) 35S promoter. The double-stranded caulimorvirus family has provided the single most important promoter expression for transgene expression in plants, in particular, the 35S promoter. (See, e.g., Kay et al. (1987) Science 236:1299.) Additional promoters from this family such as the figwort mosaic virus promoter, etc., have been described in the art, and may also be used in accordance with the present invention. (See, e.g., Sanger et al. (1990) Plant Mol. Biol. 14:433-443; Medberry et al. (1992) Plant Cell 4:195-192; and Yin and Beachy (1995) Plant J. 7:969-980.)

The promoters used in the polynucleotide constructs of the present invention may be modified, if desired, to affect their control characteristics. For example, the CaMV promoter may be ligated to the portion of the RUBISCO gene that represses the expression of RUBISCO in the absence of light, to create a promoter which is active in leaves, but not in roots. The resulting chimeric promoter may be used as described herein.

Constitutive plant promoters having general expression properties known in the art may be used with the expression vectors of the present invention. These promoters are abundantly expressed in most plant tissues and include, for example, the actin promoter and the ubiquitin promoter. (See, e.g., McElroy et al. (1990) Plant Cell 2:163-171; and Christensen et al. (1992) Plant Mol. Biol. 18:675-689.)

Alternatively, the polypeptide of the present invention may be expressed in a specific tissue, cell type, or under more precise environmental conditions or developmental control. Promoters directing expression in these instances are known as inducible promoters. In the case where a tissue-specific promoter is used, protein expression is particularly high in the tissue from which extraction of the protein is desired. Depending on the desired tissue, expression may be targeted to the endosperm, aleurone layer, embryo (or its parts as scutellum and cotyledons), pericarp, stem, leaves tubers, roots, etc. Examples of known tissue-specific promoters include the tuber-directed class I patatin promoter, the promoters associated with potato tuber ADPGPP genes, the soybean promoter of β-conglycinin (7S protein) which drives seed-directed transcription, and seed-directed promoters from the zein genes of maize endosperm. (See, e.g., Bevan et al. (1986) Nucleic Acids Res. 14: 4625-38; Muller et al. (1990) Mol. Gen. Genet. 224:136-46; Bray (1987) Planta 172:364-370; and Pedersen et al. (1982) Cell 29:1015-26.)

In a preferred embodiment, the present polypeptides are produced in seed by way of seed-based production techniques using, for example, canola, corn, soybeans, rice and barley seed. In such a process, for example, the product is recovered during seed germination. (See, e.g., PCT Publication Numbers WO 9940210; WO 9916890; WO 9907206; U.S. Pat. No. 5,866,121; U.S. Pat. No. 5,792,933; and all references cited therein.)

Promoters that may be used to direct the expression of the polypeptides may be heterologous or non-heterologous. These promoters can also be used to drive expression of antisense nucleic acids to reduce, increase, or alter concentration and composition of the present animal collagens in a desired tissue.

Other modifications that may be made to increase and/or maximize transcription of the present polypeptides in a plant or plant cell are standard and known to those in the art. For example a vector comprising a polynucleotide sequence encoding a recombinant animal collagen or gelatin, or a polypeptide from which the recombinant animal gelatin may be derived, or a fragment or variant thereof, operably linked to a promoter may further comprise at least one factor that modifies the transcription rate of collagen or related post-translational enzymes, including, but not limited to, peptide export signal sequence, codon usage, introns, polyadneylation, and transcription termination sites. Methods of modifying constructs to increase expression levels in plants are generally known in the art. (See, e.g. Rogers et al. (1985) J. Biol. Chem. 260:3731; and Cornejo et al. (1993) Plant Mol Biol 23:567-58.) In engineering a plant system that affects the rate of transcription of the present collagens and related post-translational enzymes, various factors known in the art, including regulatory sequences such as positively or negatively acting sequences, enhancers and silencers, as well as chromatin structure can affect the rate of transcription in plants. The present invention provides that at least one of these factors may be utilized in expressing the recombinant animal collagens and gelatins described herein.

The vectors comprising the present polynucleotides will typically comprise a marker gene which confers a selectable phenotype on plant cells. Usually, the selectable marker gene will encode antibiotic resistance, with suitable genes including at least one set of genes coding for resistance to the antibiotic spectinomycin, the streptomycin phophotransferase (SPT) gene coding for streptomycin resistance, the neomycin phophotransferase (NPTH) gene encoding kanamycin or geneticin resistance, the hygromycin resistance, genes coding for resistance to herbicides which act to inhibit the action of acetolactate synthase (ALS), in particular, the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phophinothricin or basta (e.g. the bar gene), or other similar genes known in the art. The bar gene encodes resistance to the herbicide basta, the nptII gene encodes resistance to the antibiotics kanamycin and geneticin, and the ALS gene encodes resistance to the herbicide chlorsulfuron.

Typical vectors useful for expression of foreign genes in plants are well known in the art, including, but not limited to, vectors derived from the tumor-inducing (Ti) plasmid of Agrobacterium tumefaciens. These vectors are plant integrating vectors, that upon transformation, integrate a portion of the DNA into the genome of the host plant. (See, e.g., Rogers et al. (1987) Meth. In Enzymol. 153:253-277; Schardl et al. (1987) Gene 61:1-11; and Berger et al., Proc. Natl. Acad. Sci. U.S.A. 86:8402-8406.)

Vectors comprising sequences encoding the present polypeptides and vectors comprising post-translational enzymes or subunits thereof may be co-introduced into the desired plant. Procedures for transforming plant cells are available in the art, for example, direct gene transfer, in vitro protoplast transformation, plant virus-mediated transformation, liposome-mediated transformation, microinjection, electroporation, Agrobacterium mediated transformation, and particle bombardment. (See, e.g., Paszkowski et al. (1984) EMBO J. 3:2717-2722; U.S. Pat. No. 4,684,611; European Application No. 0 67 553; U.S. Pat. No. 4,407,956; U.S. Pat. No. 4,536,475; Crossway et al. (1986) Biotechniques 4:320-334; Riggs et al. (1986) Proc. Natl. Acad. Sci USA 83:5602-5606; Hinchee et al. (1988) Biotechnology 6:915-921; and U.S. Pat. No. 4,945,050.) Standard methods for the transformation of, e.g., rice, wheat, corn, sorghum, and barley are described in the art. (See, e.g., Christou et al. (1992) Trends in Biotechnology 10: 239 and Lee et al. (1991) Proc. Nat'l Acad. Sci. USA 88:6389.) Wheat can be transformed by techniques similar to those employed for transforming corn or rice. Furthermore, Casas et al. (1993) Proc. Nat'l Acad. Sci. USA 90:11212, describe a method for transforming sorghum, while Wan et al. (1994) Plant Physiol. 104: 37, teach a method for transforming barley. Suitable methods for corn transformation are provided by Fromm et al. (1990) Bio/Technology 8:833 and by Gordon-Kamm et al., supra.

Additional methods that may be used to generate plants that produce animal collagens of the present invention are well established in the art. (See, e.g., U.S. Pat. No. 5,959,091; U.S. Pat. No. 5,859,347; U.S. Pat. No. 5,763,241; U.S. Pat. No. 5,659,122; U.S. Pat. No. 5,593,874; U.S. Pat. No. 5,495,071; U.S. Pat. No. 5,424,412; U.S. Pat. No. 5,362,865; U.S. Pat. No. 5,229,112; U.S. Pat. No. 5,981,841; U.S. Pat. No. 5,959,179; U.S. Pat. No. 5,932,439; U.S. Pat. No. 5,869,720; U.S. Pat. No. 5,804,425; U.S. Pat. No. 5,763,245; U.S. Pat. No. 5,716,837; U.S. Pat. No. 5,689,052; U.S. Pat. No. 5,633,435; U.S. Pat. No. 5,631,152; U.S. Pat. No. 5,627,061; U.S. Pat. No. 5,602,321; U.S. Pat. No. 5,589,612; U.S. Pat. No. 5,510,253; U.S. Pat. No. 5,503,999; U.S. Pat. No. 5,378,619; U.S. Pat. No. 5,349,124; U.S. Pat. No. 5,304,730; U.S. Pat. No. 5,185,253; U.S. Pat. No. 4,970,168; European Publication No. EPA 00709462; European Publication No. EPA 00578627; European Publication No. EPA 00531273; European Publication No. EPA 00426641; PCT Publication No. WO 99/31248; PCT Publication No. WO 98/58069; PCT Publication No. WO 98/45457; PCT Publication No. WO 98/31812; PCT Publication No. WO 98/08962; PCT Publication No. WO 97/48814; PCT Publication No. WO 97/30582; and PCT Publication No. WO 9717459.)

### Insect

Another alternative expression system used in accordance with the present methods is an insect system. Baculoviruses are very efficient expression vectors for the large scale production of various recombinant proteins in insect cells. The methods as described in, for example, Luckow et al. (1989) Virology 170:31-39 and Gruenwald, S. and Heitz, J. (1993) Baculovirus Expression Vector System: Procedures & Methods Manual, Pharmingen, San Diego, Calif., can be employed to construct expression vectors containing a collagen coding sequence for the collagens of the invention and the appropriate transcriptional/translational control signals. For example, recombinant production of proteins can be achieved in insect cells, by infection of baculovirus vectors encoding the polypeptide. In one aspcect of the present invention, production of recombinant polypeptides with stable triple helices can involve the co-infection of insect cells with three baculoviruses, one encoding the animal collagen to be expressed and one each encoding the α subunit and β subunit of prolyl 4-hydroxylase. This insect cell system allows for production of recombinant proteins in large quantities. In one such system, Autographa californica nuclear polyhidrosis virus (AcNPV) is used as a vector to express foreign genes. The virus grows in Spodoptera frugiperda cells. Coding sequence for the polypeptides of the invention may be cloned into non-essential regions (for example the polyhedron gene) of the virus and placed under control of an AcNPV promoter (for example, the polyhedron promoter). Successful insertion of a coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed. (See, e.g., Smith et al. (1983) J. Virol. 46:584; and U.S. Pat. No.4,215,051). Further examples of this expression system may be found in, for example, Ausubel et al., supra.

### Animal

In animal host cells, a number of expression systems may be utilized. In cases where an adenovirus is used as an expression vector, polynucleotide sequences of the present invention may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the encoded polypeptides in infected hosts. (See, e.g., Logan & Shenk, Proc. Natl. Acad. Sci. USA 81:3655-3659 (1984)). Alternatively, the vaccinia 7.5 K promoter may be used. (See, e.g., Mackett et al. (1982) Proc. Natl. Acad. Sci. USA 79:7415-7419; Mackett et al. (1982) J. Virol. 49:857-864; and Panicali et al. (1982) Proc. Natl. Acad. Sci. USA 79:4927-4931.

A preferred expression system in mammalian host cells is the Semliki Forest virus. Infection of mammalian host cells, for example, baby hamster kidney (BHK) cells and Chinese hamster ovary (CHO) cells can yield very high recombinant expression levels. Semliki Forest virus is a preferred expression system as the virus has a broad host range such that infection of mammalian cell lines will be possible. More specifically, it is expected that the use of the Semliki Forest virus can be used in a wide range of hosts, as the system is not based on chromosomal intergration, and therefore will be a quick way of obtaining modifications of the recombinant animal collagens in studies aiming at identifying structure-function relationships and testing the effects of various hybrid molecules. Methods for constructing Semliki Forest virus vectors for expression of exogenous proteins in mammalian host cells are described in, for example, Olkkonen et al. (1994) Methods Cell Biol 43:43-53.

Transgenic animals may also be used to express the polypeptides of the present invention. Such systems can be constructed by operably linking the polynucleotide of the invention to a promoter, along with other required or optional regulatory sequences capable of effecting expression in mammary glands. Likewise, required or optional post-translational enzymes may be produced simultaneously in the target cells employing suitable expression systems. Methods of using transgenic animals to recombinantly produce proteins are known in the art. (See, e.g., U.S. Pat. No. 4,736,866; U.S. Pat. No. 5,824,838; U.S. Pat. No. 5,487,992; and U.S. Pat. No. 5,614,396.)

### Uses of Collagens and Gelatins

The recombinant collagens and gelatins of the present invention are useful in a variety of applications. Collagen is widely used in numerous applications in the medical, pharmaceutical, food, and cosmetic industries. For example, collagen is an important component of arterial sealants, bone grafts, drug delivery systems, dermal implants, hemostats, and incontinence implants. In treatments for autoimmune disorders such as rheumatoid arthritis, collagen has been evaluated in trials for its potential to induce oral-tolerance. Collagen is also applied in food products such as sausage casings, and other collagen-based casings derived from, for example, porcine, bovine, and ovine sources. In health and beauty applications, collagen can be found, for example, in cosmetics or facial and skin products such as moisturizers. To date, various collagens used in various applications are derived from animal sources using enzymatic and chemical processes. For example, commercially available bovine collagen is isolated from bovine tissues and bones, and is comprised of a mixture of primarily types I and III collagen. This form of collagen is also used as an injectable device in humans.

Gelatin appears in the manufacture or as a component of various pharmaceutical and medical products and devices, including pharmaceutical stabilizers, e.g., drug and vaccine, plasma extenders, sponges, hard and soft gelatin capsules, suppositories, etc. Gelatin's film-forming capabilities are employed in various film coating systems designed specifically for pharmaceutical oral solid dosage forms, including controlled release capsules and tablets.

Gelatin in various edible forms has long been used in the food and beverage industries. Gelatin serves as an emulsifier and thickener in various whipped toppings, as well as in soups and sauces. Gelatin is used as a flocculating agent in clarifying and fining various beverages, including wines and fruit juices. Gelatin is used in various low and reduced fat products as a thickener and stabilizer, and appears elsewhere as a fat substitute. Gelatin is also widely used in micro-encapsulation of flavorings, colors, and vitamins. Gelatin can also be used as a protein supplement in various high energy and nutritional beverages and foods, such as those prevalent in the weight-loss and athletic industries. As a film-former, gelatin is used in coating fruits, meats, deli items, and in various confectionery products, including candies and gum, etc.

In the cosmetics industry, gelatin appears in a variety of hair care and skin care products. Gelatin is used as a thickener and bodying agent in a number of shampoos, mousses, creams, lotions, face masks, lipsticks, manicuring solutions and products, and other cosmetic devices and applications. Gelatin is also used in the cosmetics industry in micro-encapsulation and packaging of various products.

Gelatin is used in a wide range of industrial applications. For example, gelatin is widely used as a glue and adhesive in various manufacturing processes. Gelatin can be used in various adhesive and gluing formulations, such as in the manufacture of remoistenable gummed paper packaging tapes, wood gluing, paper bonding of various grades of box boards and papers, and in various applications which provide adhesive surfaces which can be reactivated by remoistening.

Gelatin serves as a light-sensitive coating in various electronic devices and is used as a photoresist base in various photolithographic processes, for example, in color television and video camera manufacturing. In semiconductor manufacturing, gelatin is used in constructing lead frames and in the coating of various semiconductor elements. Gelatin is used in various printing processes and in the manufacturing of special quality papers, such as that used in bond and stock certificates, etc.

Gelatin is used in a variety of photographic applications, e.g., as a carrier for various active components in photographic solutions, including solutions used in X-ray and photographic film development. Gelatin, long used in various photoengraving techniques, is also included as a component of various types of film, and is heavily used in silver halide chemistry in various layers of film and paper products. Silver gelatin film appears in the form of microfiche film and in other forms of information storage. Gelatin is used as a self-sealing element of various films, etc.

Gelatin has also been a valuable substance for use in various laboratory applications. For example, gelatin can be used in various cell culture applications, providing a suitable surface for cell attachment and growth, e.g., plate or flask coating, or providing a surface for cell attachment and growth. Hydrolyzed or low gel strength gelatin is used as a biological buffer in various processes, for example, in coating and blocking solutions used in assays such as enzyme-linked immunosorbent assays (ELISAs) and other immunoassays. Gelatin is also a component in various gels used for biochemical and electrophoretic analysis, including enzymography gels.

### EXAMPLES

The following examples are provided solely to illustrate the claimed invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

### Example 1

### Sequencing of Bovine Procollagen Type I α1

Experiments were performed to generate α1(I) collagen gene fragments by PCR from a commercial bovine aorta smooth muscle cDNA library (Stratagene #936705) that had been a successful source of bovine collagen (I) alpha 2 gene fragments in initial PCR experiments. In this initial screening process, PCR primers were designed from the bovine mRNA sequence (Shirai et al. (1998) Matrix Biology 17:85-88) of collagen (I) α2, and PCR amplifications performed, and DNA fragments were obtained. Although the commercial library was shown to contain the complete coding region of the bovine collagen (I) alpha 2 gene, attempts to generate fragments of the bovine α1(I) collagen gene using a variety of human α1(I) collagen sequence PCR primers proved unsuccessful. An alternative source of a cDNA pool likely to contain a bovine α1(I) collagen transcript was sought.

An ATCC bovine skin cell line (CRL-6054; skin, normal, bovine) was grown to approximately 60% confluency and total RNA was isolated (Qiagen RNeasy). A cDNA pool was prepared from the resulting RNA by RT-PCR (Clontech RT-for-PCR reagents). This cDNA pool was used as the template source for subsequent PCR experiments of overlapping gene fragments.

Primers were designed from known human α1(I) collagen mRNA sequence, and used to amplify overlapping segments of the open reading frame (ORF) of the gene. (Mackay et al. (1993) Human Molecular Genetics 2(8):1155-1160). The PCR primers were engineered to amplify fragments located in the triple helical coding region of the human α1(I) collagen gene and are set forth in Table 1.

TABLE 1

| | | |
|---|---|---|
| SEQ ID NO: | PRIMER | SEQUENCE |

| | | |
|---|---|---|
| 13 | SSCP 1F | CCGGCTCCTGCTCCTCTTAG |
| 14 | SSCP 1REV | GCCAGGAGCACCAGCAATAC |
| 15 | SSCP 2F | GCTGATGGACAGCCTGGTGC |
| 16 | SSCP 2REV | GCCCTGGAAGACCAGCTGCA |
| 17 | SSCP 3F | CCTGGCCTTAAGGGAATGCC |
| 18 | SSCP 3REV | GCGCCAGGAGAACCGTCTCG |
| 19 | SSCP 4F | CCGAAGGTTCCCCTGGACGA |
| 20 | SSCP 4REV | CGGTCATGCTCTCGCCGAAC |

The primers were used to obtain four overlapping bovine PCR fragments covering the triple helical portion of the bovine α1(I) collagen gene. PCR (Clontech, Advantage GC-Rich cDNA PCR kit; all PCR primers used @ 100 pmol each per reaction) was performed using a thermal cycler (Hybaid, non-refrigerated) under the following conditions:

| | |
|---|---|
| Step 1: | 94° C. for 4 minutes |
| Step 2: | 28 cycles of: |
| | 68° C. for 3 minutes |
| | 94° C. for 30 seconds |
| | 60° C. for 30 seconds |
| Step 3: | 68° C. for 10 minutes |
| | 30° C. for 1 second |
| | Hold @ room temperature |

All PCR products were initially screened by gel electrophoresis, and those of the predicted size were purified by agarose gel electrophoresis and/or column purification (Qiagen Qiaquick). To facilitate sequencing the selected PCR fragments were cloned into a vector (pCRII-TOPO kit, Invitrogen). Multiple clones of each PCR fragment were sequenced with an external vector sequencing primers (M13 forward and reverse) using an ABI 373 automated sequencer (ABI PRISM® BigDye™ Terminator Cycle Sequencing Kit, Perkin-Elmer). Sequence data obtained was analyzed with the use of SEQMAN software (DNASTAR) and a consensus sequence determined for the cloned fragments.

The resulting bovine α1(I) collagen sequence obtained was used to design internal bovine collagen sequencing primers, which were then used to complete the sequencing of these bovine clones. These primers were designed with the aid of primer design software (RightPrimer, BioDisk), and are set forth in Table 2.

TABLE 2

| | | |
|---|---|---|
| SEQ | | |
| ID NO: | PRIMER | SEQUENCE |

| | | |
|---|---|---|
| 21 | B C1A1 SP 502F | CCCCAGTTGTCTTACGGCTATG |
| 22 | B C1A1 SP 502REV | CATAGCCGTAAGACAACTGGGG |
| 23 | B C1A1 SP 886F | GGTAGCCCCGGTGAAAATG |
| 24 | B C1A1 SP 886REV | CATTTTCACCGGGGCTACC |
| 25 | B C1A1 SP 1302F | GCCCCAAGGGTAACAGCGGT |
| 26 | B C1A1 SP 1302REV | ACCGCTGTTACCCTTGGGGC |
| 27 | B C1A1 SP 1560F | TCCTGGCCCTGCTGGCCCCAAA |
| 28 | B C1A1 SP 1560REV | TTTGGGGCCAGCAGGGCCAGGA |
| 29 | B C1A1 SP 1770F | TGGACCTAAAGGTGCTGCTGGA |
| 30 | B C1A1 SP 1770REV | TCCAGCAGCACCTTTAGGTCCA |
| 31 | B C1A1 SP 1997F | GAACAGGGTGITCCTGGAGA |
| 32 | B C1A1 SP 1997REV | TCTCCAGGAACACCCTGTTC |
| 33 | B C1A1 SP 2289F | GGCAAAGATGGCGTCCGT |
| 34 | B C1A1 SP 2289REV | ACGGACGCCATCTTTGCC |
| 35 | B C1A1 SP 2592F | GCTAAAGGCGAACCTGGCGA |
| 36 | B C1A1 SP 2592REV | TCGCCAGGTTCGCCTTTAGC |
| 37 | B C1A1 SP 3198F | GCCGGCAAGAGCGGTGATCGT |
| 38 | B C1A1 SP 3198REV | ACGATCACCGCTCTTGCCGGC |
| 39 | B C1A1 SP 3648F | CGATGGTGGCCGCTACTAC |
| 40 | B C1A1 SP 3648REV | GTAGTAGCGGCCACCATCG |
| 41 | B C1A1 SP 4007F | AGAGCATGACCGAAGGGCGAATT |
| 42 | B C1A1 SP 4007REV | AATTCGCCCTTCGGTCATGCTCT |

After producing bovine PCR products with the eight SSCP human primers shown in Table 1 (SEQ ID NOs: 13 through 14), three additional PCR fragments were amplified, overlapping the initial bovine clones, and extending to the putative ends (by analogy with the human α1(I) collagen sequence) of the ORF. The PCR primers used for this amplification are set forth in Table 3.

TABLE 3

| | | |
|---|---|---|
| SEQ ID NO: | PRIMER | SEQUENCE |

| | | |
|---|---|---|
| 43 | H AVR II F | TTAATTCCTAGGATGTTCAGCTTTGTGGACCTCCGGCTC |
| 44 | H EAR 1 F | TGCCACTCTGACTGGAAGAGTGGAGAGTACTG |
| 45 | H NOT1 REV | TTTTCCTTTTGCGGCCGCTTACAGGAAGCAGACAGGGCCAACGTC |

The resulting fragments were cloned and sequenced, and a consensus sequence was established for most of the ORF of the gene by pairing of the following primers: H AVR II (SEQ ID NO: 43) with SSCP 1REV (SEQ ID NO: 14); H EAR 1 F (SEQ ID NO: 44) with H NOT1 REV (SEQ ID NO: 45); and SSCP 4F (SEQ ID NO: 19) with H NOT1 REV (SEQ ID NO: 45).

To obtain the 5′ and 3′ ends of the cDNA clone, nested PCR primers were designed from the bovine sequence by RACE (rapid amplification of cDNA ends) methodology (SMART RACE cDNA Amplification Kit, Clontech), and with the aid of primer design software. For increased specificity, the primers were designed to have particularly high melting temperatures. The designed primers are set forth in Table 4.

TABLE 4

| | | |
|---|---|---|
| SEQ | | |
| ID NO: | PRIMER | SEQUENCE |

| | | |
|---|---|---|
| 46 | GS BC1A1 | GTCATGGTACCTGAGGCCGTTCTGTACGCA |
| | 118REV | |
| 47 | GS BC1A1 | ACGTCATCGCACAGCACGTTGCCGTTGTC |
| | 190REV | |
| 48 | GS BC1A1 | AGGACAGTCCTTAAGTTCGTCGCAGATCACGTCA |
| | 213REV | |
| 49 | GS BC1A1 | AGGGAGGCCAGCTGTTCCAGGCAATC |
| | 761REV | |
| 50 | GS BC1A1 | CCGAAGGTTCCCCTGGACGAGATGGTT |
| | 3085F | |
| 51 | GS BC1A1 | CGTGGTGACAAGGGTGAGACAGGCGAACA |
| | 3305F | |
| 52 | GS BC1A1 | CGGGCTGATGATGCCAATGTGGTCCGT |
| | 3675F | |
| 53 | GS BC1A1 | AACATGGAAACCGGTGAGACCTGTGTATACCC |
| | 3905F | |

The total bovine mRNA described above was further utilized to prepare new cDNA pools with the necessary external priming sites for use as PCR templates. PCR products were obtained at both the 5′ and 3′ ends of the gene using: (1) touchdown PCR techniques; (2) the newly designed bovine RACE PCR primers; and (3) materials supplied in the kit. Two touchdown PCR programs were used in a Peltier-cooled thermal cycler using the following protocol and conditions:

72° C.-68° C. touchdown program I:

Step 1: 8 cycles with the following conditions:

94° C. for 10 seconds

72° C. for 10 seconds, each cycle thereafter drop 0.5° C.

72° C. for 3 minutes

Step 2: 28 cycles of the following conditions:

94° C. for 10 seconds

68° C. for 10 seconds

72° C. for 3 minutes

72° C. for 10 minutes

4° C. HOLD

68° C.-64°0 C. touchdown program II:

Step 1: 8 cycles of the following conditions:

94° C. for 10 seconds

68° C. for 10 seconds, each cycle thereafter drop 0.5° C.

72° C. for 3 minutes

Step 2: 28 cycles of the following conditions:

94° C. for 10 seconds

64° C. for 10 seconds

72° C. for 3 minutes

72° C. for 10 minutes

4° C. HOLD

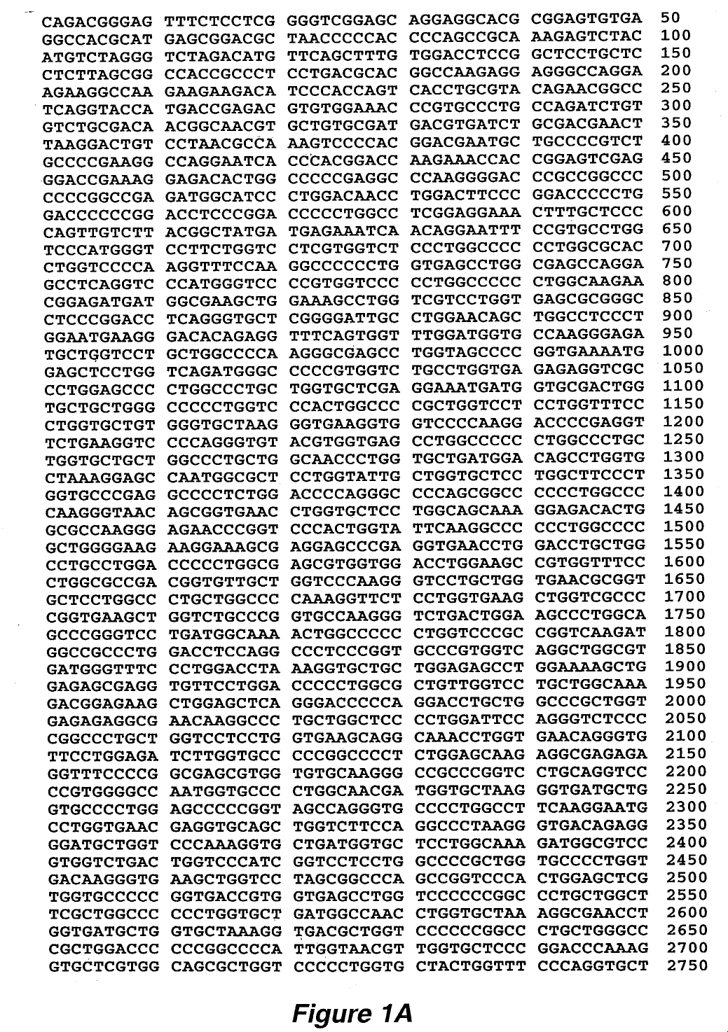

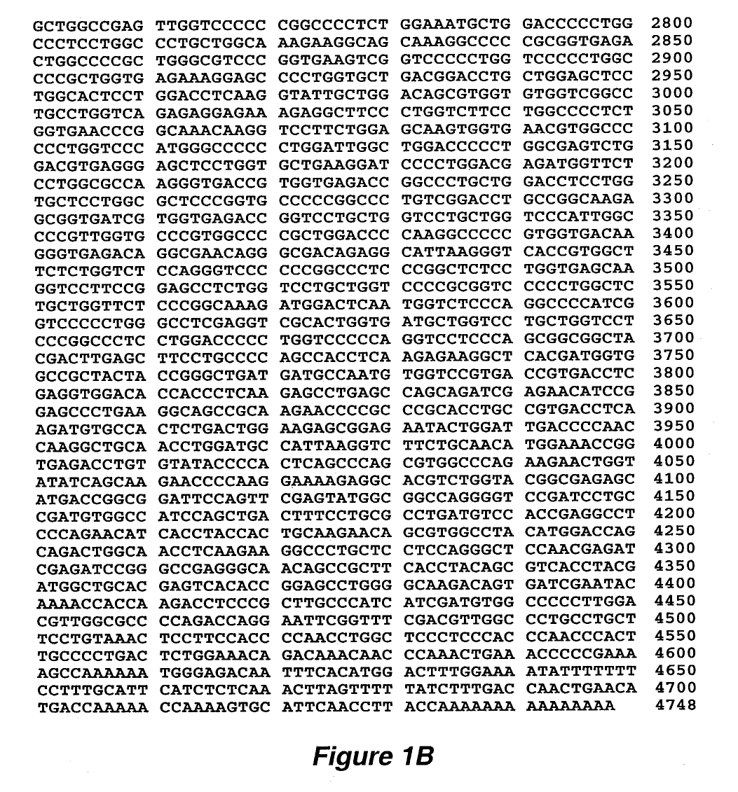

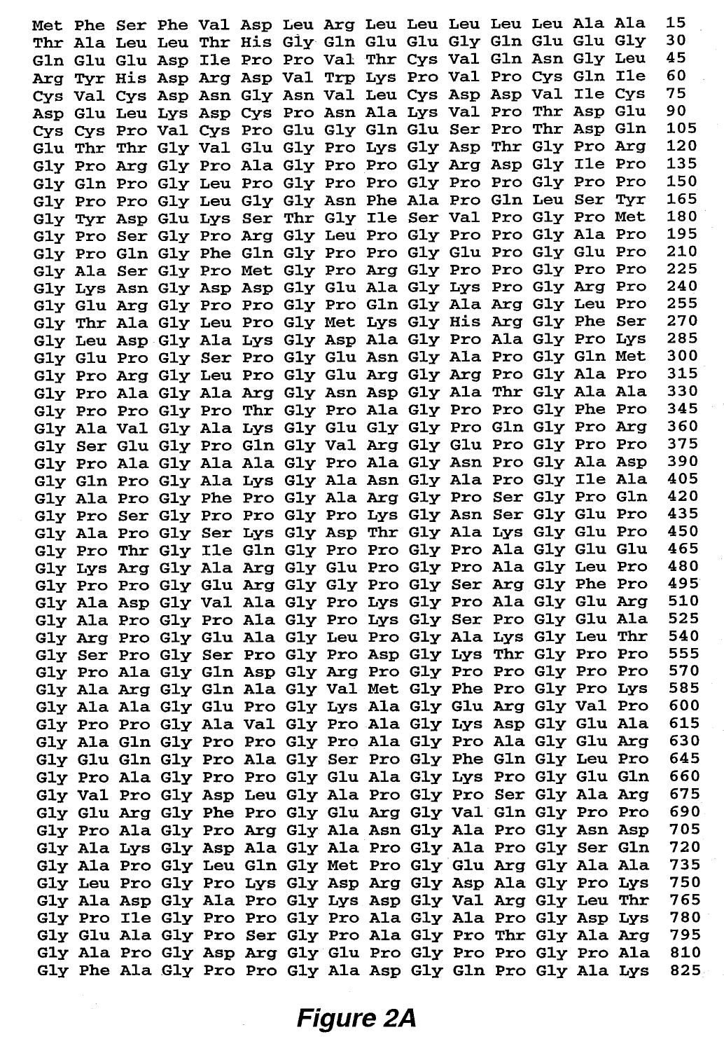

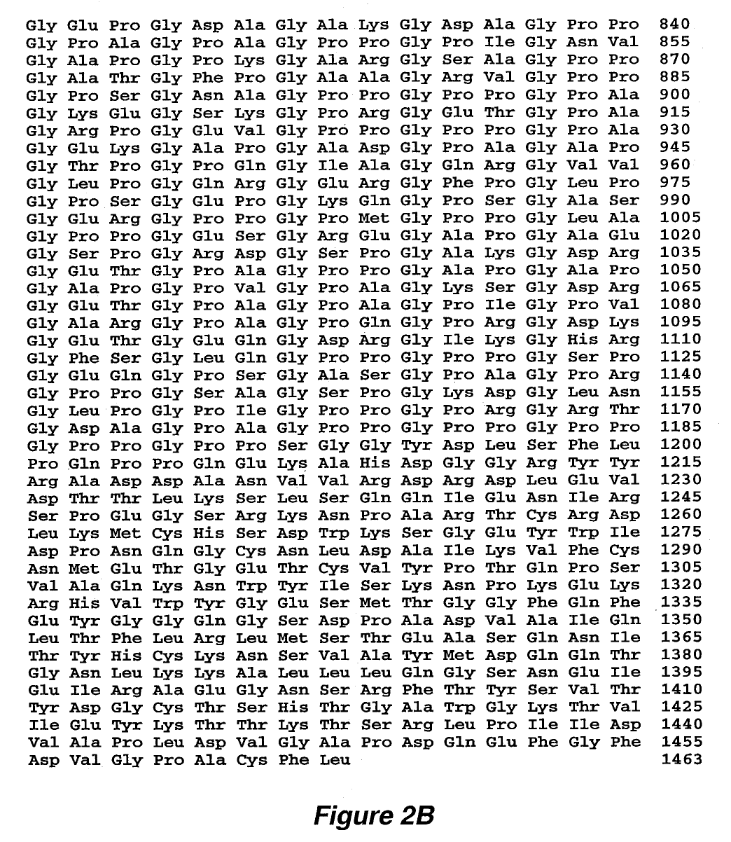

The resulting fragments were examined by 1.2% agarose gel electrophoresis, and subsequent cloning and sequencing analysis was performed. PCR products resulting from both programs were used. The resulting sequences overlapped the previously cloned bovine α1(I) collagen sequences, and encoded the 5′ and 3′ ends of the ORF as well as the contiguous untranslated cDNA regions. The nucleotide sequence for bovine procollagen type Iα1 is shown in FIGS. 1A through IC (SEQ ID NO: 1). The corresponding amino acid sequence is described in FIGS. 2A through 2D (SEQ ID NO: 2).

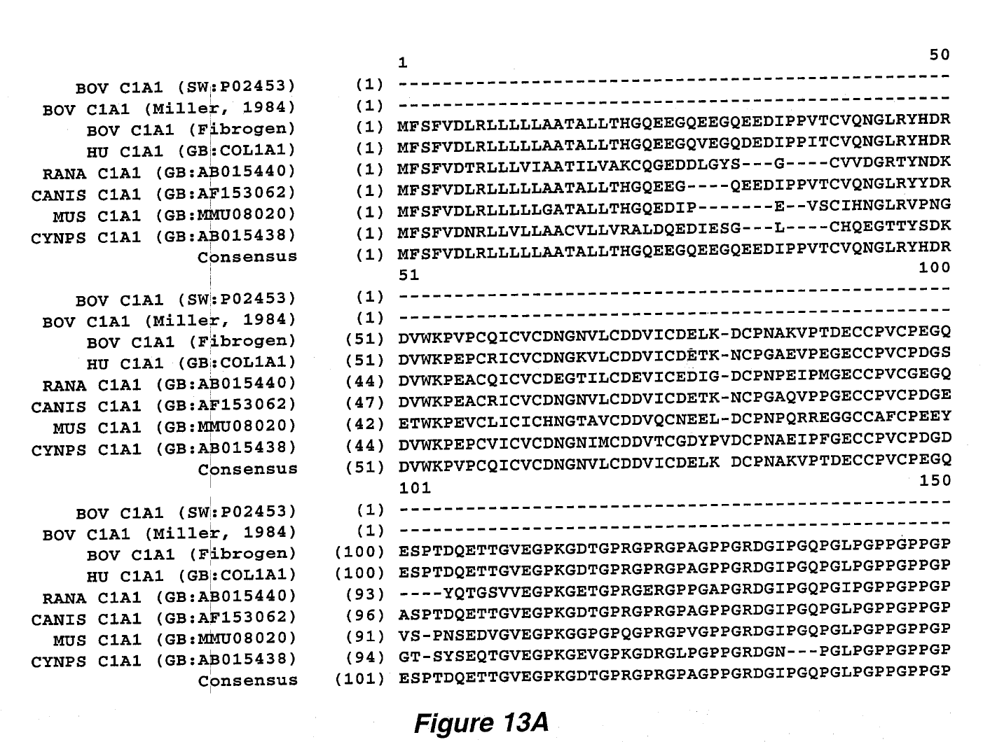

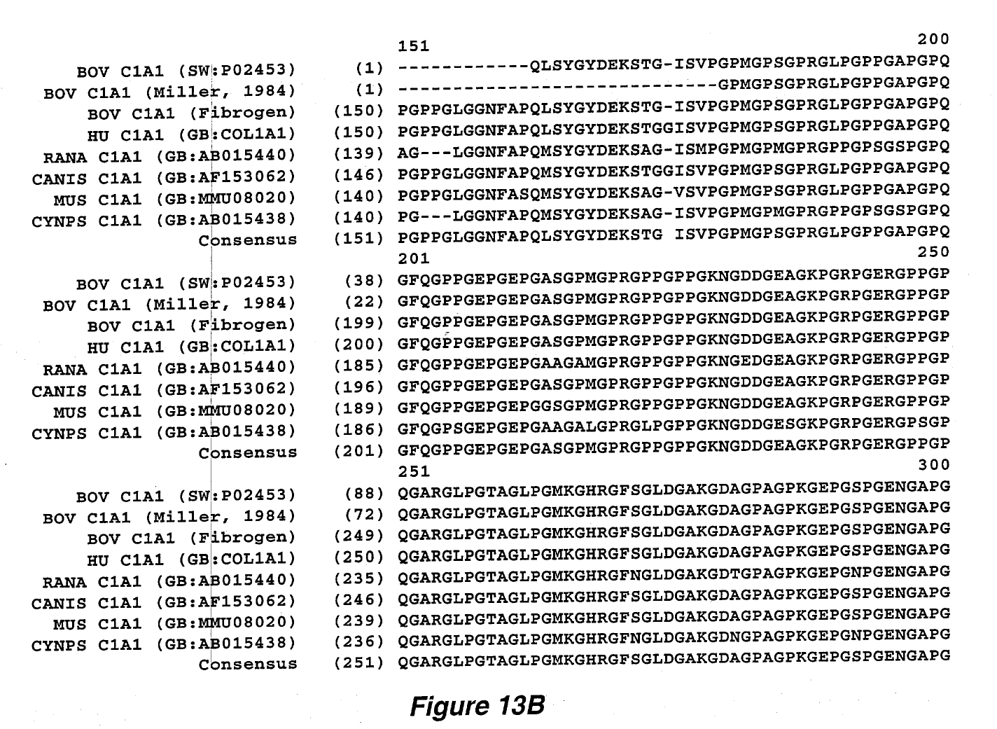

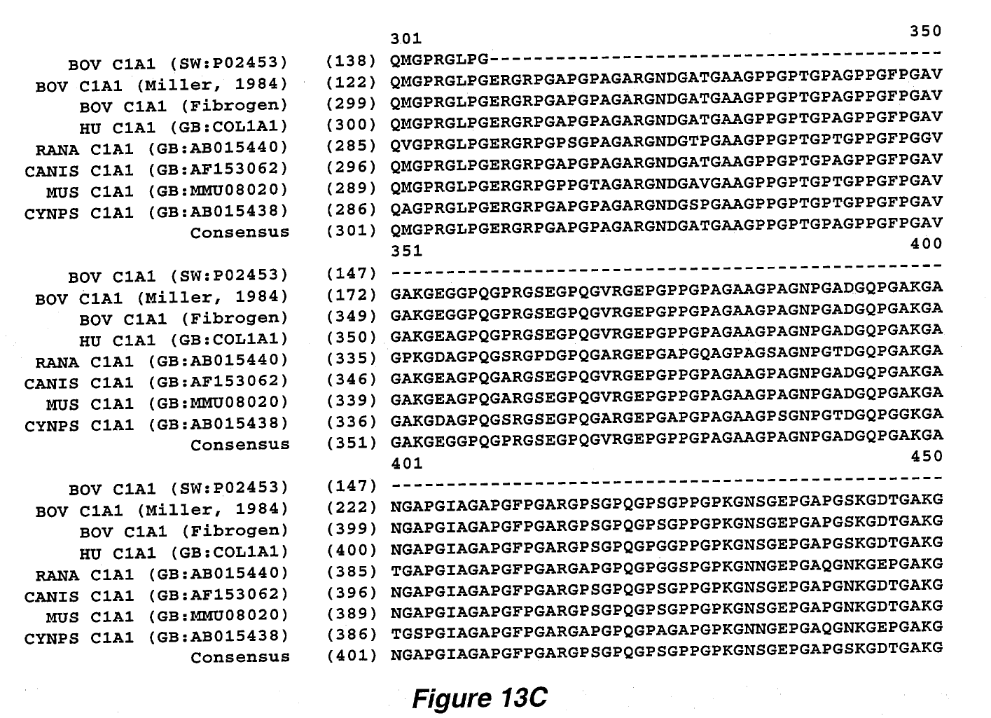

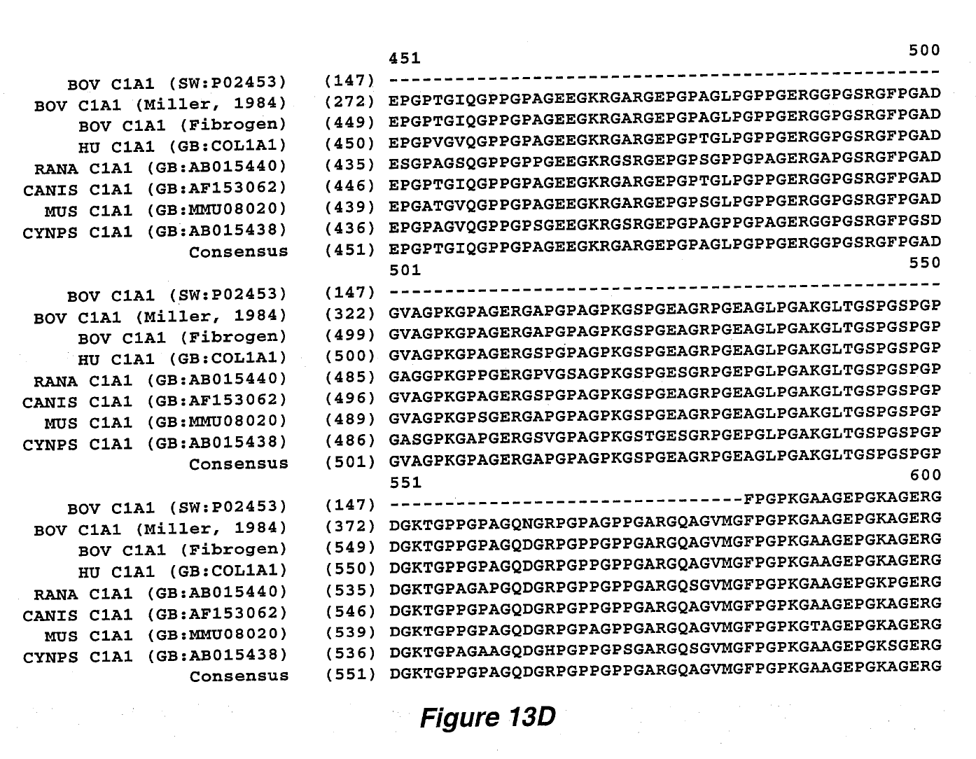

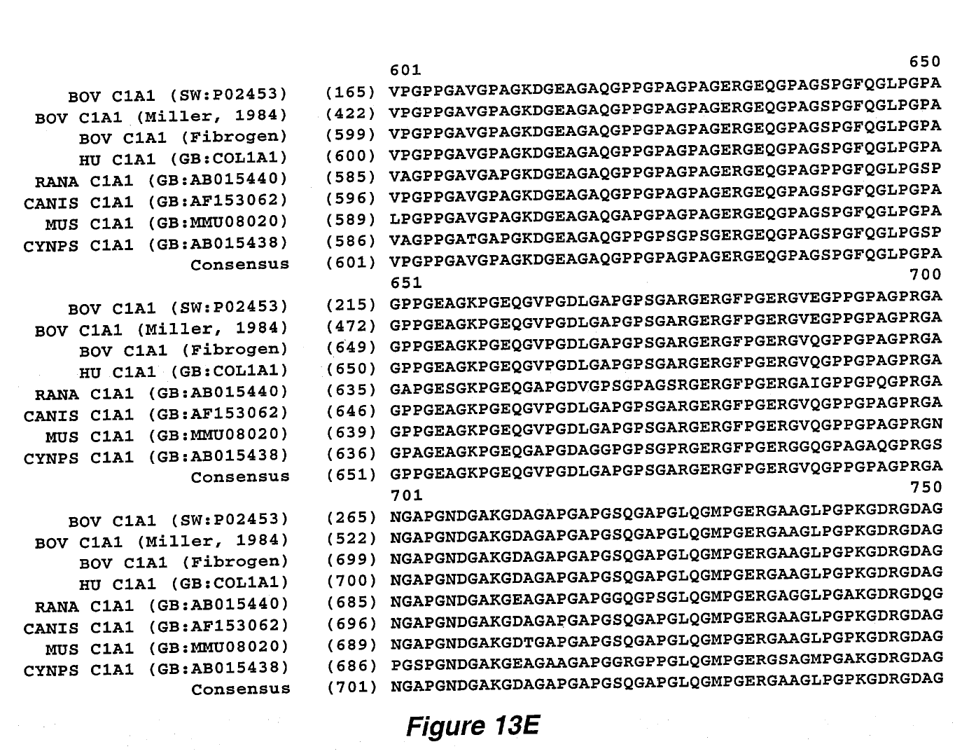

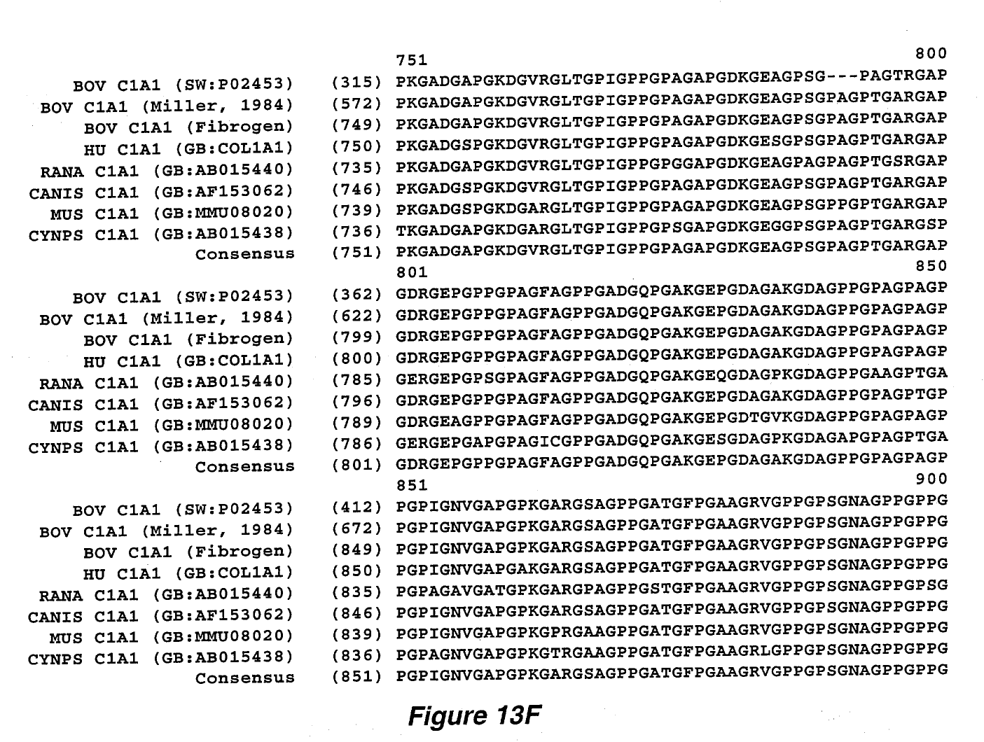

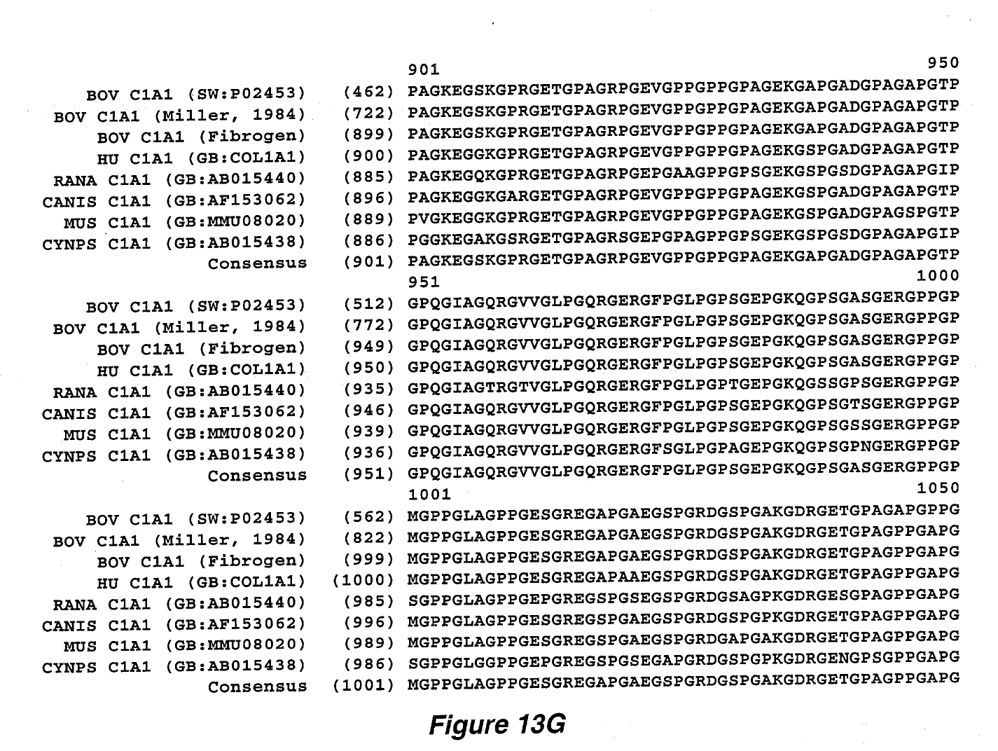

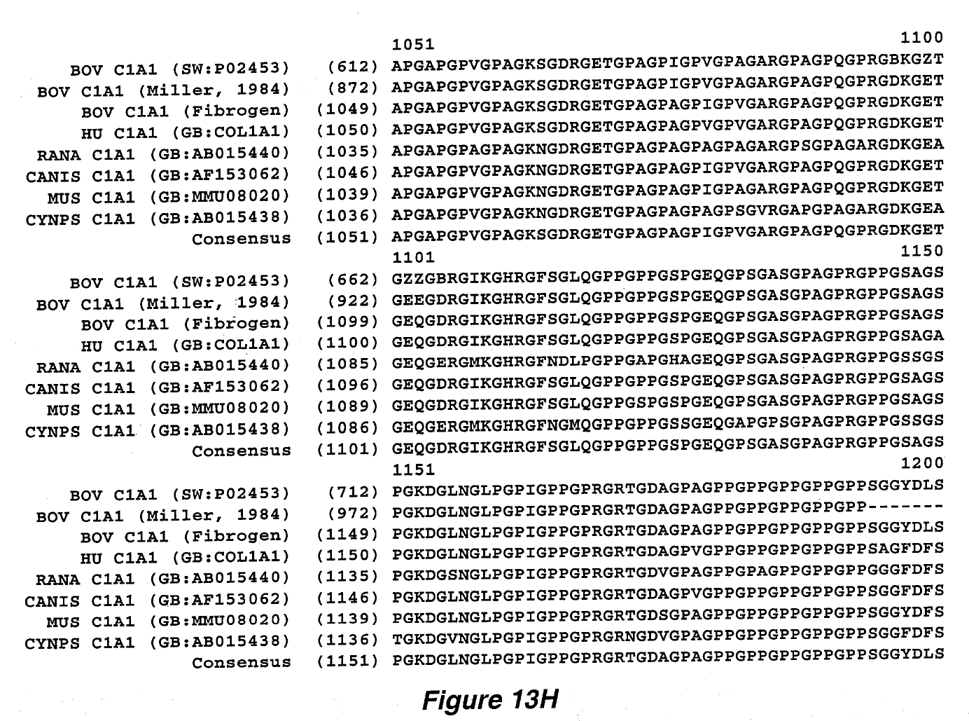

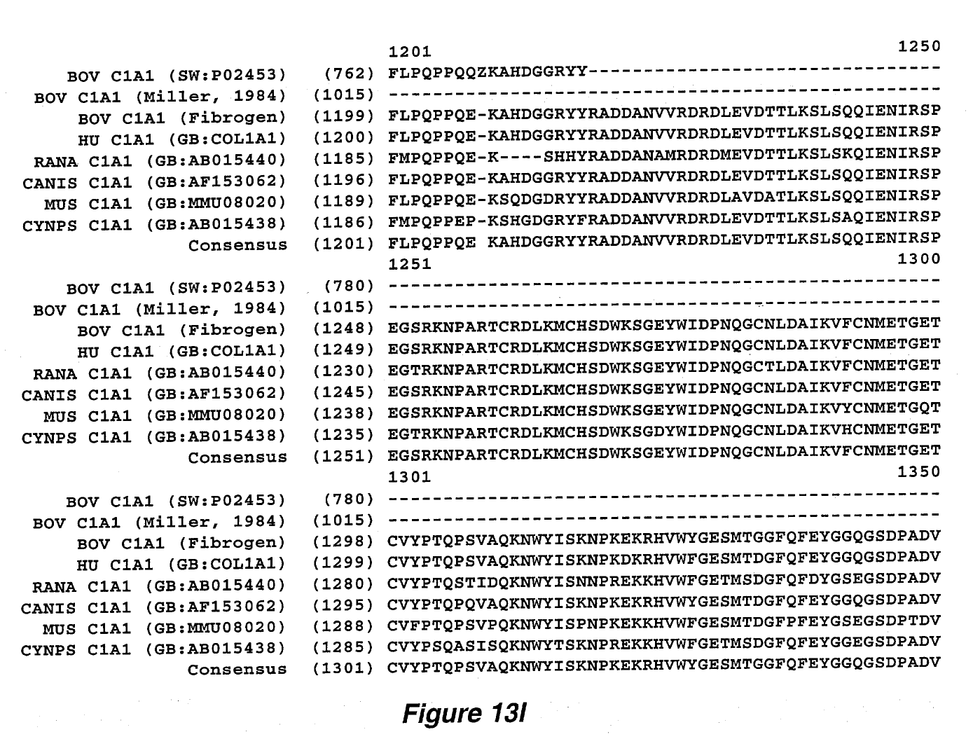

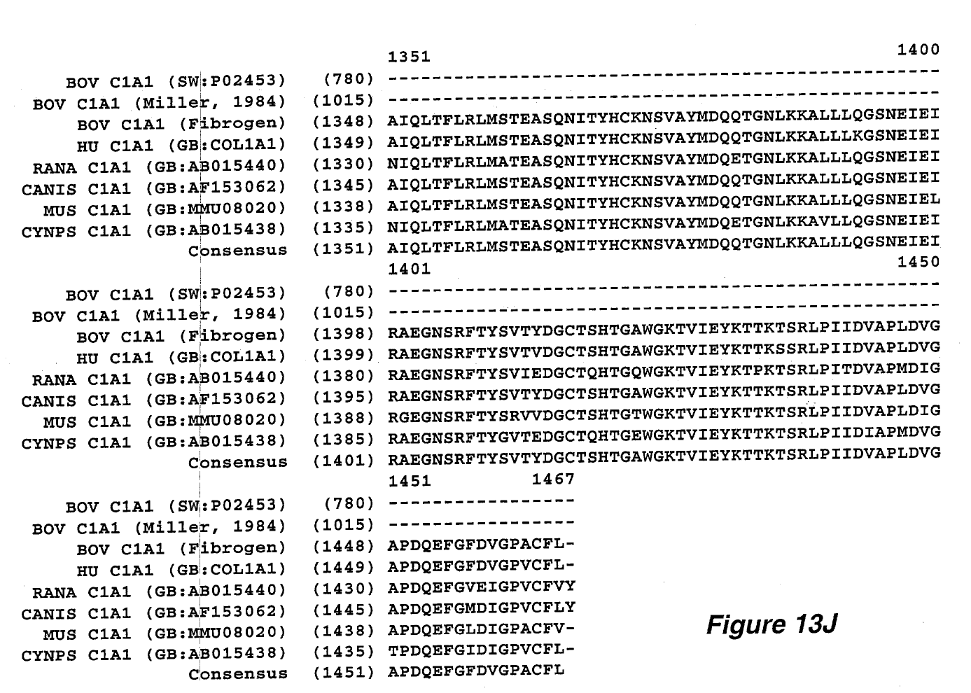

As shown in FIGS. 13A through 13I, translated bovine collagen ORF sequences were aligned with known human (HU), mouse (MUS), dog (CANIS), bullfrog (RANA), and Japanese newt (CYNPS) sequences. The translated bovine sequence also aligns with published amino acid sequence fragments of the triple helical repeat domains of bovine α1(I) collagen. (See, e.g., Miller (1984) Extracellular Matrix Biochemistry, ed. Piez, et al., Elsevier Science Publishing, New York, pp. 41-81; and SWISSPROT database accession number p02453.) Numerous differences between the predicted bovine α1(I) collagen protein sequence provided by the present invention and previously known bovine protein sequences were noted. Some of these differences include substitutions of amino acids that are typically difficult to distinguish by protein sequencing (i.e., glutamine/glutamic acid and aspartic acid/asparagine). The polynucleotide sequence disclosed herein as SEQ ID NO: 1 suggests these known bovine α1(I) collagen protein sequences may include errors, and therefore may, for example, be precluded for use in construction of a synthetic gene encoding authentic bovine α1(I) collagen gene by amino acid back-translation.

### Example 2

### Sequencing of Bovine Procollagen Type III α1

Bovine procollagen type III α1 cDNA was isolated as follows. Using 1 μl of Bovine Liver Poly A⁺ RNA (Clontech, Cat No. 6810-1), a cDNA strand was constructed with a reverse transcription reaction set up as follows using the Ambion Retroscript kit (Cat No. 1710):

| | |
|---|---|
| 1 μl | RNA (1 μg) |
| 4 μl | dNTPs mix (2.5 mM each) |
| 2 μl | Oligo dT first strand primers |
| 9 μl | Sterile water |

This solution was incubated at 75° C. for 3 min and then placed on ice. The following was then added:

| | |
|---|---|
| 2 μl | 10 X Alternative RT-PCR buffer |
| 1 μl | Placental RNAase inhibitor |
| 1 μl | M-MLV reverse transcriptase |

The reaction was allowed to proceed at 42° C. for 90 min and inactivated by incubation at 92° C. for 10 min. The reaction was then stored at −20° C.

Oligonucleotide primers were designed based on the sequence from the human procollagen type 3 α1 cDNA (Genbank Accession No. X14420) and the bovine procollagen type 3 α1 cDNA (Genbank Accession No. L47641). PCR was performed using the first strand cDNA prepared above and the primers as set forth in Table 5.

TABLE 5

| | | |
|---|---|---|
| SEQ ID NO: | PRIMER | SEQUENCE |

| | | |
|---|---|---|
| 54 | CIII-1 | GACATGATGAGCTTTGTGCAAAAGG |
| 55 | CIII-6 | TTTGGTTTATAAAAAGCAAACAGGGCC |
| 56 | A3-N | TCTCATGTCTGATATTTAGACATG |
| 57 | CIII-4 | GGACTAATGAGGCTTTCTATTTGTCC |
| 58 | CIII-2 | GGCACCATTCTTACCAGGCTCACC |
| 59 | CIII-3 | TGGGTCCCGCTGGCATTCCTGG |
| 60 | CIII-5 | CCAGGACAACCAGGCCCTCCTGG |

The PCR reaction conditions were as follows:

| | |
|---|---|
| 5 μl | Reverse transcriptase reaction above |
| 5 μl | 10 X Reaction Buffer |
| 1.5 μl | dNTPs mix (2.5 mM each) |
| 1.5 μl | Primer CIII-1 (5 μM) |
| 1.5 μl | Primer CIII-6 (5 μM) |
| 0.5 μl | Platinum pfx polymerase (Life Tech., Cat. No. 11708-013) |
| 35 μl | Sterile Water |
| 50 μl | Total Volume |

The reaction mixture was cycled in a Techne Genius DNA Thermal Cycler as follows:

| | | |
|---|---|---|
| 80° C. | 2 min | |
| 94° C. | 2 min | for 1 cycle |
| 94° C. | 30 sec | |
| 55° C. | 30 sec | for 35 cycles |
| 68° C. | 4.5 min | |
| 68° C. | 5 min | for 1 cycle |

A DNA band of approximately 4500 bp was identified in the reaction using primers CIII-1 (SEQ ID NO: 54) and CIII-6 (SEQ ID NO: 55). This DNA fragment was purified using a Qiagen QiaQuick Gel Extraction Kit (Cat No. 28704), and ligated to plasmid vector pCR ®-Blunt (Invitrogen Zero Blunt™ PCR Cloning Kit, Cat NO. K2700-20). The resultant recombinant plasmids were introduced into competent E. coli (JM109) and stocks of recombinant plasmid DNA generated using the Qiagen Qiaprep Spin Miniprep Kit (Cat No. 27106). DNA was sequenced on an LI-COR 4200 Automated Fluorescent Sequencer (MWG-Biotech UK Ltd.).

In areas where high quality sequence was available from partial bovine sequence as described in Genbank Accession Nos. L47641 and P04258 (amino acid only), the sequences of the bovine α1(III) cDNA of the present invention were shown to be identical. In other areas, sequence highly homologous to the human procollagen α1(III) cDNA (Genbank Accession No. X14420) and porcine procollagen α1(III) cDNA (Genbank Accession Nos. C94995, C94535, and C94565) was identified.

Since the 5′ primer CIII-1 (SEQ ID NO: 54) was designed using to the human sequence and was thus integrated into the newly isolated cDNA, the native bovine sequence was identified in this area as follows. An additional PCR fragment of approximately 3700 bp was amplified from bovine cDNA using primers A3-N (SEQ ID NO: 56) and CIII-4 (SEQ ID NO: 57). Primer A3-N was designed according to the sequence of the human procollagen type 3 α1 cDNA, in the region immediately upstream of the start codon. The resulting fragment was sequenced and confirmed using primers CIII-1 (SEQ ID NO: 54) and CIII-6 (SEQ ID NO: 55).

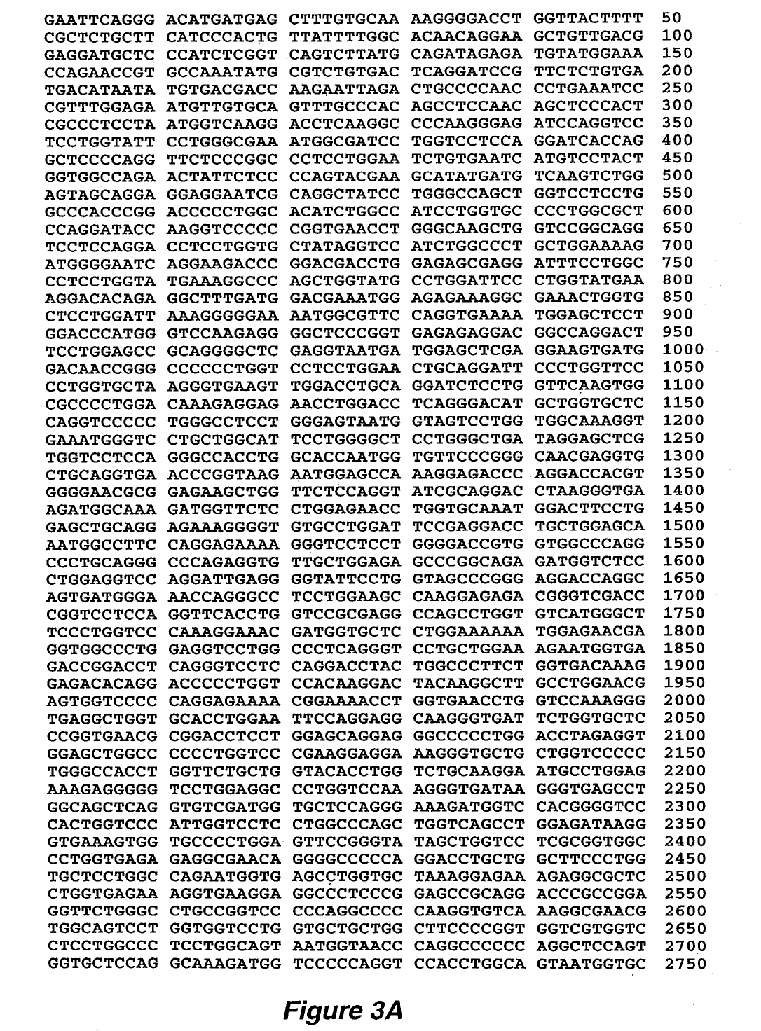

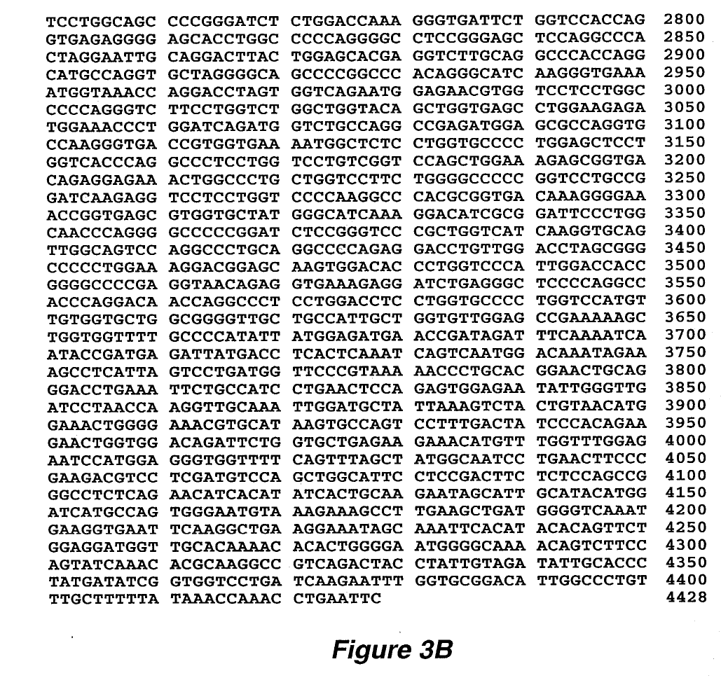

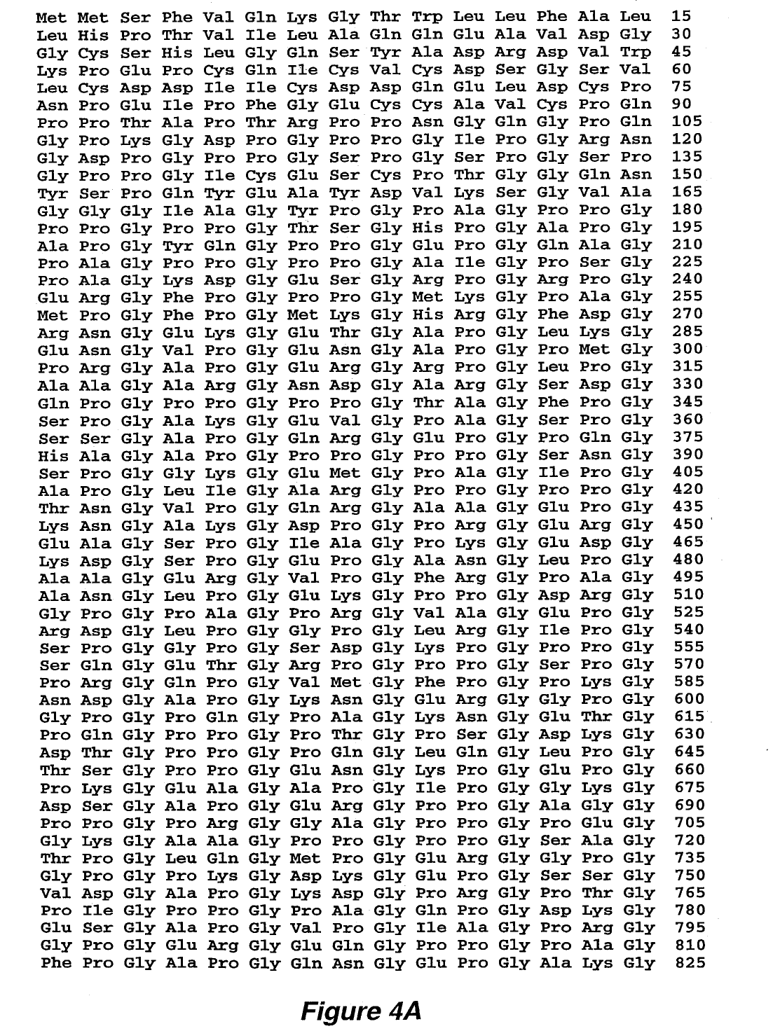

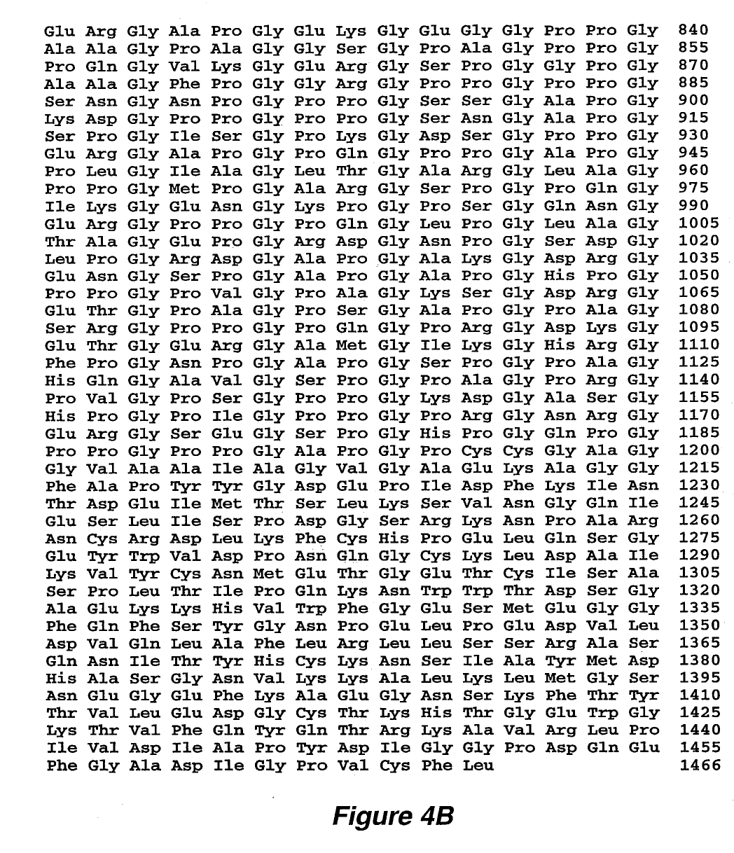

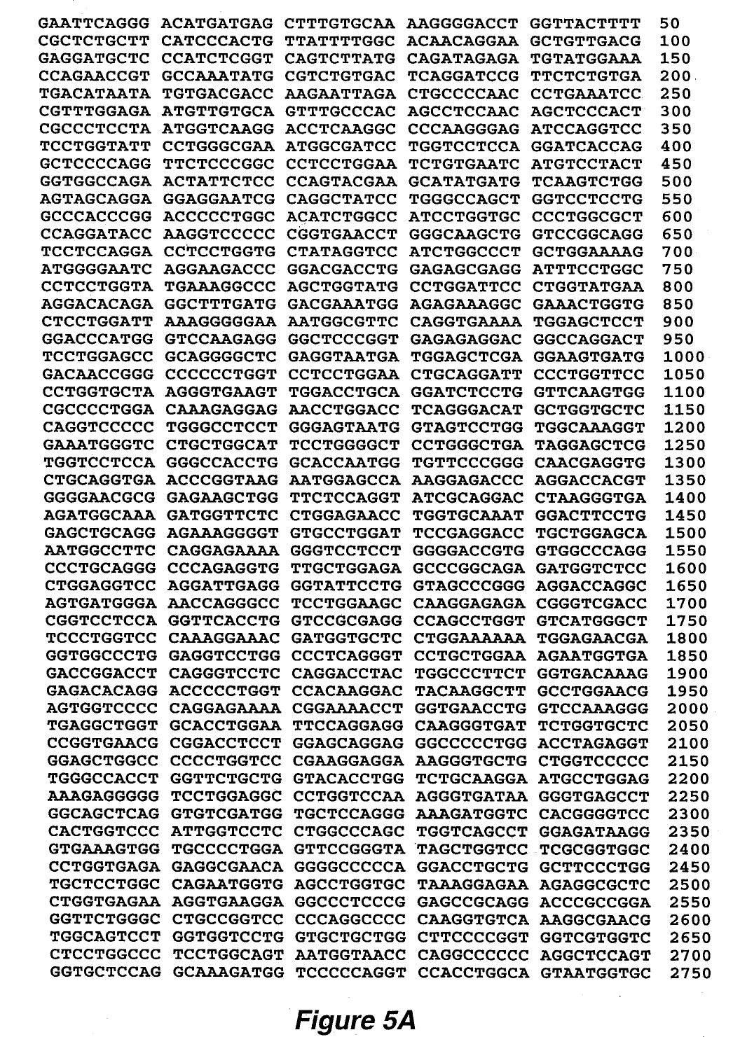

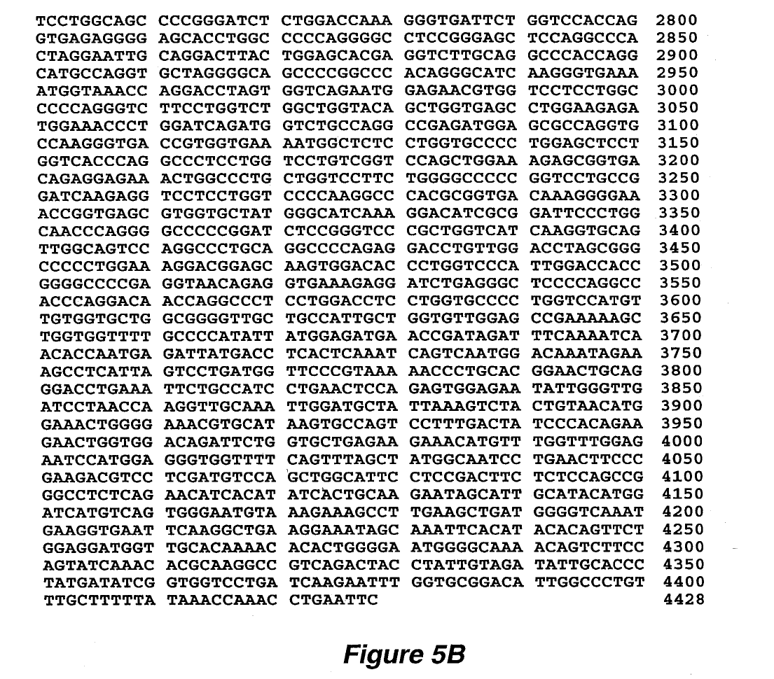

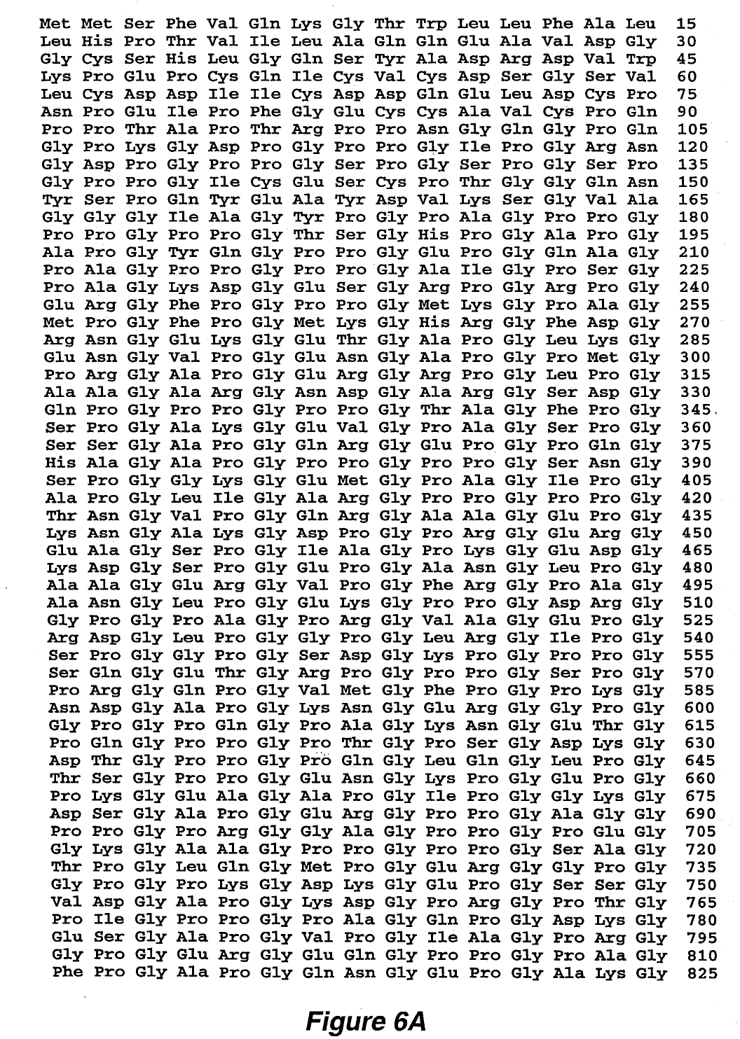

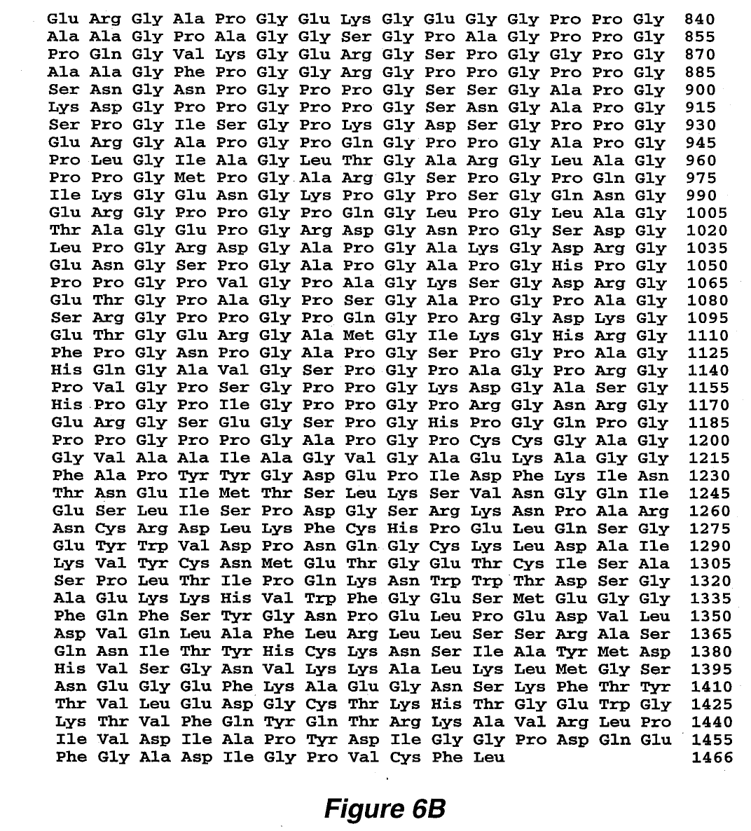

In summary, full length cDNA for bovine procollagen α1(III) was isolated by RT-PCR from bovine mRNA. Following extensive sequencing (three independent PCR reactions) using primers described in Table 5 and sequencing primers designed using methods described in Example 1 and methods known to those of skill in the art, 4428 bp of contiguous sequence containing the start codon ATG and stop codon TAA was assembled (FIGS. 3A through 3C, SEQ ID NO: 3). The deduced amino acid sequence is shown in FIGS. 4A through 4D (SEQ ID NO: 4). Two cDNA sequence variants of bovine α1(III) collagen (SEQ ID NO: 3 and SEQ ID NO: 5) were obtained and confirmed by sequencing of multiple clones. SEQ ID NO: 3 and the corresponding amino acid sequence (SEQ ID NO: 4) correspond to the appropriate region within the sequence of Genbank Accession No. L47641. Comparatively, SEQ ID NO: 5 (FIGS. 5A through 5C) displayed a C to T base substitution, leading to the codon change AAC to AAT (both encoding Asp); an A to G base substitution, leading to the codon change AAT to GAT (Asp to Asn substitution as residue 1232); and a T to C base subtitution, leading to the codon change GTC to GCC (Val to Ala substitution at residue 1382). The corresponding deduced amino acid sequence is shown in FIGS. 6A through 6D (SEQ ID NO: 6). The above sequences were identical to available partial bovine sequences (Genbank Accession Nos. L47641 and PO4258).

### Example 3

### Sequencing of Porcine Procollagen Type 1 α1

Porcine procollagen type I α1 cDNA was isolated using the following methods. Frozen porcine liver (obtained from Anglo Dutch Meats, Charing, Kent) was placed in liquid nitrogen and pulverized with a pestle and mortar. Approximately 800 mg of the crushed material was added to 5 ml lysis binding solution as described in the Ambion RNAqeous Kit (Cat No. 1912). Following Dounce homogenization, any debris was removed by centrifugation (12,000×g, 2 min) and an additional 5 ml lysis binding solution was added to the homogenate. Ten milliliters of 64% ethanol was added, mixed, and the lysate/ethanol mixture was applied to the RNAqeous filter (Ambion). Each filter was loaded with 2×700 μl lysate/ethanol mixture and centrifuged (12,000×g, 1 min). The filters were then washed once with 700 μL Wash Solution No. 1 (Ambion) and twice with 500 μl Wash Solution No. 2/3 (Ambion), and centrifuged after each wash step with a final centrifugation step after the final wash (12,000×g, 15 sec). The RNA was eluted from the filter by applying 2×60 μl preheated (95° C.) Elution solution (Ambion) to the center of the filter and centrifugation (12,000×g, room temp, 30 sec). The four eluates of four purifications of RNA (total concentration ˜15 μg) were pooled and precipitated with 0.5×vol lithium chloride (Ambion) overnight at −20° C. This was then centrifuged at 12,000×g, 15 min, 4° C., and the pellet washed with 70% ethanol. The pellet was then air dried and resuspended in 15 μl sterile water and stored at −70° C.

Using 1 μl of the RNA isolated above, a cDNA strand was constructed, using the reverse transcription reaction performed as described above in Example 2. Oligonucleotide primers based on the sequence from the human procollagen α1(I) cDNA (Genbank Accession No. NM000088) and the porcine procollagen α1(I) cDNA (Genbank Accession No. C94935) were designed. PCR was then performed, using methods described in Example 2, with the first strand cDNA prepared and primers corresponding to known human or porcine DNA (Table 6).

TABLE 6

| | | |
|---|---|---|
| SEQ ID NO | PRIMER | SEQUENCE |

| | | |
|---|---|---|
| 61 | HU1-5 | GACATGTTCAGCTTTGTGGACCTC |
| 62 | PCA1-6 | AGTTTACAGGAAGCAGACAG |
| 63 | A1-N | CTACATGTCTAGGGTCTAGACATG |
| 64 | PCA1-4 | AGGCGCCAGGCTCGCCAGGCTCAC |
| 65 | PCA1-3 | AGTTGTCTTATGGCTATGATGAG |

The reverse transcriptase-PCR was carried out on RNA purified from porcine liver and a DNA band of approximately 4500 bp was identified in the reaction, using primers HU1-5 (SEQ ID NO: 61) and PCA1-6 (SEQ ID NO: 62). This DNA fragment was purified, cloned, and sequenced as described in Example 2.

Since the 5′ primer HU1-5 (SEQ ID NO: 61) was designed according to the human sequence and thus was integrated into the newly isolated cDNA described above, the native porcine sequence needed to be confirmed in this area. An additional PCR fragment of approximately 750 bp was consequently amplified from porcine cDNA using primers Al-N (SEQ ID NO: 63) and PCAI-4 (SEQ ID NO: 64). Primer A1-N (SEQ ID NO: 63) was designed according to the sequence of the human procollagen αI(I) cDNA in the region immediately upstream of the start codon. This fragment was sequenced to confirm that the full-length porcine α1(I) cDNA fragment generated using primers HU1-5 (SEQ ID NO: 61) and PCA1-6 (SEQ ID NO: 62) had the authentic porcine 5′ end rather than a hybrid sequence introduced by the human sequence based primer.

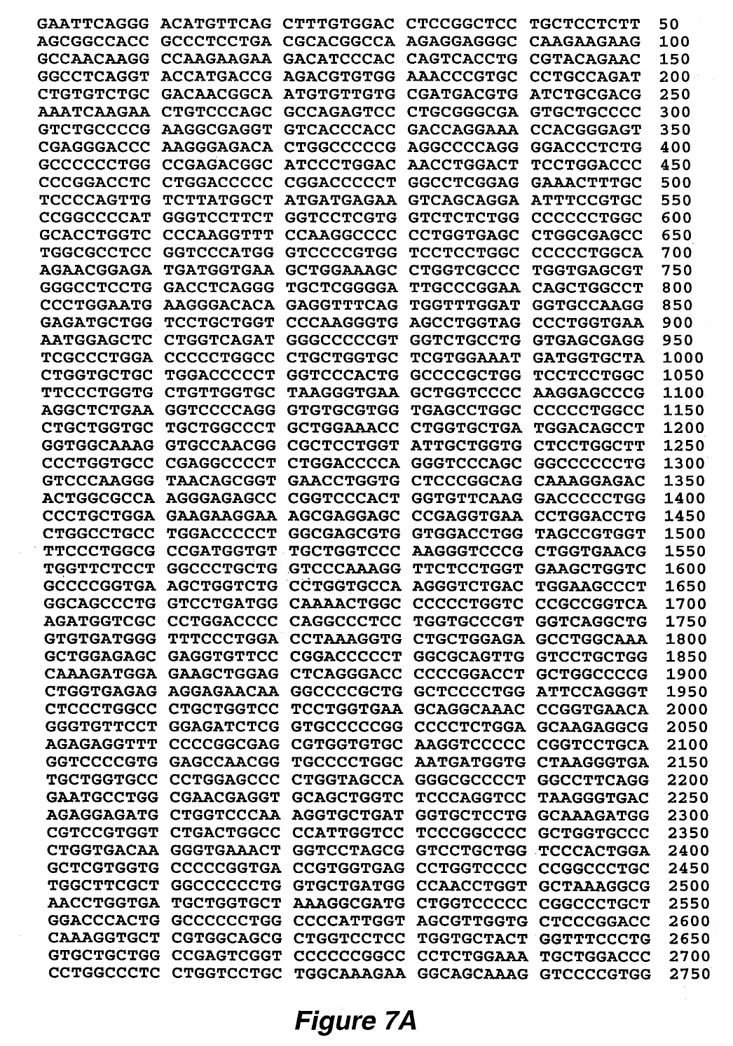

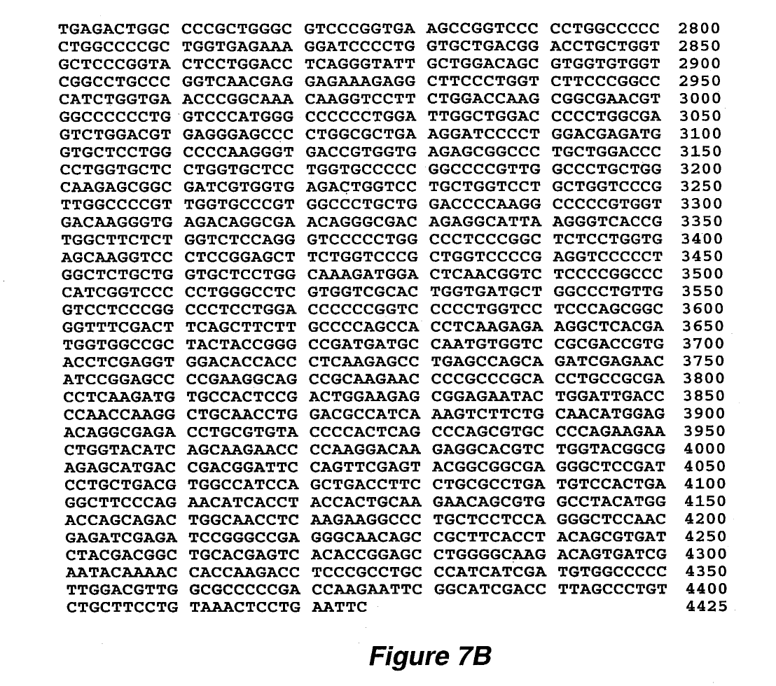

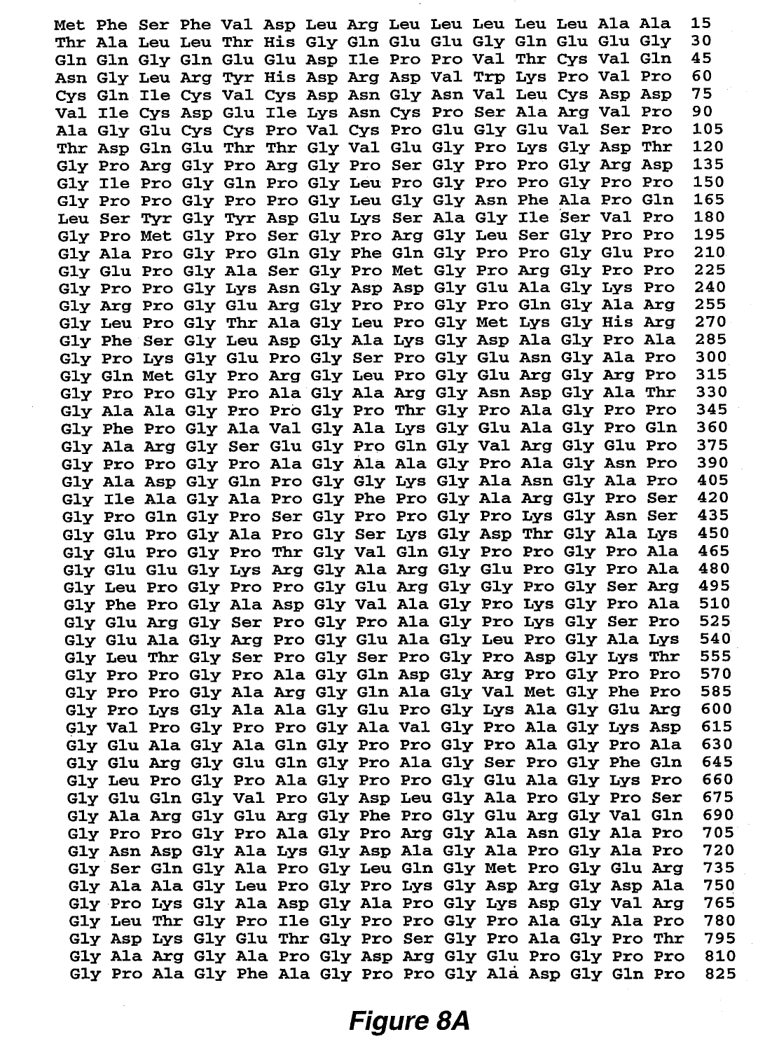

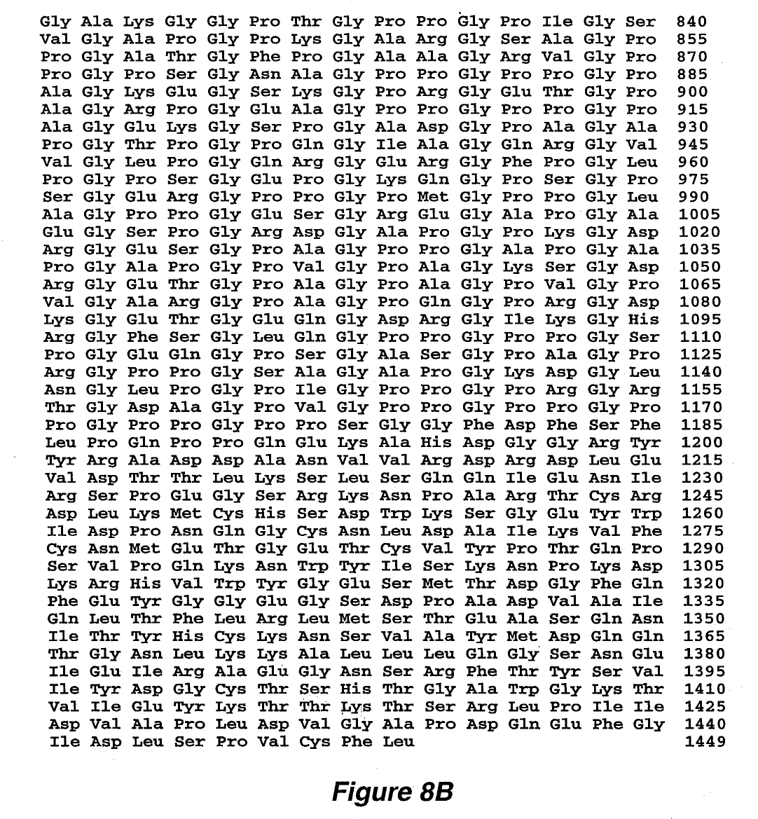

In summary, full-length cDNA for porcine procollagen α1(I) was isolated by RT-PCR from porcine liver. Following extensive sequencing (three independent PCR reactions), 4425 bp of contiguous sequence containing the start codon ATG and stop codon TAA was assembled as shown in FIGS. 7A through 7C (SEQ ID NO: 7). This sequence was identical to the available partial porcine sequence (Genbank Accession Nos. C94935 and AU058670). The sequence shows a high degree of homology to the human procollagen type 1 α1 sequence (Accession No. G4502944). The corresponding amino acid sequence of the porcine type 1 α1 collagen is shown in FIGS. 8A through 8D (SEQ ID NO: 8).

### Example 4

### Sequencing of Porcine Procollagen Type I α2

Porcine procollagen type I α2 cDNA was isolated using the following methods. Total RNA isolation, reverse transcription, and PCR were performed essentially as described above in Example 2. Oligonucleotide primers were designed based on the sequence from the human α2(I) procollagen (Genbank Accession No. NM000089) and the porcine α2(I) procollagen (Genbank Accession No. AU058497). Primers used are set forth in Table 7.

TABLE 7

| | | |
|---|---|---|
| SEQ ID NO | PRIMER | SEQUENCE |

| | | |
|---|---|---|
| 66 | HU2-5 | GACATGCTCAGCTTTGTGGATACG |
| 67 | PCA2-6 | AGCTGGACCAGGCTCACCAACAA |
| 68 | PCA2-5 | TGGTGCTAAGGGTGCTGCTGGCCT |
| 69 | PCA2-8 | AGGTTCACCCACTGATCCAGCAACA |
| 70 | PCA2-7 | TCCCTCTGGAGAGCCTGGTACTGCT |
| 71 | PCA2-2 | TGGAAGTTTGGGTTTTAAACTTCCC |
| 72 | A2-N | ACACAAGGAGTCTGCATGTCT |

The following primer pairs were used to generate three overlapping fragments of the following sizes: 1054 bp DNA, using primer HU2-5 (SEQ ID NO: 66) and primer PCA2-6 (SEQ ID NO: 67); 1766 bp DNA, using primer PCA2-5 (SEQ ID NO: 68) and primer PCA2-8 (SEQ ID NO: 69); and 1937 bp DNA, using primer PCA2-7 (SEQ ID NO: 70) and primer PCA2-2 (SEQ ID NO: 71). These DNA fragments were isolated, subcloned and sequenced using methods described above. Sequence highly homologous to the full-length human collagen α2(I) gene (Genbank Accession No. NM000089) or to the partial porcine α2(I) sequence (Genbank Accession No, AU058497) was identified.

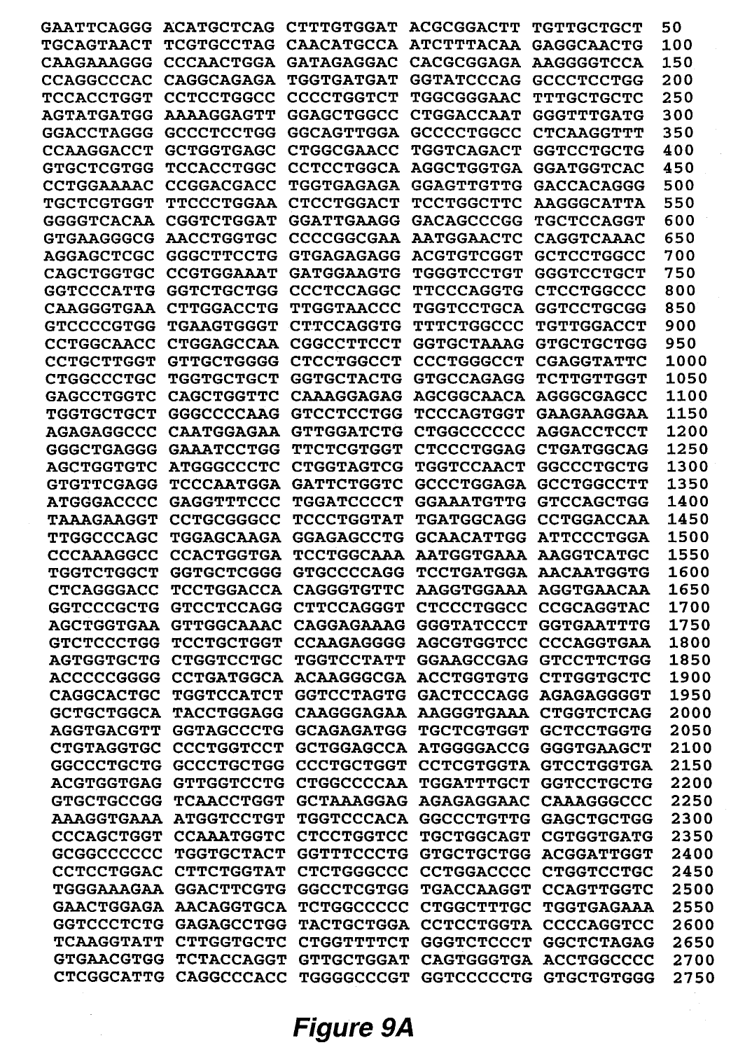

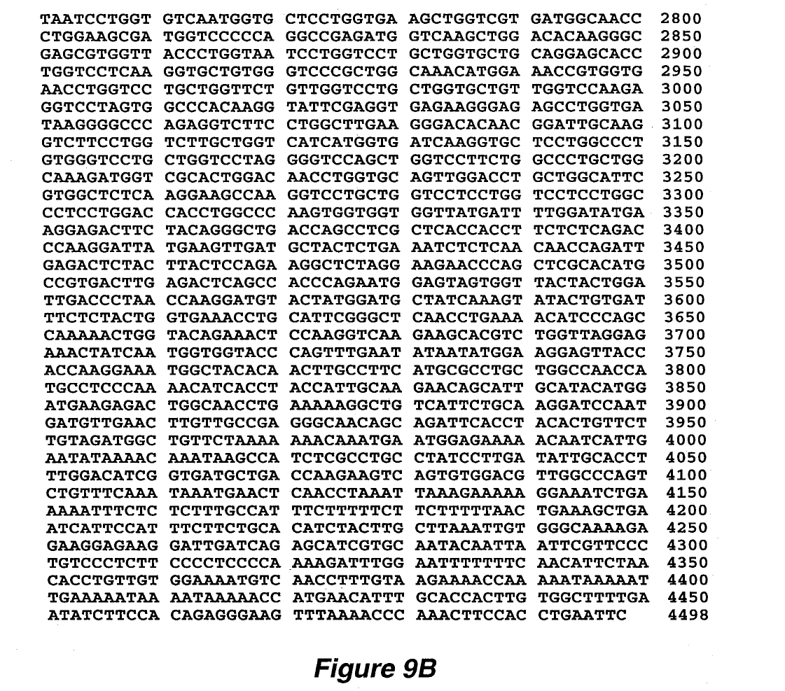

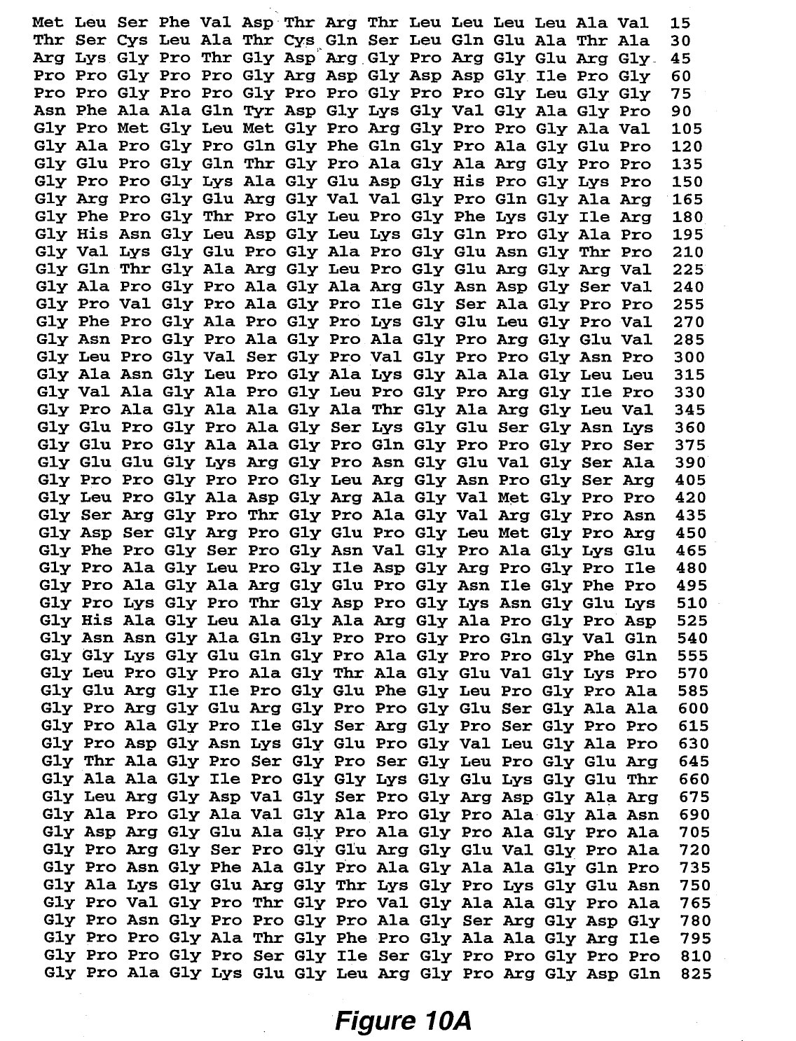

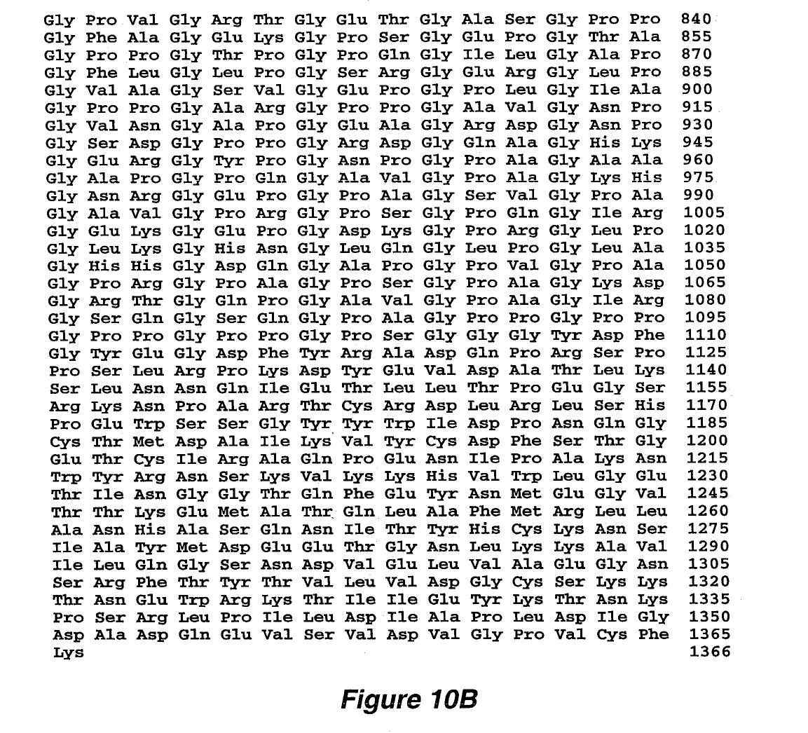

As the 5′ primer HU2-5 (SEQ ID NO: 66) used in the cloning of the porcine procollagen type 1 α2 cDNA was designed using to the human sequence and was thus integrated into the newly isolated cDNA, a further PCR fragment of approximately 1100 bp was consequently amplified from porcine cDNA using primers A2-N (SEQ ID NO: 72) and PCA2-6 (SEQ ID NO: 67). Primer A2-N had been designed according to the sequence of the human (Genbank Accession No. NM0000890) and bovine (Genbank Accession No. AB008683) procollagen α2(I) cDNA in the region immediately upstream of the start codon. The sequence of this DNA fragment confirmed that the full-length fragment generated using primers HU2-5 and PCA2-2 had the authentic porcine 5′ end. The full-length nucleotide sequence for the porcine α2(I) collagen gene is shown in FIGS. 9A through 9C (SEQ ID NO: 9). The corresponding amino acid sequence is described in FIGS. 10A through 10C (SEQ ID NO: 10).

### Example 5

### Sequencing of Porcine Procollagen Type III α1

Porcine procollagen type α1 cDNA was isolated using the following methods. Total RNA was isolated from frozen porcine liver, reverse transcription, and PCR was performed as described above in Example 2. Oligonucleotide primers were designed based on the sequence from the human procollagen type 3 α1 cDNA (Genbank Accession No. X14420) and the porcine procollagen type 3 α1 cDNA (Genbank Accession Nos. C94995, C94535, and C94565). These primers are set forth in Table 5 above.

RT-PCR was carried out on RNA purified from porcine liver and a DNA band of approximately 4500 bp was identified in the reaction using primers CIII-1 (SEQ ID NO: 54) and CIII-6 (SEQ ID NO: 55). This DNA fragment was purified, subcloned, and sequenced as described above. In areas where high quality sequence was available from partial porcine sequence as described in Genbank Accession Nos. C94565, C94535, and C95995, the sequence of the new cDNA was shown to be identical. In other areas sequence highly homologous to the human procollagen α1(III) cDNA (Genbank Accession No. X14420) and bovine procollagen α1(III) cDNA (sequences derived from the current inventions and Genbank Accession No. L47641) were identified.

As the 5′ primer CIII-1 was designed using the human sequence and was integrated into the newly isolated cDNA, the native porcine sequence needed to be confirmed. A further PCR fragment of approximately 3700 bp was consequently amplified from porcine cDNA using primers A3-N (SEQ ID NO: 56) and CIII-4 (SEQ ID NO: 57). Primer A3-N was designed according to the sequence of the human procollagen α1(III) cDNA in the region immediately upstream of the start codon. This fragment was sequenced to confirm that the full-length fragment generated using primers CIII-1 and CIII-6 had the authentic porcine 5′ sequence.

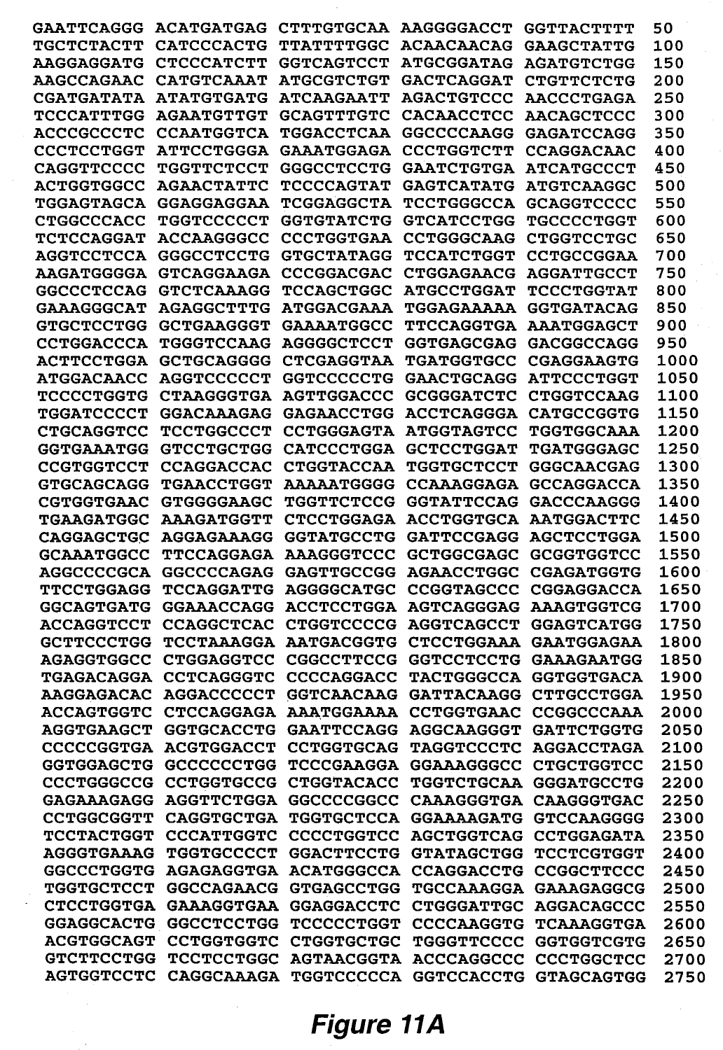

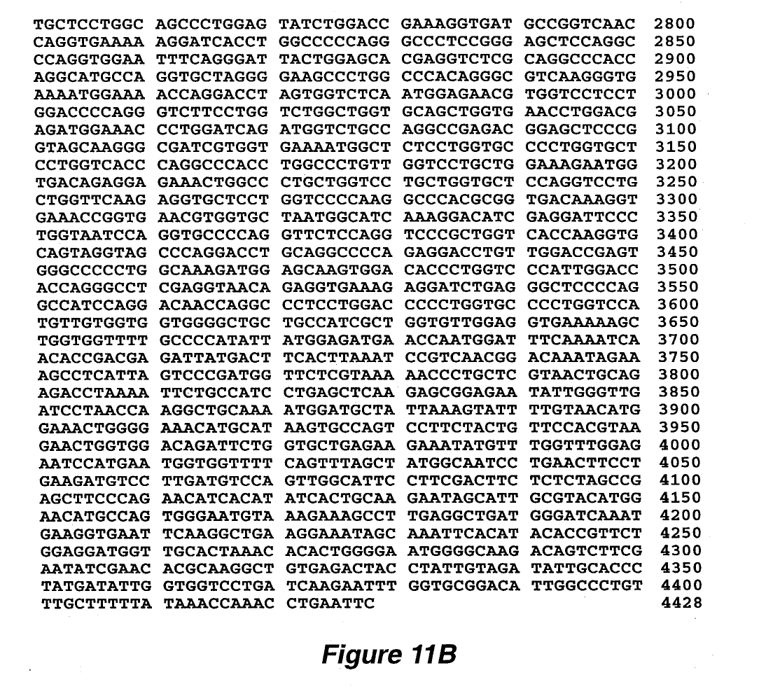

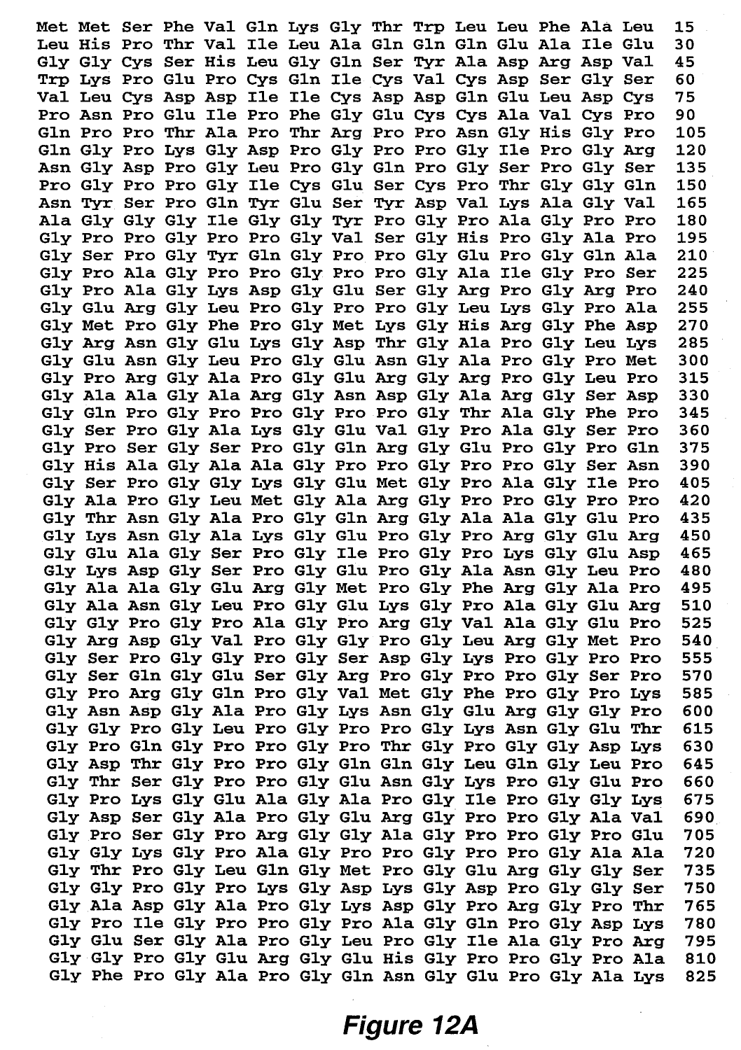

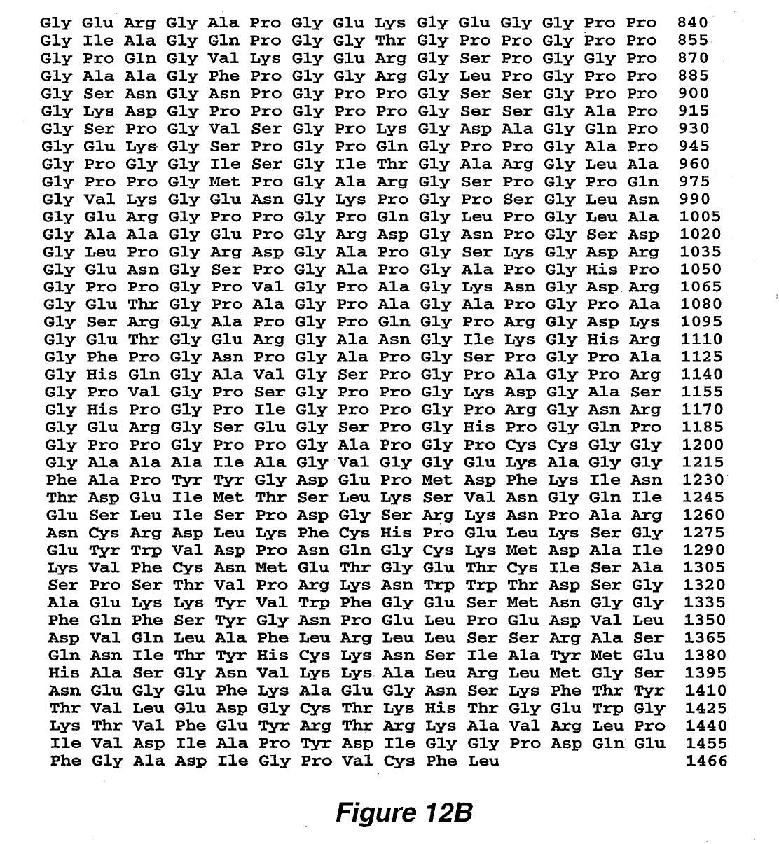

In summary, a full-length cDNA for porcine α1(III) procollagen was isolated by RT-PCR from porcine liver. Following extensive sequencing (three independent PCR reactions) 4428 bp of contiguous sequence containing the start codon ATG and stop codon TAA was assembled. (FIGS. 11A through 11C, SEQ ID NO: 11.). This sequence was identical to available partial porcine sequence (Genbank Accession Nos. C94565, C94535, and C95995). Overall the sequence showed a high degree of homology to the human α1(III) procollagen cDNA (Genbank Accession No. X14420) and bovine of α1(III) procollagen cDNA (from the current invention and Genbank Accession Nos. L47641 and P04258). The deduced amino acid sequence for porcine type III α1 collagen is presented in FIGS. 12A through 12C (SEQ ID NO: 12).

### Example 6

### Production of Animal Collagens and Gelatins in Transgenic Plants

The cDNAs encoding an animal collagen of the present invention, an α subunit of prolyl 4-hydroxylase, and a β subunit of prolyl 4-hydroxylase are cloned into an appropriate plant expression vector that contains the necessary elements to properly express a foreign protein. Such elements may include, for example a signal peptide, promoter and a terminator. (See, e.g., Rogers et al., supra; Schardl et al., supra, Berger et al., supra.) For example, pVL vectors have been described in the art. (See, e.g., A. Lamberg et al. (1996) J. Biol. Chem.271:11988-11995.) These recombinant pVL vectors are used as a gene source for the construction of plant expression vectors using conventional methods known in the art. In order to express the collagen in plant or plant cells, the nucleic acid sequences are operably linked, for example, to a CaMV 35S promoter. The nucleic acid sequences encoding an α subunit or βsubunit of prolyl 4-hydroxylase are operably linked to a CaMV 35S promoter, and may be present on the same plasmid or on different plasmids to produce a biologically active prolyl 4-hydroxylase.

The expression vectors are transformed into plants or plant cells using transformation techniques well known in the art. The expression clones are selected by, for example, northern and western blotting, and can be cultivated in a fermentor to generate a cell mass for purification of recombinant collagen.

The expression of the α subunit and the β subunit of prolyl 4-hydroxylase and animal collagen is screened, for example, by immunoblotting using three hundred (300) mg cell pellets extraction in 10 mM Tris, pH 7.8, 100 mM NaCl, 100 mM Glycine, 10 uM DTT, 0.1% Triton X100, 2 uM Leupeptin, and 0.25 mM PMSF. The proteins in the extract are separated with 4-20% SDS-PAGE, and transferred to a nitrocellulose membrane to be probed with antibodies against the α subunit and β subunit of prolyl 4-hydroxylase and the animal collagen.

To characterize recombinant animal collagen produced in plants or plant cells, the following protocol is carried out:

1. Suspend and homogenize cell pellets in 1M NaCl, 0.05M Tris, pH7.4 and stir for 1 hour at

4° C. Collect the supernatant by centrifugation at 4° C.;

2. Add 7.5ml acetic acid to the supernatant and incubate at 4° C. for 2 hours. Collect the pellet by centrifugation at 4° C.;

3. Wash the pellet twice with 2M NaCl, 0.05M Tris, pH 7.4;

4. Re-dissolve in 2M Urea, 0.2M NaCl, 0.05M Tris, pH 7.4;

5. Dialyze against 2M Urea, 0.2M NaCl, 0.05M Tris, pH 7.4;

6. Run through a DEAE-cellulose column. Collect the flow-through;

7. Add acetic acid to 0.5M and add NaCl to 0.9M and incubate for 2 hours at 4° C.;

8. Collect pellets by centrifugation;

9. Resuspend the pellet in 0.5M acetic acid and stir overnight at 4° C.;

10. Digest the pellet with 0.1 mg/ml pepsin for 2 hours;

11. Add saturated Tris buffer and adjust pH to 7.4;

12. Incubate overnight to inactivate pepsin;

13. Add NaCl to 0.9M and acetic acid to 0.5M, Incubate for 2 hours at 4° C.;

14. Collect the pellet by centrifugation at 4° C.;

15. Wash the pellet with 2M NaCl, 0.05M Tris, pH 7.4;

16. Dissolve in 2M Urea, 150M NaCl and 0.05M Tris, pH 7.4; and

17. Heat the sample at 56° C. for 5 min and then load to Bio-Gel TSK 40 column operated by HPLC system.

The resulting purified collagen is characterized by amino acid composition analysis.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims. All references cited herein are incorporated by reference herein in their entirety.

What is claimed is:

1. A composition comprising a recombinant bovine collagen.

2. The composition of claim 1, wherein the recombinant bovine collagen is selected from the group consisting of recombinant bovine type I collagen and recombinant bovine type III collagen.

3. The composition of claim 1, wherein the recombinant bovine collagen is selected from the group consisting of:
(a) recombinant bovine α1(I) collagen;
(b) recombinant bovine α2(I) collagen;
(c) recombinant bovine α1(III) collagen; and
(d) fragments or variants thereof.

4. The composition of claim 1, wherein the recombinant bovine collagen comprises at least one polypeptide selected from the group consisting of:
(a) SEQ ID NO: 2;
(b) SEQ ID NO: 4;
(c) SEQ ID NO: 6; and
(d) fragments or variants thereof.

5. The composition of claim 1, wherein the recombinant bovine collagen is encoded by a polynucleotide selected from the group consisting of:
(a) SEQ ID NO: 1;
(b) SEQ ID NO: 3;
(c) SEQ ID NO: 5; and
(d) fragments or variants thereof.

6. A recombinant bovine collagen of one type of collagen free of any other type of collagen.

7. A composition comprising a recombinant bovine gelatin.

8. The composition of claim 7, wherein the recombinant bovine gelatin is obtained from recombinant bovine collagen.

9. The composition of claim 8, wherein the recombinant bovine collagen is selected from the group consisting of recombinant bovine type I collagen and recombinant bovine type III collagen.

10. The composition of claim 7, wherein the recombinant bovine gelatin is produced directly from an altered collagen construct.

11. The composition of claim 7, wherein the recombinant bovine gelatin is obtained from one type of recombinant bovine collagen free of any other type of collagen.

12. The composition of claim 7, wherein the recombinant bovine gelatin is obtained from a recombinant bovine collagen comprising a polypeptide selected from the group consisting of:
(a) recombinant bovine α1(I) collagen;
(b) recombinant bovine α2(I) collagen;
(c) recombinant bovine α1(III) collagen; and
(d) fragments or variants thereof.

13. The composition of claim 7, wherein the recombinant bovine gelatin is obtained from a recombinant bovine collagen comprising a polypeptide selected from the group consisting of:
(a) SEQ ID NO: 2;
(b) SEQ ID NO: 4;
(c) SEQ ID NO: 6; and
(d) fragments or variants thereof.

14. The composition of claim 7, wherein the recombinant bovine gelatin is obtained from a recombinant bovine collagen comprising a polypeptide encoded by a polynucleotide selected from the group consisting of:
(a) SEQ ID NO: 1;
(b) SEQ ID NO: 3;
(c) SEQ ID NO: 5; and
(d) fragments or variants thereof.

15. An isolated and purified polypeptide comprising a sequence selected from the group consisting of:
(a) SEQ ID NO: 2;
(b) SEQ ID NO: 4;
(c) SEQ ID NO: 6; and
(d) fragments or variants thereof.

16. An isolated and purified polynucleotide comprising a sequence selected from the group consisting of:
(a) SEQ ID NO: 1;
(b) SEQ ID NO: 3;
(c) SEQ ID NO: 5; and
(d) fragments and variants thereof.

17. A recombinant host cell comprising the polynucleotide of claim 16.

18. A transgenic animal comprising the polynucleotide of claim 16.

19. A transgenic plant comprising the polynucleotide of claim 16.

20. A pharmaceutical composition comprising a recombinant bovine collagen.

21. A pharmaceutical composition comprising a recombinant bovine gelatin.

22. A method for producing a recombinant bovine collagen, the method comprising:
(a) introducing into a host cell at least one polynucleotide encoding a bovine collagen;
(b) culturing the host cell under conditions suitable for expression; and
(c) recovering the recombinant bovine collagen.

23. The method of claim 22, wherein the at least one polynucleotide comprises a sequence encoding a bovine collagen selected from the group consisting of:
(a) bovine type I collagen;
(b) bovine type III collagen;
(c) bovine type I procollagen;
(d) bovine type III procollagen; and
(e) fragments and variants thereof.

24. The method of claim 22, wherein the at least one polynucleotide comprises a sequence encoding a bovine collagen selected from the group consisting of:
(a) bovine α1(I) collagen;
(b) bovine α2(I) collagen;
(c) bovine α1(III) collagen; and
(d) fragments or variants thereof.

25. The method of claim 22, wherein the at least one polynucleotide comprises a sequence encoding a bovine collagen selected from the group consisting of:
(a) SEQ ID NO: 2;
(b) SEQ ID NO: 4;
(c) SEQ ID NO: 6; and
(d) fragments or variants thereof.

26. The method of claim 22, wherein the at least one polynucleotide comprises a sequence selected from the group consisting of:
(a) SEQ ID NO: 1;
(b) SEQ ID NO: 3;
(c) SEQ ID NO: 5; and
(d) fragments and variants thereof

27. The method of claim 22, wherein the host cell is selected from the group consisting of a prokaryotic cell, a eukaryotic cell, an animal cell, a yeast cell, a plant cell, an insect cell, and a fungal cell.

28. A method for producing a recombinant bovine collagen, the method comprising:
(a) introducing into a host cell at least one polynucleotide encoding a bovine collagen, and at least one polynucleotide encoding a post-translational enzyme important to the biosynthesis of collagen;
(b) culturing the host cell under conditions suitable for expression; and
(c) isolating the recombinant bovine collagen.

29. The method of claim 28, wherein the post-translational enzyme is selected from the group consisting of prolyl hydroxylase, lysyl hydroxylase, and lysyl oxidase.

30. A method for producing a recombinant bovine gelatin, the method comprising:
(a) providing recombinant bovine collagen; and
(b) obtaining the recombinant bovine gelatin therefrom.

31. A method for producing a recombinant bovine gelatin, the method comprising:
(a) producing recombinant bovine gelatin directly from an altered bovine collagen construct; and
(b) isolating the recombinant bovine gelatin.

32. A hard gel capsule comprising a recombinant bovine gelatin.

33. A soft gel capsule comprising a recombinant bovine gelatin.

34. An edible composition comprising a recombinant bovine gelatin.

35. A protein supplement comprising a recombinant bovine gelatin.

36. A nutraceutical comprising a recombinant bovine gelatin.

37. An injectable composition comprising a recombinant bovine gelatin.
